(19) United States
(12) Patent Application Publication
(10) Pub. No.: US 2006/0222636 A1
(43) Pub. Date: Oct. 5, 2006

(54) Compositions, methods and kits relating to reprogramming adult differentiated cells and production of embryonic stem cell-like cells

(75) Inventor: Anura Rambukkana, New York, NY (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: The Rockefeller University

(21) Appl. No.: 11/372,612

(22) Filed: Mar. 10, 2006

Related U.S. Application Data
(63) Continuation of application No. PCT/US04/29355, filed on Sep. 9, 2004.
(60) Provisional application No. 60/504,144, filed on Sep. 19, 2003.

(51) Int. Cl. A61K 48/00; C12N 5/08; C12N 15/00

(52) U.S. Cl. 424/93.21; 435/455; 435/368

(57) ABSTRACT

The present invention includes compositions, methods and kits for non-nuclear transfer reprogramming an adult differentiated cell obtained from an adult tissue into an ES-like cell. The reprogrammed cell can be converted into an ES-like cell which can be re- or trans-differentiated into various differentiated cell types. The present invention further relates to identification of a novel signaling pathway, and components thereof, which effect reprogramming of a cell. The present invention further comprises compositions, methods and kits for regulating the mammalian cell cycle and cellular proliferation, as well as for treating diseases and for identifying components that affect the cell cycle, and reprogram cells, among other things.

### BACKGROUND OF THE INVENTION

Regenerative medicine holds great promise as a therapy for many human ailments, but also entails some of the most difficult technical challenges and controversial ethical issues ever encountered in modern scientific research. The hope for regenerative medicine is based primarily upon two significant biological breakthroughs of the past decade: cloning Dolly the sheep (Wilmut et al., 1997, Nature 385:810-813) and culturing human embryonic stem (ES) cells (Thomsom et al., 1998, Science 282:1145-1147). Regenerative medicine, or therapeutic cloning, would combine these two milestones to create pluripotent stem cells that are genetically matched to a patient in order to generate personalized tissues that would combat the ravages of aging and disease without organ rejection or other complications that plague conventional transplant therapy.

The technical challenges to regenerative medicine include low cloning efficiency, a short supply of potentially pluripotent tissues, and a generalized lack of knowledge as to how to control cell differentiation and what types of ES cells can be used for selected therapies. Further, undifferentiated ES cells can form teratomas (benign tumors) containing a mixture of tissue types.

In an attempt to overcome the barriers to realizing the potential of regenerative medicine, especially with respect to human ES cells, many researchers have investigated new avenues for generating stem cells from tissues that are not of fetal origin. Such approaches often involve either the manipulation of autologous adult stem cells or methods to allow allogeneic stem cells to evade the recipient's immune system.

The advantage of using autologous adult stem cells for regenerative medicine lies in the fact that they are derived from and returned to the same patient, and are therefore not subject to immune-mediated rejection. The major drawback is that these cells lack the plasticity and pluripotency of ES cells and thus their potential is uncertain. To overcome this hurdle, much attention has been directed towards bone marrow cells, which can differentiate into such diverse tissues as bone, cartilage, and muscle (Pittenger et al., 1999, Science 284:143-147). It has also been shown that mouse bone marrow cells, when injected directly into infracted mouse hearts, can develop into myocytes and vascular structures (Orlic et al., 2001, Nature 410:701-705). Similar experiments have shown that mouse bone marrow cells delivered intravascularly are able to migrate to the central nervous system and eventually exhibit neuron-like phenotypes (Brazelton et al., 2000, Science 290:1775-1779; Mezey et al., 2000, Science 290:1779-1782).

The regenerative capacity of adult cells is not limited to those stem cells obtained derived from bone marrow. That is, neural stem cells are capable of differentiating into blood cells when transplanted into bone marrow (Bjornson et al., 1999, Science 283:534-537). However, although these results are promising, the number of pluripotent bone marrow and neural stem cells is limited, and the ability to express a few phenotypic characteristics of another cell type does not immediately provide a system that replaces damaged or diseased tissue. In addition, it has not been possible to generate ES-lines from adult humans. While this appears to be technically difficult, tremendous effort has been made since this approach would have immediate clinical applications, such as the generation of ES-lines from patients (e.g., cancer, cardiovascular diseases, and neurodegenerative diseases) which would provide cell-based therapy to cure these diseases.

Because of the numerous obstacles and technical difficulties in ES-cells and functional uncertainties in adult stem cells, many researchers are now launching enormous efforts to develop strategies to reprogram somatic cells from adult tissues to create pluripotent ES-like cells. This approach provides immediate clinical application, since pluripotent ES-like cell generated from adult patients matched to the intended recipient. In order to reprogram a mature cell, the technology to date has focused on fusing mature cells with embryonic germ cells-the technology termed as somatic-cell nuclear transfer (SCNT). After fusion, mature cell nuclei displayed pluripotent properties similar to that of the embryonic germ cells (Tada et al., 1997, EMBO J. 16:6510-6520). This process essentially returns the mature adult cell to an earlier developmental state (immature state) in which the cell can mature into any type of tissue. However, similar to other attempts to create pluripotent cells, such reprogramming methods have been hampered by the need for ES-cells or embryonic germ cells, and the ethical and religious issues that come along with using human embryos. In addition, there are numerous practical difficulties in SCNT including the short supply of human oocytes for SCNT.

The goal of cellular reprogramming method is to identify an agent, such as a compound, gene product, extracellular signal or ligand that when present or absent, can revert a mature cell to an ES-like pluripotent state. Although progress in this field has been slow, certain advances have been made. For instance, oligodendrocyte precursor cells (OPCs), which usually terminally differentiate into post-mitotic oligodendrocytes, were shown to revert to a state that resembles multipotential stem cells when OPCs were sequentially exposed to fetal calf serum and basic fibroblast growth factor (bFGF) (Kondo et al., 2000, Science 289:1754-1757).

These results are a promising first step in the field of regenerative medicine because they demonstrate that cells, albeit, precursor cells which are not mature adult cells, can be de-differentiated and reversed to a neural stem cell, but not to an ES-cell. While this is not a complete reversal, these results demonstrate that a cell can be de-differentiated, or reprogrammed, to a neural stem cell-like cells without the need for ES-cell or embryonic germ cells as starting material. Instead, non-ES or non-embryonic germ cells can be de-differentiated in the presence of extracellular signals and other signals. However, OPCs are in a precursor state themselves, and do not represent an abundant source of starting cells for use in regenerative medicine.

Further, despite limited success in de-differentiating OPCs, until now, no methods exist for complete reprogramming of a terminally differentiated adult cell into a stem cell-like cell. That is, to date, research in reprogramming cells has not led to the development of any method for converting a mature and abundant differentiated adult cell into a pluripotent stem-like cell. Moreover, there is no prior art method for re-differentiating or trans-differentiating a mature but reprogrammed cell into the same or another phenotypically and functionally different differentiated cell type of the same or different lineage.

There is great interest in methods for converting a specialized adult cell into a stem cell-like cell useful for, inter alia, cell-based therapy. In the search for reagents and/or compounds that may reprogram somatic adult cell (i.e., reprogramming factors and/or reagents that induce certain signaling pathways, growth factors, genes, and ligands, and other materials that can be used to reprogram mature adult cells), attention has recently turned to the potential use of non-toxic, non-cytopathic obligate intracellular microorganisms due to the way they modify, or simplify, the intracellular microenvironment of the adult cells in order to facilitate the long infection process or long-term survival of the organism within the intracellular milieu.

One such organism is Mycobacterium leprae, the causative agent of leprosy or Hansen's disease, which is an obligate intracellular bacterium. M. leprae has a unique ability to invade the Schwann cell, the glial cell of the peripheral nervous system (PNS), in addition to other cell types including skin cells. Remarkably, with a limited set of functional genes in its genome, this leprosy bacillus is highly adapted to long-term intracellular survival by subverting Schwann cell functions without causing apparent host cell death (Cole et al., 2001, Nature 409:1007-011; Rambukkana et al., 2001; Rambukkana et al., 2002, Science 296: 927-931; Brophy, 2002, Science 296:862-863).

M. leprae binds to α-dystroglycan on target cells via a laminin-2 intermediary molecule that deposits on the cell surface (Rambukkana et al., 1998, Science 282:2076-2079; Rambukkana et al., 1997, Cell 88: 811). α-dystroglycan is highly conserved and is present in the cell membrane, and plays a role in the physical interaction between the cellular basement membrane and the cytoskeleton. Laminin-2 is also conserved and is present in many cell types and plays a role in cell adhesion, migration and in development.

In the PNS, Schwann cells exist as two distinct phenotypes depending on the axons they associate with and on subsequent myelin synthesis. More particularly, Schwann cells that enclose small axons and larger axons develop into non-myelinated and myelinated Schwann cell-axon units, respectively. M. leprae attachment to myelinated and non-myelinated Schwann cell-axon units specifically induces demyelination and bacterial invasion, respectively (Rambukkana et al., 2002, Science 296: 927-931). Since it is the non-myelinated Schwann cells that are mostly susceptible to M. leprae invasion, and preferentially harbor M. leprae in vitro (Rambukkana et al., 2002, Science 296:927-931), and in are present in the majority of leprosy patients demonstrating high bacterial load (Shetty et al., 1988, J. Neurol. Sci. 88:115-131), the long-term intracellular survival of M. leprae within non-myelinated Schwann cells is the key to the persistent M. leprae infection in the PNS (Stoner, 1979; Mukherjee and Antia, 1986; Job, 1989). Subsequent to invasion, M. leprae reside within Schwann cells for a long period before causing neurological injuries.

Modulation of cellular microenvironment is likely to be a prerequisite for such long-term intracellular residence of this bacterium within human Schwann cells. One key to the pathogenic potential of long-term intracellular survival of M. leprae within human Schwann cells lies in the ability of this bacterium to propagate its intracellular niche so that sufficient Schwann cells are available for bacterial survival and replication (Rambukkana et al., 2002). One effective way of propagating cellular niche lies within the capacity of intracellular M. leprae to induce Schwann cell proliferation (Rambukkana et al., 2002; Rambukkana et al., 2004, Curr. Opinion Immunol. 16:511-518).

Due to its interaction with α-dystroglycan and laminin-2 molecules, which are present on a wide plethora of all types, M. leprae can potentially infect many cell types (see, e.g., Spear, 1998, Science 282:1999-2000). However, M. leprae replicate at a temperature of about 27° to 33° C., such that the natural infection process is limited to peripheral nerves and skin tissues, which tend to be cooler than core body temperatures. Thus, other than the temperature limitation, M. leprae can be used to infect a wide spectrum of cell types. In addition, other invasive mechanisms appear to be involved in the M. leprae entry process and such mechanisms are likely present in many cell types, since M. leprae infects a wide range of cell types.

While limited progress has been made towards developing methods to reprogram cells without using scarce and controversial embryonic stem or germ cells, there remains a long-felt need for methods for producing a stem cell-like cell using a mature adult cell, which stem cell-like cell can be used in cell-based therapies to, e.g., regenerate or replace organs. Accordingly, there exists a long-felt need for development of methods for producing stem cell-like progenitor cells for use in regenerative medicine, especially methods that comprise the use of defined reagents and which do not require either embryonic stem cells/germ cells or adult stem cells, which need extremely long-term procedures for manipulation the cells to be functional and have uncertain potential. The present invention meets this need.

### BRIEF SUMMARY OF THE INVENTION

The invention includes a method of producing a embryonic stem-cell like cell (ES-like cell) by non-nuclear transfer reprogramming of adult differentiated cell. The method comprises contacting a differentiated adult cell with a Mycobacterium leprae bacterium, or a component thereof, thereby producing the reprogrammed ES-like cell.

The invention includes a cell produced by this method.

In one aspect, the bacterium is viable or non-viable.

In another aspect, the adult differentiated cell is a eukaryotic cell.

In yet another aspect, the eukaryotic cell is a mammalian cell.

In a further aspect, the mammalian cell is selected from the group consisting of a Schwann cell, a keratinocyte, a beta-islet cell, a hepatocyte, and a heart muscle cell.

In another aspect, the cell is a Schwann cell.

In yet another aspect, the differentiated cell is incubated with the bacterium, or the component thereof, for at least 15 to about 30 days.

In a further aspect, the component is at least one component selected from the group consisting of a PGL-1, a whole cell wall fraction, and a cell wall protein, a cell wall lipid, a cell wall carbohydrate, a protein released from a viable bacterium, and a protein secreted from a viable bacterium.

In one aspect, the method further comprises converting the reprogrammed cell to a stem cell-like cell, wherein the conversion comprises contacting the reprogrammed cell with a progenitor medium, thereby converting the reprogrammed cell to a stem cell-like cell. The invention includes a cell produced by this method.

The invention includes an isolated ES-like cell, wherein the cell is produced by contacting an adult differentiated cell with a Mycobacterium leprae bacterium, or a component thereof.

In one aspect, the bacterium is selected from the group consisting of a viable bacterium and an irradiated bacterium.

The invention also includes a method of producing a re-differentiated cell. The method comprises: (a) contacting a differentiated adult cell with a Mycobacterium leprae bacterium, or a component thereof, thereby producing a reprogrammed ES-like cell; (b) incubating the reprogrammed ES-like cell in a progenitor cell medium, thereby producing a stem cell-like cell and; (c) re-differentiating the stem cell-like cell into a differentiated cell of the same cell type as the differentiated adult cell, thereby producing a re-differentiated cell.

The invention includes a re-differentiated cell produced using the method.

In one aspect, the differentiated adult cell is an adult human Schwann cell, and wherein the re-differentiated cell is a neural-like cell.

In another aspect, the neural-like cell is selected from the group consisting of a neuron-like cell and an oligodendrocyte-like cell.

In a further aspect, the stem cell-like cell of step (b) is grown to produce at least two stem cell-like cells prior to the re-differentiation of (c).

The invention includes a method of producing a trans-differentiated cell. The method comprises: (a) contacting a differentiated adult cell with a Mycobacterium leprae bacterium, or a component thereof, thereby producing a reprogrammed ES-like cell; (b) incubating the reprogrammed ES-like cell in a progenitor cell medium, thereby producing a stem cell-like cell and; (c) trans-differentiating the stem cell-like cell into a differentiated cell of a different cell lineage than the differentiated adult cell, thereby producing a trans-differentiated cell.

The invention includes a trans-differentiated cell produced using this method.

In one aspect, the stem cell-like cell of step (b) is grown to produce at least two stem cell-like cells prior to the trans-differentiation of (c).

The invention includes a method of producing a neurosphere. The method comprises contacting a differentiated adult Schwann cell with a Mycobacterium leprae bacterium, or a component thereof, thereby reprogramming the Schwann cell to produce an ES-like cell, and incubating the reprogrammed ES-like cell in a progenitor medium, thereby producing a neurosphere.

The invention includes an isolated neurosphere produced by this method.

The invention also includes a method of producing a neuron-like cell. The method comprises contacting a differentiated adult Schwann cell with a Mycobacterium leprae bacterium, or a component thereof, thereby reprogramming the Schwann cell into an ES-like cell, incubating the reprogrammed ES-like cell in a progenitor medium to produce a neurosphere, and incubating the neurosphere in a neuronal differentiation medium, thereby producing a neuron-like cell.

The invention includes a neuron-like cell produced using this method.

The invention includes a method of producing an oligodendrocyte-like cell. The method comprises contacting a differentiated adult Schwann cell with a Mycobacterium leprae bacterium, or a component thereof, thereby reprogramming the Schwann cell into an ES-like cell, incubating the reprogrammed ES-like cell in a progenitor medium to produce a neurosphere, and incubating the neurosphere in an oligodendrocyte differentiation medium, thereby producing an oligodendrocyte-like cell.

The invention includes an oligodendrocyte-like cell produced using this method.

The invention includes a method of affecting the passage of a cell through the cell cycle. The method comprises contacting a cell with a Mycobacterium leprae bacterium, or a component thereof, thereby affecting the passage of the cell through the cell cycle.

The invention includes a method of increasing the level of cyclin D1 expression in a cell. The method comprises contacting a cell with a Mycobacterium leprae bacterium, or component thereof, thereby increasing the level of cyclin D1 expression in the cell.

The invention includes a method of inducing MEK/Pi3K-independent phosphorylation of Erk1/2 in a cell. The method comprises contacting a cell with a Mycobacterium leprae bacterium, or component thereof, thereby inducing MEK/Pi3K-independent phosphorylation of Erk1/2 in the cell.

In one aspect, the method comprises phosphorylating the serine 158 amino acid residue of Lck.

The invention includes a method of inducing MEK/Pi3K-independent phosphorylation of GSK3β in a cell, the method comprising contacting a cell with a Mycobacterium leprae bacterium, or component thereof, thereby inducing MEK/Pi3K-independent phosphorylation of GSK3β in the cell.

In one aspect, the method comprises phosphorylating the serine 158 amino acid residue of Lck.

The invention also includes a method of identifying a component a Mycobacterium leprae bacterium that reprograms a differentiated cell. The method comprises contacting a differentiated adult cell with a component of a Mycobacterium leprae bacterium, and comparing the level of a marker of an undifferentiated state in the cell contacted with the component with the level of the marker in an otherwise identical cell not contacted with the component, wherein a higher level the marker in the cell contacted with the component compared with the level of the marker in the cell not contacted with the component is an indication that the component reprogrammed the cell, thereby identifying a component a Mycobacterium leprae bacterium that reprograms a differentiated cell.

The invention includes a component identified by this method.

In one aspect, the marker is selected from the group consisting of the level of cyclin D1 expression in a cell, the level of p21 expression in a cell, the level of phosphorylation of Lck at serine residue number 158, the level of proliferation without transformation, the level of MEK/Pi3K-independent Erk1/2 phosphorylation, the level of MEK/Pi3K-independent GSK3β phosphorylation, expression of an embryonic-stage marker protein, and expression of an embryonic-stage gene.

The invention includes a method of identifying a component of a Mycobacterium leprae bacterium that affects progression of a cell through the cell cycle. The method comprises contacting a cell with a component of a Mycobacterium leprae bacterium, and comparing the progression of the cell through the cell cycle with the progression through the cell cycle by an otherwise identical cell not contacted with the component, wherein a faster progression through the cell cycle by the cell contacted with the component compared with the progression through the cell cycle by the cell not contacted with the component is an indication that the component affects progression of a cell through the cell cycle, thereby identifying a component of a Mycobacterium leprae bacterium that affects progression of a cell through the cell cycle. The invention includes a component identified by this method.

The invention includes a method of identifying a component of a Mycobacterium leprae bacterium that increases cell proliferation. The method comprises contacting a cell with a component of a Mycobacterium leprae bacterium, and comparing the level of proliferation of the cell with the level of proliferation of an otherwise identical cell not contacted with the component, wherein a greater level of proliferation of the cell contacted with the component compared with the level of proliferation of the otherwise identical cell not contacted with the component is an indication that the component increases cell proliferation, thereby identifying a component of a Mycobacterium leprae bacterium that increases cell proliferation.

The invention includes a component identified by this method.

The invention includes a method of treating a neurological disease in an animal in need thereof. The method comprises administering to the animal an effective amount of an ES-like cell produced by a method of producing a reprogrammed embryonic stem-cell like cell (ES-like cell) comprising contacting a differentiated adult cell with a Mycobacterium leprae bacterium, or a component thereof, thereby producing the reprogrammed ES-like cell, thereby treating a neurological disease in the animal.

In one aspect, the neurological disease is selected from the group consisting of Alzheimer's disease, Parkinson's disease, central nervous system injury, and multiple sclerosis.

In another aspect, the central nervous system injury is selected from the group consisting of spinal cord injury, stroke, ischemia, and brain injury.

The invention includes a method of treating a neurological disease in an animal in need thereof. The method comprising administering to the animal an effective amount of a neuron-like cell produced by a method comprising contacting a differentiated adult Schwann cell with a Mycobacterium leprae bacterium, or a component thereof, thereby reprogramming the Schwann cell into an ES-like cell, incubating the reprogrammed ES-like cell in a progenitor medium to produce a neurosphere, and incubating the neurosphere in a neuronal differentiation medium, thereby producing a neuron-like cell, thereby treating a neurological disease in the animal.

The invention includes a method of treating a neurological disease in an animal in need thereof. The method comprises administering to the animal an effective amount of a oligodendrocyte-like cell produced by a method comprising contacting a differentiated adult Schwann cell with a Mycobacterium leprae bacterium, or a component thereof, thereby reprogramming the Schwann cell into an ES-like cell, incubating the reprogrammed ES-like cell in a progenitor medium to produce a neurosphere, and incubating the neurosphere in an oligodendrocyte differentiation medium, thereby producing an oligodendrocyte-like cell, thereby treating a neurological disease in the animal.

The invention includes a kit for reprogramming an adult differentiated cell. The kit comprises an irradiated Mycobacterium leprae bacterium, or a component thereof, and the kit further comprises an applicator and an instructional material for use of the kit.

In one aspect, the kit further comprises a progenitor medium.

In another aspect, the kit further comprises a differentiation medium.

In a further aspect, the component is a cell wall obtained from a Mycobacterium leprae bacterium.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A through FIG. 1G, is an image demonstrating that intracellular M. leprae induced proliferation of adult human primary Schwann cells. FIG. 1A is a graph depicting purification of Schwann cells from peripheral nerve tissues from human organ donors by cell sorting using FACSVantage dual-laser flow cytometer with anti-p75 MAb.

FIG. 1B is an image of a photomicrograph depicting that propagated cells (passage number 3) express non-myelinating Schwann cell marker p75 and GAP43 (as shown in the inset) as determined by immunofluorescence and counterstained with DAPI. Labeling can be seen in both membrane and in the entire cytoplasm.

FIG. 1C is an image of a photomicrograph depicting intracellular localization of M. leprae as determined by double immunolabeling after 15 days (ML/15 days) of infection with antibodies to Schwann cell-specific S-100 (labeling can be seen in the entire cytoplasm) and M. leprae-specific PGL-1 (labeling can be seen as rod-shaped structures in the cytoplasm).

FIG. 1D is an image of a photomicrograph depicting PGL-1 labeling of 30 day-infected Schwann cells (ML/30 days) counterstained with DAPI (shown at the upper left corner of the image). Ultrastructural studies of 30 day-infected Schwann cells depicting M. leprae at different locations within the cytoplasm as demonstrated by both upper right and bottom panels of the image.

FIG. 1E is an image of immunoblots of Schwann cell lysates prepared from Schwann cells either with (at day 7 and 30) and without (control) M. leprae infection as indicated along the bottom edge of the figure by the lane designations of “Control”, “ML/7 days”, “ML/30 days.” The immunoblots were stained with antibodies to cleaved caspace-3 (top panel), caspase-9 (middle panel), and activated PARP (bottom panel). Also depicted is a lane demonstrating immunostaining of lysates from Camptothecin (1.5 μM)-treated Schwann cells as a positive control for the induction of apoptosis.

FIG. 1F is an image of a photomicrograph depicting that human Schwann cells harboring intracellular M. leprae (FIG. 1F2, ML/BrdU) exhibit an increased BrdU uptake as compared with non-infected control Schwann cells (FIG. 1F2, Control/BrdU). FIG. 1G depicts a graph demonstrating cell cycle FACS analysis of M. leprae-infected Schwann cells (*: p<0.005; **: p<0.001) demonstrating the increased percentage of S phase cells in M. leprae-infected cells (ML) as compared with non-infected (Control) cells at both 15 and 30 days post-infection (p.i.), where the difference in percent of S phase cells between infected and non-infected Schwann cells is even greater at 30 days p.i. than at 15 days. The data are presented as a percentage of total S phase cells from three independent experiments.

FIG. 2, comprising FIGS. 2A through 2E, depicts differentially expressed genes in M. leprae-infected human Schwann cells after 30 days as analyzed by Affymetrix human gene chips. Genearray analysis of human Schwann cells 30 days after M. leprae infection was analyzed using Affymetrix human gene chips. FIG. 2A is a graph depicting the total number of human genes in the genechip, total number of genes expressed in human Schwann cells, and the total number of differentially expressed up-regulated and down-regulated genes 30 days after M. leprae infection. FIG. 2B is a graph depicting the major functional categories of gene clusters differentially expressed in response to intracellular M. leprae.

FIG. 2A depicts differentially expressed genes after 30 days of infection clustered according to functional annotations and the total number of apoptosis- and cell cycle-related genes is also presented. Note the highest number of gene expression in 30 day-infected Schwann cells. FIG. 2B is a graph depicting the expression of major functional gene clusters in human Schwann cells in response to intracellular M. leprae.

FIGS. 2C and 2D show total number of differentially expressed genes in human Schwann cells infected with viable M. leprae and the gene expression profiles depicted in FIG. 2E were obtained using human Schwann cells infected with irradiated M. leprae. Purified primary Schwann cells from two different organ donors were infected for 30 days with viable M. leprae (three separate genearray experiments; Exp. #1 through #3) and irradiated M. leprae (one genearray experiment; Exp. # 4). Differentially expressed genes for apoptosis and cell cycle were clustered, and presented as percentages (the percentage of differentially expressed genes show only a minor fluctuation, about ±2%) in all four experiments). The key to the graphs is as indicated: 1. anti-apoptosis; 2. cell cycle; 3. growth factors; 4. transcription factors; 5. signaling; 6. metabolism; 7. unknown; 8. myelin/ECM/ECMR*, where * indicates down-regulated genes, all other bars represent genes that were up-regulated.

FIG. 3, comprising FIGS. 3A and 3B, is a series of graphs depicting the expression of transcription, growth, and neurotrophic related genes in human Schwann cells in response to intracellular M. leprae. FIG. 3A depicts the expression of transcription related genes in Schwann cell cultures at 3 days (open bar), 5 days (middle solid bar), and 30 days (right-most solid bar) post infection. FIG. 3B depicts the fold increase in expression of various growth and neurotrophic factors and growth factor receptors in Schwann cells infected with M. leprae.

FIG. 4 is a graph depicting the increased expression of developmental/embryonic genes, including HOX genes, induced in human Schwann cells in response to intracellular M. leprae 30 days after infection.

FIG. 5 is a graph depicting the significant down regulation of Schwann cell myelin genes and differentiation-associated genes, such as genes for extracellular matrix proteins and their receptors, by intracellular M. leprae.

FIG. 6, comprising FIGS. 6A through 6G, is a series of images depicting the loss of the differentiated markers at protein levels in human Schwann cells infected with M. leprae. FIG. 6A is an image of an immunoblot depicting ErbB3 protein expression in human Schwann cells 30 days after M. leprae infection. FIG. 6B is an image depicting ErbB3 protein expression in rat dorsal root ganglion (DRG) explant cultures 25 days after M. leprae infection. FIGS. 6C through 6F are images depicting immunofluorescence of rat DRG cultures 25 days after infection with M. leprae. Uninfected cells produce compact myelin (FIG. 6C; shown as intact myelin segments along axons), as demonstrated by staining with anti-myelin basic protein (MBP) antibodies whereas M. leprae infected DRG cells do not form compact myelin (FIG. 6D; myelin sheaths are absent in this figure), suggesting M. leprae down-regulate differentiation of Schwann cells at protein levels. Nuclear labeling was almost identical between infected and non-infected cultures (FIGS. 6E and 6F, respectively; DAPI labeling is indistinguishable from control and equal number of healthy nuclei can be seen in both control and infected cells). FIG. 6G is an image of an immunoblot of total DRG culture lysates probed using anti-P0 myelin protein demonstrating that M. leprae infection substantially reduces the expression of the myelin-related protein P0 in infected cells.

FIG. 7, comprising FIGS. 7A through 7E, demonstrate that intracellular M. leprae accelerated G1 phase progression and induced continuous proliferation of human Schwann cells without causing transformation. FIG. 7A is a graph depicting cell cycle distribution of asynchronously growing human Schwann cells that harbored intracellular M. leprae for 15 and 30 days as analyzed by FACScan using propidium iodide (PI). The percentage of cells in G1, S and G2 phases are shown. The data presented were obtained from three separate experiments from infected and non-infected Schwann cells purified from two different organ donors (*: p<0.005; **: p<0.001).

FIG. 7B is a graph depicting that overexpression of the T156A cyclin D1 dominant negative mutant (DNM) decreased the G1 phase progression of M. leprae infected Schwann cells. The data depict cell cycle FACS analysis of 30 day-infected human primary Schwann cells transfected with vector alone and T156A/cyclin D1 DNM. Data from three independent cell cycle FACS experiments showing the mean percentage of cell population in G1, S and G2 phases (*: p<0.005).

FIG. 7C is a graph depicting continuous Schwann cell proliferation induced by intracellular M. leprae leads to cell propagation but does not cause cellular transformation. Total Schwann cell count from asynchronized cultures before (day 0; start with equal cell numbers) and 7, 15, and 30 days after M. leprae infection is shown from four individual experiments (**: p<0.0005).

FIG. 7D is an image depicting tissue culture plates demonstrating transformation assays of 30 day-M. leprae infected and non-infected Schwann cells and SK-BR-3 breast cancer cells (as a positive control) showing no plaque formation in infected Schwann cells (Schwann cells/+ML), no plaque formation in non-infected control cells (Schwann cells/-ML), compared with numerous plaques in SKBR-3 cancer cells, as indicated.

FIG. 7E is an image depicting immunoblots of cell lysates demonstrating strong expression of caveolin-1, a protein that disappears (cannot be detected) in transformed cells and in cancer cells (Galbiati et al., 1998, EMBO J. 17:6633-6648), in both control human Schwann cells (hSC/30 days) and 30 day-infected human Schwann cells (ML+hSC/30 days), compared with SKBR-3 cells and SV-40-transformed human Schwann cell line (SV40-hSC) where little calveolin-1 was detected.

FIG. 8 is a schematic representation demonstrating the significant overlap between genes expressed in M. leprae-reprogrammed adult human Schwann cells compared with signature stem cell genes expressed in mouse neural stem cells, and mouse embryonic stem cell-specific genes.

FIG. 9, comprising FIGS. 9A and 9B, is a series of images demonstrating that reprogrammed human adult Schwann cells develop into stem cell-like cells. More specifically, the images depict the formation of primary neurospheres from reprogrammed Schwann cells infected with M. leprae for 30 days and cultured in progenitor media comprising bFGF without serum. FIG. 9A, comprising FIG. 9A-1 through FIG. 9A-5, depicts a series of images depicting different stages of the development of a neurosphere from a single cell (arrows). FIG. 9B, comprising FIG. 9B-1 through FIG. 9B-5, depicts a series of images showing the dissociated full-grown neurosphere and the formation of second neurosphere from the dissociated first neurosphere. FIG. 9B-5 depicts, in an inset image at the bottom left corner of the image, labeling of the neurosphere using an antibody to nestin, a neural stem cell marker.

FIG. 10, comprising FIGS. 10A and 10B, depicts images showing light microscopy and immunofluorescence analyses of human adult primary Schwann cells before (A) and after reprogramming using irradiated M. leprae with intact cell walls for 30 days followed by incubation with progenitor media (B). FIG. 10A, comprising FIG. 10A-1 (top) and FIG. 10A-2 (bottom), is an image depicting a phase contrast image (FIG. 10A-1) and cells labeled with adult Schwann cell marker S-100 antibody (FIG. 10A-2).

FIG. 10B, comprising FIG. 10B-1 through FIG. 10B-5, depicts a series of images depicting different stages of neurosphere formation obtained from a single M. leprae reprogrammed cell after incubating for 10-15 days using bFGF-containing media. Note the development of neurospheres from a single cell; 10B-1, single cell; 10B-2, 3 cell stage; 10B-3, several cells and 10B-5 represents a mature sphere. FIG. 10B-4 demonstrates that the primary neurospheres are positive for nestin, the neural stem-cell marker, as analyzed by immunofluorescence with anti-nestin antibody and the cell is depicted using light microscopy in FIG. 10B-5.

FIG. 11, comprising FIGS. 11A through 11K, is a series of phase contrast images depicting the different stages of differentiation of dissociated neurospheres to neuron-like cells in the presence of neuronal differentiation media. FIG. 11A through 11G show early stage of neuronal differentiation from dissociated spheres. FIG. 11H through FIG. 11K depict images showing mature neuron-like cells with different morphology differentiated from several secondary neurospheres.

FIG. 12, comprising FIG. 12A through FIG. 12D, is a series of images depicting the striking morphological similarities in neuron-like cell development derived from neurospheres from reprogrammed human adult Schwann cells and the differentiation of a mature neuron from an undifferentiated neural progenitor as depicted in a text by Nobel Laureate Santiago Ramon y Cajal (1937, In: Recollection of My Life, translated by E. H. Craigie and J. Cano, Amer. Phil. Soc., Philadelphia, Pa., reprinted in 1989 by MIT Press, Cambridge, Mass.).

FIG. 13, comprising FIGS. 13A through 13N, is a series of images depicting the morphological and cell-marker changes between adult human Schwann cells, neurospheres derived from reprogrammed Schwann cells and neuron-like cells differentiated from secondary neurospheres. FIG. 13A is an image depicting a light microscopy view of adult primary human Schwann cells, FIG. 13B is an image depicting immunofluorescence of adult primary human Schwann cells expressing the prototypical Schwann cell marker S-100 (all cells were positive for S-100 antibody). FIGS. 13C through 13G are images depicting primary neurospheres derived from human primary Schwann cells after a 30 day incubation with irradiated M. leprae followed by culturing in medium comprising bFGF. FIG. 13H is an image of a neurosphere derived from a human primary Schwann cell incubated with irradiated M. leprae and stained for the neural stem cell marker nestin. FIGS. 13I and 13J are phase contrast images depicting the differentiation of neurospheres in neuronal differentiation medium comprising NT3. FIG. 13K is an image depicting the immunofluorescence of differentiating neuron-like cells showing the increasing expression of the early neuron marker MAP2 and the decreasing expression of the Schwann cell marker S-100. FIG. 13L is an image depicting the diffuse staining of the neural crest cell-specific marker MAP2 on a neuron-like cell derived from neurospheres that are cultured in media comprising NT3. FIGS. 13M and 13N are a series of images depicting the relative paucity of S-100 staining in differentiated neuron-like cells compared to the abundant and diffuse staining of MAP2 in differentiated neuron-like cells differentiated/derived from neurospheres from reprogrammed M. leprae infected Schwann cells.

FIG. 14, comprising FIG. 14A through FIG. 14E, is a series of images depicting the differentiation of human neurospheres derived from reprogrammed M. leprae infected human primary Schwann cells to MAP2 positive neuron-like cells. FIG. 14A is an image depicting a light microscopy view of a neurosphere differentiating to a neuron-like cell at an early stage. FIG. 14B is an image depicting the immunofluorescence of a neuron-like cell demonstrating the abundance of neural crest cell/developing neuron-specific MAP2 staining compared to the elimination of Schwann cell-specific S-100 staining (S-100 labeling could be seen in only in very few cells that are also positive for MAP2, suggesting that these remaining S-100 positive cells are in the process of changing their cell fate). FIGS. 14C and 14D depict the acquisition of the MAP2 phenotype in neuron-like cells and the complete elimination of Schwann cell marker S-100 on the surface of neuron-like cells, respectively. FIG. 14E is an image depicting that neuron-like cells that are further incubated in neuronal media express the MAP2 phenotype while no longer exhibiting the mature Schwann cell-specific S-100 phenotype. Also note the synaptic formation of synaptic-like structures in MAP2 positive neuron-like cells.

FIG. 15, comprising FIGS. 15A through 15D, is a series of images depicting the acquisition of markers specific for mature neurons in neuron-like cells differentiated from neurospheres derived from reprogrammed human primary Schwann cells incubated with M. leprae. FIG. 15A is an image depicting a light microscopy view of a differentiated neuron like cell. FIG. 15B is an image of a neurosphere derived from reprogrammed Schwann cell to produce a neuron-like cell stained for the mature neuron-specific marker neurofilament 200 kDa and the Schwann cell marker S-100. FIG. 15C is an image depicting neuron-specific β-tubulin III (TUJ1) staining on neuron-like cells. FIG. 15D is an image depicting the diffuse and intense staining of the neuron-specific marker neurofilament-L on neuron-like cell differentiated from neurosphere derived from reprogrammed Schwann cells in comparison to the very light, almost absent, staining for the Schwann cell-specific marker P-75 (p75) on the same cells. Please note that p75 is present in both immature and mature Schwann cells, and the faint labeling is likely due to immature phenotype. All neuron-like cells differentiated from neurospheres derived from reprogrammed cells are strongly labeled with indicated neuron-specific marker antibodies with filamentous appearance except NeuN which strongly labeled nuclei in addition to dendrites. All neuron-like labeling observed resembles the labeling of neurons derived from adult brain or peripheral nerve.

FIG. 16, comprising FIGS. 16A through 16F, is a series of images depicting the acquisition of CNS oligodendrocyte-specific marker O4 in oligodendrocyte-like cells differentiated from neurospheres derived from reprogrammed human primary adult Schwann cells incubated with M. leprae in response to oligodendrocyte media containing PDGF. FIGS. 16A-E are phase contrast images showing different stages of development of oligodendrocyte-like cells from a single neurosphere, and FIG. 16F depicts immunofluorescence image showing immuno-doublelabeling with both anti-O4 antibody and anti-GFAP antibody, which antibodies recognize oligodendrocytes (O4) and astrocytes/Schwann cells (GFAP), respectively. Only O4 positivity indicates neurospheres from reprogrammed cells differentiated to CNS oligodendrocyte-like cells.

FIG. 17 is a diagram illustrating, without wishing to be bound by any particular theory, the steps involving in generation of stem cells from human adult Schwann cells: (I) reprogramming, (II) conversion of reprogrammed cells to a stem cell-like cell phenotype (e.g., neurospheres); and (III) re-differentiation of a stem cell-like cell into a differentiated cell (e.g., neurospheres to neuron-like cells) thereby demonstrating acquisition of highly differentiated stage, e.g. neuron.

FIG. 18, comprising FIGS. 18A and 18B, is a series of graphs depicting analysis of gene array data demonstrating activation of human primary Schwann cell cycle genes by intracellular M. leprae post-30 day infection. FIG. 18A is a bar graph demonstrating that the key regulatory genes of the G1 phase (i.e., cyclin D1, cyclin D2, Rb1, Rb2) and G2 phase (i.e., cyclin A, cyclin B, cdc2, cdc25, and the like) were upregulated in M. leprae infected human Schwann cells. FIG. 18B is a graph depicting the normalized down regulated expression of G1 phase cell cycle inhibitor genes (i.e., p57, p21, p16, p18, and p23) in M. leprae infected human Schwann cells. Fold difference in gene expression was graphed after normalization using the respective gene expression in control cells (uninfected samples).

FIG. 19, comprising FIGS. 19A through 19F, demonstrates that intracellular M. leprae regulates cyclin D1 nuclear accumulation and S-phase entry by activation of Erk1/2 via MEK/Pi3-kinase-independent pathways. FIG. 19A is an image depicting Western blot analysis of total lysates of human Schwann cells from two different organ-donors that had been infected with M. leprae for 30 days and synchronized for 48 hours. The total lysate proteins were then blotted and stained using a panel of phospho-specific antibodies as indicated (pErk 1/2, pAkt, pGSK-3β, β-Catenin, β-actin). β-actin labeling is shown as an internal standard to demonstrate that equal amounts of proteins were loaded in each lane.

FIG. 19B is an image depicting Western blot analysis of 30 day-infected and non-infected Schwann cells synchronized for 48 hours in the continuous presence of MEK1/2 inhibitor (MEKI) UO126 and PI3k inhibitor (Pi3KI) LY294002.

FIG. 19C is an image depicting Western blot analysis of 30 day-infected and non-infected Schwann cells synchronized for 48 hours in the continuous presence of SOS inhibitor. For both Western blot analyses depicted in FIGS. 19B and 19C, total lysates of Schwann cells were blotted with phospho-Erk1/2 and phospho-GSK-3β antibodies. Note that inhibitors exert no effect on Erk1/2 and GSK-3β phosphorylation.

FIG. 19D is an image depicting Western blot analysis depicting that human Schwann cells that harbor intracellular M. leprae show increased Erk1/2 kinase activity in a MEK/Pi3-kinase-independent manner. Active phospho-Erk1/2 was immunoprecipitated from lysates of synchronized human Schwann cells using anti-phospho Erk1/2 (p42/44 MAPK) antibody. The resulting immunoprecipitate was then incubated with a fusion protein of Elk-1 transcription factor and phosphorylation of Elk-1 at Ser 383, a major phosphorylation site of Erk1/2, was determined by phosho-Elk-1 antibody as a measure of kinase activity.

FIG. 19E is an image of a Western blot demonstrating that cyclin D1 nuclear accumulation by intracellular M. leprae is mediated by MEK- and PI3k-independent phosphorylation of Erk1/2. Nuclear protein extracts from synchronized human Schwann cells in the presence of MEK and Pi3-k inhibitors were blotted and reacted with antibody to cyclin D1, showing increased nuclear cyclin D1 in infected cells.

FIG. 19F is a graph depicting Erk2 dominant-negative (D-N) mutant (termed T192A/Erk2 D-N mutant) inhibits M. leprae-induced S-phase entry. Infected human Schwann cells were transfected transiently with a p44mapk D-N mutant (T192A) and vector alone and cell-cycle kinetics were analyzed using FACS (*: p<0.0001).

FIG. 20 is an image depicting, without wishing to be bound by any particular theory, a diagram describing the novel signal pathway (NSP) that regulates human Schwann cell proliferation, and possible role of this novel signaling pathway in regulating cell proliferation without transformation and reprogramming adult Schwann cells to stem cell-like cells.

FIG. 21, comprising FIGS. 21A through 21E, demonstrates cell cycle-related gene expression profiles of human primary Schwann cells in response to intracellular M. leprae. FIG. 21A is a graph demonstrating differentially expressed cell cycle-related genes in M. leprae-infected human Schwann cells as analyzed by Affymetrix human gene chips. FIG. 21A is a graph depicting that differentially expressed genes after 3, 7 and 30 days of infection clustered according to functional annotations and the total number of apoptosis- and cell cycle-related genes is presented.

FIG. 21B is a graph depicting differentially expressed genes using purified primary Schwann cells from two different organ donors infected for 30 days with viable M. leprae (three separate genearray experiments; Experiment numbers 1 to 3) and irradiated M. leprae (Experiment number 4). Differentially expressed genes for apoptosis and cell cycle were clustered, and presented as percentages (the percentage of differentially expressed genes show only a minor fluctuation, i.e., approximately ±2%, in all four experiments).

FIG. 21C is a graph depicting Cyclin D1 and p21 gene expression in human Schwann cells from two different donors infected with viable M. leprae for 30 days (p<0.001).

FIG. 21D is a graph depicting Cyclin D1 and p21 gene expression in human Schwann cells from two different donors infected with irradiated M. leprae (D) for 30 days (p<0.001).

FIG. 21E is a graph depicting quantitative Real-Time PCR analysis of cell cycle genes (cyclin D1, Rb1, p57, p16, and p21) in human Schwann cells after 7 (dark gray) and 30 (black) days of M. leprae infection.

FIG. 22, comprising FIGS. 22A through 22D, demonstrate long-term residence of intracellular M. leprae induced nuclear accumulation of cyclin D1 protein. FIG. 22A is an image of a Western blot depicting protein levels of cyclin D1 from 30 day-asynchronized cultures of human primary Schwann cells infected with M. leprae for 7, 15, or 30 days, and control Schwann cells that were not infected but grown under otherwise identical conditions as indicated along the bottom edge of the figure. FIG. 22 depicts Western blots of total cell lysates stained with antibodies to cyclin D1 and β-actin (as an internal control to confirm gel loading). Note the upregulation of cyclin D1 after 30 days.

FIG. 22B is an image of Western blots (two top panels) and Coomassie Blue stained gels (bottom two panels) depicting increased nuclear accumulation of cyclin D1 in M. leprae infected Schwann cells. FIG. 22B depicts results obtained using 30 day-infected and control cultures synchronized for 48 hours and then released to re-enter the cell cycle by the addition of 10% serum. Nuclear extracts were immunobloted with cyclin D1 antibody (top two panels). Equal amount of protein loading was verified by Coomassie-Blue staining (bottom two panels).

FIG. 22C is an image of a photomicrograph depicting immunofluorescence localization of cyclin D1 in synchronized control cells 15 hours after serum release.

FIG. 22D is an image of a photomicrograph depicting immunofluorescence localization of cyclin D1 in synchronized infected (30 days) cells 15 hours after serum release. FIGS. 22C and 22D depict the immunofluorescent staining of the cells used to produce the nuclear extracts analyzed as shown in FIG. 22B.

FIG. 23, comprising FIGS. 23A through 23I, is an image depicting that M. leprae-induced phospho Lck/Ser-158 in human Schwann cells serves as a direct activator of Erk1/2 phosphorylation via MEK1/2-independent and PKCα/βII-dependent pathway and MEK/Pi3-kinase-independent Erk1/2 phosphorylation induced by intracellular M. leprae in human primary Schwann cells depends on PKC activation. FIG. 23A is an image depicting a Western blot analysis of Schwann cells infected for 30 days (ML/30 days) were serum starved for 48 hours in the continuous presence of Pi3-kinase- and MEK-inhibitors with or without pan-PKC inhibitor bisindolylmaleimide, and the immunoblots of the total lysates of these cells were reacted with phospho-specific antibody to Erk1/2. β-actin labeling is shown to indicate the equal amount of proteins in each lane.

FIG. 23B is an image of a Western blot analysis depicting identification of PKCα/βII as the major phosphorylated PKC isoform in human Schwann cells and its activation by intracellular M. leprae. Lysates of serum-starved control and infected Schwann cells in the presence and absence of pan-PKC inhibitor were blotted and reacted with phospho-specific antibodies to different PKC isoforms and Erk1/2.

FIG. 23C demonstrates that PKCα/βII-dependent Erk1/2 phosphorylation in Schwann cells is mediated by Lck. MEK/Pi3K-independent Erk1/2 phosphorylation in the lysates of serum-starved infected Schwann cells in the presence and absence of Lck-inhibitors.

FIG. 23D is an image of a gel demonstrating expression of Lck in human Schwann cells and peripheral nerves. RT-PCR amplification of the human Lck gene in total RNA samples from control and 30 day-infected human primary Schwann cells is depicted. Actin (β-actin), a housekeeping gene, was used as an internal control to demonstrate gel loading. FIG. 23E is an image depicting a Western blot demonstrating total Lck protein expression in control and infected human Schwann cells from two different organ donors (left panel) and in human Sciatic nerve (right panel).

FIG. 23F is a diagram schematically depicting, without wishing to be bound by any particular theory, the domain structure and active versus inactive configurations of a Lck molecule. The left side of the diagram shows the inactive (or ‘closed’) confirmation in which the SH2 domain interacts with phosphorylated Tyr505. The right side of the diagram shows displacement of the intramolecular interactions and dephosphorylation of Tyr505 in active (or “open”) form (Kabouridis, 2003, Biochem. J. 371:907-915; Salter and Kalia, 2004, Nature Rev. Neurosci. 5:317-328). N and C denote N-terminus and C-terminus, which support membrane anchoring and inactive conformation by phosphorylating Tyr 505, respectively. Ser-158, which is phosphorylated by PKC, is located in the SH2 domain (Soula et al., 1993, J. Biol. Chem. 268:27420-27427).

FIG. 23G is an image depicting a Western blot demonstrating that Lck is phosphorylated at Ser 158 in M. leprae-infected Schwann cells, and that the phosphorylation is mediated by PKC. Immunoblots of the lysates of control and infected Schwann cells, in the absence and presence of pan-PKC inhibitor PKCI), as depicted in FIG. 23E, were reacted with phospho-specific antibody to Lck/Ser-158. Note the phosphorylation of Ser-158 of the Lck at 56-kD, but not at approximately 65-kD, which phosphorylation is abrogated by PKCI.

FIG. 23H is an image depicting a Western blot demonstrating that heregulin-β1 activates Erk1/2 but fails to phosphorylate Lck at Ser-158. Serum starved human Schwann cells were exposed to exogenous heregulin-β1 for 30 minutes and the immunoblots were reacted with phospho-specific antibodies to Lck/Ser-158 and Erk1/2.

FIG. 23I is an image depicting a gel-based phosphorylation assay demonstrating that activated Lck/Ser-158, but not PKCα/βII, directly phosphorylates Erk2 in infected human Schwann cells. In vitro kinase assays of phospho-Lck(Ser-158) (left side of image) and phospho-PKCα/βII (right side of image) using recombinant Erk2 as a substrate are depicted. Lysates of control and M. leprae-infected Schwann cells were immunoprecipitated with either phospho-LckSer 158 antibody or phospho-PKCα/βII antibody or rabbit IgG and precipitates were incubated with recombinant Erk2 in kinase buffer and then blotted with phospho-Erk1/2 antibody. The bottom left-hand panel shows the detection of phosphorylation of recombinant Erk2 by Lck/Ser158 precipitated from Schwann cells using anti-phospho-Tyr antibody, the right-most lane (designated pTyr on top and rErk2 along the bottom) depicts the same amount of recombinant Erk2 as loaded in the two left lanes incubated in kinase buffer without cell lysates and blotted with anti-phospho-tyrosine antibody.

FIG. 24, comprising FIGS. 24A through 24E, demonstrate that Lck regulates M. leprae-induced nuclear accumulation of cyclin D1 and S phase cells. FIG. 24A is an image of a Western blot depicting control and 30 day-infected human primary Schwann cells were serum starved for 48 hours in the continuous presence of Pi3-kinase- and MEK-inhibitors with or without Lck inhibitor and immunoblots of the nuclear extracts were labeled with antibody to Cyclin D1. Coomassie Blue staining is shown in the bottom panel of the image to indicate that an equal amount of proteins was loaded onto each lane of the same blot. Note the significant blockade of nuclear cyclin D1 by Lck-inhibitor.

FIG. 24B is a graph depicting cell-cycle FACS showing mean percentages of net M. leprae-induced S-phase cells (subtracted from control Schwann cells) in the absence and presence of different combination of inhibitors. Data are presented from three independent experiments (*: p<0.001).

FIG. 24C is a graph depicting that S-phase Schwann cell population induced by exogenous heregulin fails to respond to Lck inhibitors. Cell-cycle FACS analysis of mean percentage of net S phase Schwann cells in response to exogenous heregulin-β1 in the presence and absence of Lck-inhibitor is disclosed.

FIG. 24D is a diagram depicting, without wishing to be bound by any particular theory, the differential regulation of cyclin D1 nuclear accumulation and subsequent G1/S phase transition by Erk1/2 via MEK- and Lck/Ser-158-dependent pathways in response to extracellular cues. Extracellular mitogen (e.g., heregulin)-induced phosphorylation of Erk1/2 is mediated by MEK-dependent pathway and does not involve Lck/Ser-158 pathway (shaded area).

FIG. 24E is a diagram depicting, without wishing to be bound by any particular theory, the differential regulation of cyclin D1 nuclear accumulation and subsequent G1/S phase transition by Erk1/2 via MEK- and Lck/Ser-158-dependent pathways in response to intracellular cues. Intracellular M. leprae can activate Erk1/2 directly by Lck/Ser-158 pathway in the complete absence of MEK-dependent pathway (shaded area).

FIG. 25, comprising FIGS. 25A through 25E, demonstrating intracellular M. leprae modulate the expression of key cell cycle regulatory proteins.

FIG. 25A is an image depicting an immunoblot demonstrating protein levels of indicated cell cycle proteins in 30 day-cultures of human primary Schwann cells with (ML/30 days) and without (control/30 days) M. leprae. Western blots of total Schwann cell lysates were reacted with specific antibodies to indicated cell cycle proteins.

FIG. 25B is a graph depicting densitometric analysis of the expression of cell cycle proteins. The data are presented from two independent experiments (after normalization with 13-actin) using Schwann cells from two different donors (*: p<0.005).

FIG. 25C is an image of a Western blot depicting assembly of cyclin D1 with CDK4 and p21 in 30 day-infected Schwann cells. Total protein lysates of infected and non-infected Schwann cells were immunoprecipitated with cyclin D1 antibody and blotted with antibodies to CDK4, p21 and cyclin D1.

FIG. 25D is an image depicting a blot demonstrating increased E2F-DNA binding activity in M. leprae-infected Schwann cells. FIG. 25D depicts a representative example of transcription factor array showing increased binding of E2F, but not p53, from the nuclear extracts of 30 day-infected Schwann cells as compared to uninfected cells. Positive and negative controls of the arrays are depicted as (+) and (−) respectively. The bottom panel depicts a graph demonstrating the quantification of E2F-binding activity; the data from two independent experiments were normalized to internal DNA controls (p<0.005).

FIG. 25E is an image of a gel depicting a gel shift assay showing the increased DNA binding activity of E2F-1 from the nuclear extracts of 30 day-M. leprae infected Schwann cells (ML/30 days) as compared to controls. The specificity was verified by incubating 10-fold excess unlabelled E2F-1 oligonucleotide (cold) that inhibited the specific E2F-1 binding.

FIG. 26, comprising FIGS. 26A through 26C, depict M. leprae infected and non-infected Schwann cells that have grown for 30 days were transiently transfected with wild type cyclin D1 or a cyclin D1 dominant negative mutant T156A with N-terminal FLAG.

FIG. 26A is an image depicting a Western blot of total protein lysates blotted with anti-FLAG mAb to assess the expression efficiency. Note the identical protein expression pattern in both infected and non-infected Schwann cells after transfection.

FIG. 26B is an image of a representative photomicrograph of M. leprae infected Schwann cells co-transfected with 13-Gal/T156A showing a transfection efficiency of about 60% as assessed by 13-Gal staining (blue/green).

FIG. 26C is an image of a photomicrograph depicting anti-FLAG antibody labeling of M. leprae infected Schwann cells transfected with T156A mutant showing mutant cyclin D1 expression in the cytoplasm.

### DETAILED DESCRIPTION OF THE INVENTION

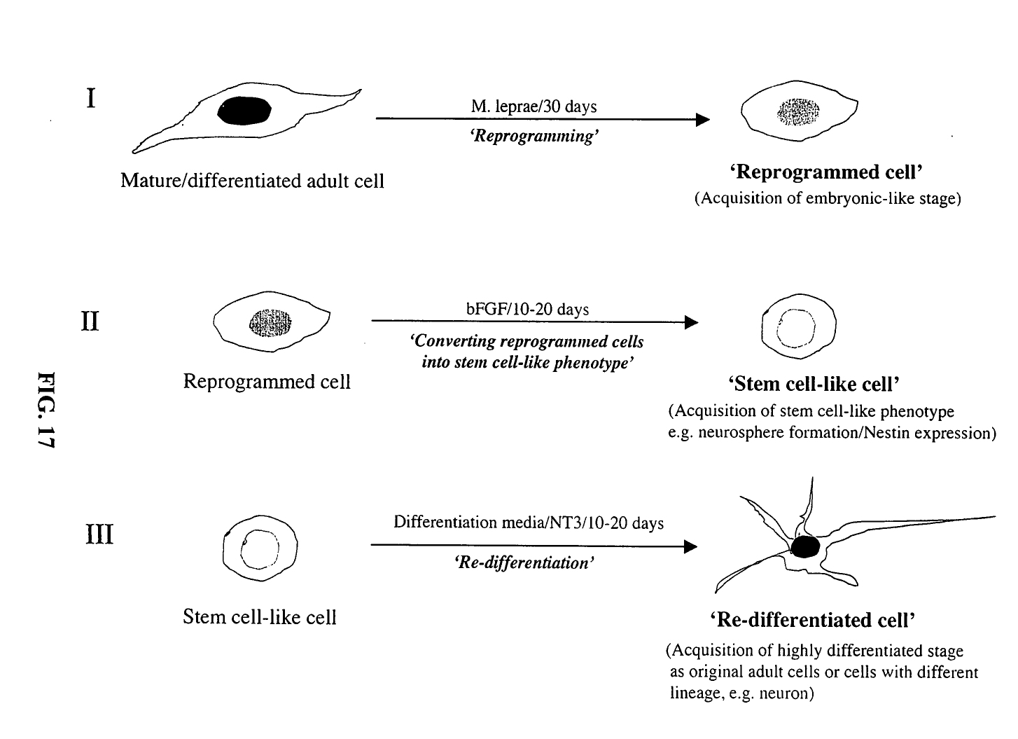

The present invention comprises a method for reprogramming of differentiated adult tissue cell into highly immature ES-like cells. That is, the invention encompasses reprogramming adult cell and then conversion of the “reprogrammed cell” to phenotypically distinct “stem cell-like cell” and re-differentiating “stem cell-like cells” to terminally differentiated cells (see, e.g., FIG. 17).

Thus the invention relates to reprogramming, converting, and re- or trans-differentiating, and thereby reprogramming a wide variety of adult differentiated cell types so that they become stem cell-like cells that can be used to produce a wide plethora of differentiated cell types.

The present invention comprises methods for reprogramming an adult cell by invasion of the cell by M. leprae, or a component thereof, and culturing the cell in the presence of the bacterium, or its component. The present invention further comprises a method of re-differentiating a cell reprogrammed according to the above-mentioned method. Such methods include production an ES-like cell, wherein the cell is cultured under conditions that facilitate the reprogramming, conversion to ES-like cell and re- or trans-differentiation of a ES-like cell into a mature cell of the same cell type/lineage or different cell type/lineage other than the cell type that it originally was. Thus, the present invention comprises a method for reprogramming a adult cell and converting a reprogrammed cell into an ES-like cell and then re-differentiating the same cell type (or same lineage) or trans-differentiating the cell into a cell type different than the original cell type (different lineage). The invention also encompasses reprogramming a cell and then re-differentiating the cell into the same type of cell as the cell was originally. These methods provide an important source of cell for use in cell-based therapies where large numbers of a desired cell can be readily produced, and uncovering the mechanisms of genetic diseases, and also in generating sources of normal and impaired tissues for use in drug discovery.

The invention further encompasses culturing and expanding the cell number to produce a population of ES-like cells for use in various therapies and the like. As demonstrated by the data disclosed herein, a cell from adult tissue can be reprogrammed to embryonic-like stage, converted to a stem cell-like cell, and re- or trans-differentiating the cell to produce various types of adult tissues according to the methods of the present invention. The reprogrammed cell exhibits, among other things; induction of a novel intracellular signaling pathway (NSP), continuous cell proliferation without detectable neoplastic transformation, expression of developmental and embryonic genes not previously expressed at a detectable level in the mature adult cell, loss of differentiated cell markers and glial cell lineage-associated genes, metabolic induction and ATP synthesis.

That is, the data herein demonstrate for the first time, that otherwise terminally differentiated adult cell cells can be reprogrammed to exhibit detectable embryonic stage phenotypes with signature ES cell genes and these reprogrammed cells can be directly converted to an ES-like cell characteristic ES-cell phenotypes. Further, the data demonstrate that the ES-like cell can then be re- or trans-differentiated into the same cell type or a cell type of the same lineage. Further, a differentiated or terminally differentiated cell can be reprogrammed into another cell type exhibiting the morphological and phenotypic characteristics of a variety of differentiated cells of a different lineage. The reprogrammed cell, after conversion to ES-like cell with stem cell phenotype(s), can then be transplanted, grafted, or infused to treat a wide plethora of diseases. Further, the present invention comprises compositions and methods for producing reprogrammed cells (e.g., ES-like cells) and their subsequent conversion to stem cell-like cells, followed by redifferentiation and/or transdifferentiation of stem cell-like cells, which cells are potentially useful in the treatment of many diseases where cell-based therapy or transplantation would provide a benefit.

The invention disclosed herein further comprises a method for the identification of proteins, lipids, glycoproteins, carbohydrates, saccharides, genes, and other components of M. leprae that are useful for, among other things, identifying functional moieties, such as, but not limited to, signaling molecules/pathways and transcriptional factors that are useful in reprogramming cells and generating ES-like cells by reprogramming adult cells as disclosed elsewhere herein. Such components can be derived from M. leprae and other organisms, including other Mycobacteria and other intracellular microorganisms, and the components with reprogramming capacity that can be identified according to the methods described herein.

The present invention further comprises novel compositions and methods for modulating the cell-division cycle of a mammalian/human cell, specifically, for regulating the cell cycle to facilitate cellular proliferation without oncogenic transformation. That is, the data disclosed herein demonstrate compositions and methods to regulate the mammalian cell cycle via a novel pathway, wherein cells, especially ES-like cells, can be induced to proliferate indefinitely, without displaying classical cancer phenotypes.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

“Cell wall” is used herein to refer to the portion of an M. leprae bacterium outside of the cell membrane, separating the cell membrane from the environment. Thus, a “whole cell wall” comprises all or at least a substantial portion of the cell wall of an M. leprae organism. A “fragment” or “fraction” of a cell wall (“cell wall fraction”) comprises a smaller portion of a cell wall compared with the intact cell wall of an untreated organism. The fragment can be isolated using size fractionation, density fractionization, charge fractionization, and the like, as would be understood in the art.

The term “component” as used herein, refers to any portion of a cell or organism that is less than the whole organism. For example, a component of a cell can comprise, but is not limited to, a cell wall, a nucleic acid, a protein, an organelle, a lipid, a sugar, a carbohydrate, a glycolipid, a glycosylated sugar, and the like, alone or in combination with another component.

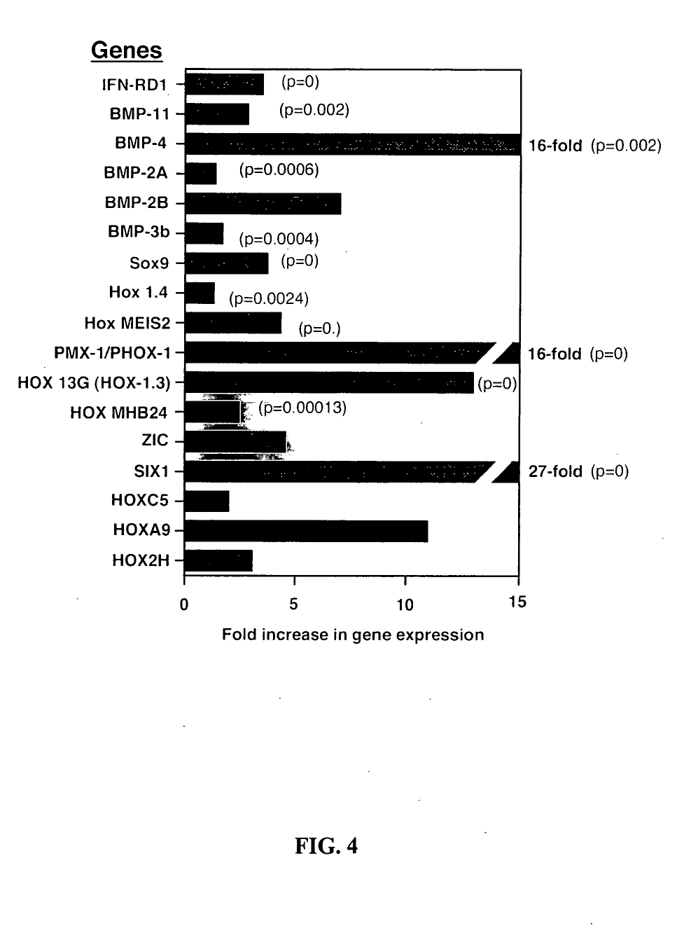

By the term “reprogramming”, as used herein, is meant conversion of a differentiated adult cell to a highly immature/embryonic-like stage cell by non-nuclear transfer or cell fusion-based methods for reprogramming. Reprogramming refers to a process for affecting a characteristic or trait of a differentiated mature cell such that the cell demonstrates a detectable increase in a trait or characteristic associated with an undifferentiated cell state compared to that same trait or characteristic in that cell prior to being subjected to the process, or in an otherwise identical but untreated cell. A trait or characteristic of an undifferentiated (embryonic-like stage) cell includes, but is not limited to, the ability to proliferate without exhibiting any detectable transformation (e.g., loss of contact inhibition, and the like), expression of proliferative markers and progenitor like genes/proteins, increased expression of a marker associated with an undifferentiated state (e.g., progenitor like genes/proteins such as those listed in, e.g., FIG. 4), and the ability to differentiate into at least one type of mature differentiated cell.

Reprogramming further preferably, but not necessarily, refers to a concomitant detectable decrease in a trait or characteristic associated with a differentiated mature cell. Such trait/characteristic can include, but need not be limited to, a lack of proliferation, expression of a marker associated with a differentiated state. Thus, “reprogrammed” refers to any increase in a trait associated with an undifferentiated state, a decrease in a trait associated with a differentiated state, or both.

A “reprogrammed cell” is an adult cell after reprogramming to an embryonic-like stage by the non-nuclear transfer or non-cell-fusion-based methods reprogramming methods disclosed herein.

The term “stem cell-like cell” as used herein, refers to a cell that was an adult/mature differentiated cell, which was reprogrammed into an ES-like cell using the methods of the invention, then converted via conversion as disclosed herein into a “stem cell-like cell” having at least one characteristic similar of a stem cell, including, but not limited to, continuous proliferation, self-renewal, formation of a neurosphere, expression of nestin, and expression of signature stem cell genes (Tables 1 and 2), and the like.

“Conversion,” as used herein, refers to conversion of a reprogrammed (embryonic-like stage) cell to a stem cell-like cell exhibiting phenotypic characteristics similar to that of stem cells.

Conversion is performed by incubating a reprogrammed ES-like cell in a “progenitor cell medium,” as the term is used herein. That is, a progenitor cell medium means medium comprised of bFGF and chicken embryo extract without added serum. Only stem cells/progenitor cells survive under these conditions whereas bFGF provides essential signals for proliferation although some differentiation may take place during long term culture.

By the term “re-differentiation” is meant differentiation of a stem cell-like cell to a mature terminally differentiated cell of the same lineage (e.g., Glial cell to neuron).

“Trans-differentiation” refers to differentiation of a stem cell-like cell to a mature terminally differentiated cell of different lineage that the cell that was initially reprogrammed and then converted in the stem cell-like cell prior to trans-differentiation. For example, such trans-differentiation includes, but is not limited to, e.g., Glial cell to a bone or a blood cell.

“Differentiated” is used herein to refer to a cell that has achieved a terminal state of maturation such that the cell has developed fully and demonstrates biological specialization and/or adaptation to a specific environment and/or function. Typically, a differentiated cell is characterized by expression of genes that encode differentiated associated proteins in a given cell. For example expression of myelin proteins and formation of myelin sheath in glial cell is a typical example of terminally differentiated glial cell.

“Differentiation medium” is used herein to refer to a cell growth medium comprising an additive such that a stem cell, embryonic stem cell, ES-like cell, neurosphere, or other such progenitor cell, that is not fully differentiated when incubated in the medium, develops into a cell with some or all of the characteristics of a differentiated cell.

An “embryonic-stage marker protein” or “gene” includes, but is not limited to, those disclosed in FIG. 4, as well as Tables 3 and 4. Such genes and markers include, but are not limited to, HOX and BMP, among many others known in the art or to be identified in the future.

“ES-like cell” or “embryonic stem-like cell” is used herein to refer to a differentiated cell that has been reprogrammed by non-nuclear transfer or cell fusion-based methods for reprogramming, to exhibit a property of an embryonic stem cell, including, but not limited to, proliferation without transformation, continuous proliferation, self-renewal and capacity to generate a wide range of tissues, the ability to differentiate into either the same or a different cell type than the original differentiated cell, and the like, compared with these same parameters in the cell prior to being de-differentiated according to the methods disclosed herein. That is, the reprogrammed cell, which was previously a mature/differentiated adult cell, has acquired an embryonic-like stage.

Basically, “stem cell” can refer to stem cells isolated from adult tissues, like brain which are not reprogrammed cells and in which case the cells can be referred to as “adult neural stem cells”. Reprogrammed cells do not form neurospheres, a characteristic property of ES cells in culture, unless they are treated with progenitor media such that the conversion step is important at this point. There is a morphological difference between and ES-like cell and a stem cell-like cell, which is likely to facilitate high growth rate as the cells float and form aggregates. Again, reprogrammed ES-like cells are attached and are not floating like the converted stem cell-like cells.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

“Neurosphere” is used herein to refer to a neural stem cell/progenitor cell wherein nestin expression can be detected, including, inter alia, by immunostaining to detect nestin protein in the cell. Neurospheres are aggregates of proliferating stem cells and the formation of neurosphere is a characteristic feature of stem cells in in vitro culture. Neurospheres normally do not form in vivo, but upon transplantation they differentiate to new tissues.

“Neural cell” is used herein to refer to a cell that exhibits morphology, function, and phenotypic characteristics similar to that of glial cells and neurons derived from the central nervous system and/or the peripheral nervous system.

“Neuron-like cell” is used herein to refer to a cell that exhibits morphology similar to that of a neuron and detectably expresses a the neuron-specific markers, such as, but not limited to, MAP2, neurofilament 200 kDa, neurofilament-L, neurofilament-M, synaptophysin, β-tubulin III (TUJ1), Tau, NeuN, a neurofilament protein, and a synaptic protein.

“Oligodendrocyte-like cell” is used herein to refer to a cell that exhibits a phenotypes similar to that of an oligodendrocyte and which expresses the oligodendrocyte-specific marker, such as, but not limited to, 0-4.

“Progression of or through the cell cycle” is used herein to refer to the process by which a cell prepares for mitosis and/or meiosis. Progression through the cell cycle includes progression through the G1 phase, the S phase, the G2 phase, and the M-phase.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of ³H-thymidine, and the like.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

“Viable M. leprae” is used herein to refer to M. leprae bacteria that are capable of growth and replication inside a cell or growth media. “Irradiated M. leprae” is used herein to refer to M. leprae bacteria that are not capable of detectable replication in a cell or growth medium after being subjected to radiation. “Non-viable” M. leprae refers to a bacterium that is not capable of detectable replication by any method known to typically enable an M. leprae bacterium to replicate.

Description

The present invention encompasses novel non-nuclear transfer methods for reprogramming a cell comprising expressing a gene isolated from M. leprae in a differentiated cell. That is, using the methods disclosed elsewhere herein, the skilled artisan can readily identify a nucleic acid encoding an M. leprae protein that when expressed in a differentiated cell, causes the cell to be reprogrammed into an ES-like cell. As the skilled artisan would appreciate based upon the disclosure provided herein, such cells are useful for, inter alia, regenerative medicine, such as treatments for Alzheimer's disease, Parkinson's disease, diabetes, heart disease, spinal cord injury, multiple sclerosis, and other diseases amenable to the introduction of healthy replacement tissues. This is because, as demonstrated by the data disclosed herein, reprogramming cells using viable or irradiated M. leprae, or a component thereof, including a nucleic acid encoding an M. leprae component, results in a differentiated cell reverting to a ES-like cell type that exhibits a pluripotent dedifferentiated cell phenotype.

The reprogrammed cell can then be re-differentiated into the same or trans-differentiated into a different cell type than the original cell type that was reprogrammed. More specifically, the reprogrammed ES-like cell is converted, according to the methods of the present invention, into a stem cell-like cell. The stem cell-like cell can then be re- or trans-differentiated into a cell type of choice. Additionally, the ES-like cell can be grown-up to produce large numbers of ES-like cells before the cells are re-differentiated to produce a cell type of interest.

Therefore, the skilled artisan would appreciate, armed with the teachings provided herein, that the present invention provides methods for producing cells of a differentiated type which cannot be readily obtained in large numbers, by producing an ES-like cell by reprogramming a mature/differentiated adult cell and then expanding the reprogrammed cell to produce a number of reprogrammed ES-like cells and even to produce ES cell-like cell lines. Also, the reprogrammed cell can be converted into a stem cell-like cell and the stem cell-like cell can be expanded to produce a plurality of such cells. Either way, the methods disclosed herein provide novel means for producing large numbers of cells which can be expanded then re-differentiated into a wide plethora of cell types as desired. The ES-like cells, stem cell-like cells, re-differentiated cells, trans-differentiated cells, or any combination thereof, can then be used for a number of cell-based therapies where such cells can provide a benefit when administered to an animal.

I. Methods of Reprogramming a Cell

The present invention comprises reprogramming a differentiated cell by contacting/invasion of the cell with an intracellular microorganism, as exemplified by M. leprae, or with a component of the bacterium. Thus, the present invention is not limited to viable or irradiated M. leprae or components thereof. Other obligate intracellular organisms display similar properties in that they are capable of modifying, altering, or otherwise changing the intracellular environment of the host cell in order to simplify the environment to facilitate their enduring survival and propagation. Such organisms include, but are not limited to, the genus Listeria, Rickettsia, Chlamydia, other species of Mycobacteria, including M. tuberculosis, M. smegmatis, M. bovis, M. kansasii, M. avium, and the like. Thus the present invention includes the use of a viable, non-viable intracellular microorganism, or components of, including, but not limited to, Listeria (Hether et al., 1983, Infect. Immun. 39: 1114-1121), Rickettsia (Smith et al., 1979, J. Bacteriol. 137: 963-971), Chlamydia (Caldwell et al., 1981, Infect. Immun. 31: 1161-1176), and Mycobacteria (Besra, 1998, Methods. Mol. Biol. 101: 91-107) for de-differentiating differentiated cells into ES-like cells. The skilled artisan would appreciate, based upon the disclosure provided herein that any non-toxic/non-cytopathic obligate intracellular bacteria and their components with no toxicity or apoptotic effect can be used in the methods disclosed herein. This is because there are common themes and common survival strategies that many intracellular pathogens share with using highly conserved microbial proteins, and thus they can be used to reprogram cells in a similar manner to M. leprae as exemplified herein. Without wishing to be bound by any particular theory, these highly conserved bacterial proteins should not induce apoptosis. Methods for infecting cells with such microorganisms are well known in the art, and when coupled with the methods of the present invention, the skilled artisan can readily reprogram a differentiated cell, identify an organism, or a component thereof, that reprograms differentiated cells, or both.

The skilled artisan, when armed with the present disclosure, will understand that M. leprae and its components comprise the whole viable organism, as well as the whole non-viable organism, including M. leprae killed or otherwise inactivated by heat sterilization, irradiation, chemical killing, such as with formaldehyde or another fixative, or with a bactericidal agent. As an example, M. leprae can be inactivated by irradiation by exposing the bacteria to 2 million rads of irradiation from a ¹³⁷Cs source, as described elsewhere herein.

However, the present invention is not limited to any particular method of inactivating or providing non-viable M. leprae. Rather, the present invention includes any method of obtaining a non-viable mycobacterium where the organism is not capable of replicating but is still physically intact, as assessed using, among other things, light and electron microscopy, or any other method of determining that an organism is physically intact as would be understood by one in the art armed with the teachings provided herein.

Further, as described elsewhere herein, a component of M. leprae includes, but is not limited to, a cell wall fraction, either crude or purified, a cell wall component, such as, but not limited to, PGL-1, and cell wall proteins. The isolation and preparation of PGL-1 is described in Hunter and Brennan, 1981, J. Bacteriol. 147: 728-735; Hunter et al., 1982, J. Biol. Chem. 257: 15072-15080; Ng et al., 2000, Cell 103: 511-524. Additionally, M. leprae cell wall fractions, either whole cell wall fraction or purified cell wall-proteins and lipid fractions, can be prepared according to methods well known in the art such as those described in, for example, Hunter and Brennan (1981, J. Bacteriol. 147: 728-735).

Components of M. leprae further comprise a secreted and non-secreted protein, a glycoprotein, a lipid, a glycolipid, a carbohydrate, and a nucleic acid comprising a portion of the genome of M. leprae, all of which are known and available publicly (Cole et al., 2001, Nature 409: 1007-1011), and which are readily identifiable using the methods disclosed elsewhere herein.

Viable or irradiated M. leprae, or a component thereof, is incubated with a differentiated cell to allow interaction of the bacterium or bacterial component with the differentiated cell. As an example, a live or irradiated whole M. leprae is incubated with a differentiated cell. The relative concentrations include, but are not limited to a concentration of about 1×10⁸ bacteria per milliliter at a cell to bacteria ratio of about 1:40. In the case where bacterial cell wall fractions are used, about 5 to about 50 micrograms per milliliter of cell wall preparation is incubated with a differentiated cell. When PGL-1 is used, from about 10 to about 50 micrograms per milliliter is incubated with the differentiated cell.

The irradiated or viable M. leprae, or component thereof, is incubated with a differentiated cell for a period of time from about 7 to about 60 days, more preferably from about 10 to about 44 days, even more preferably from about 15 to about 35 days, yet more preferably from about 15 to about 30 days. More preferably, from about 15 to 30 days. Preferably, the irradiated or viable M. leprae, or components thereof, are incubated with a cell from at least 15 days. This is because, as demonstrated by the data disclosed herein, viable or irradiated M. leprae, or components thereof, can upregulate, among other things, transcriptional factors, embryonic cell markers, and the like, and down-regulate, inter alia, differentiated cell markers and the genes that govern the differentiated phenotype.

Further, that data disclosed herein demonstrate that when a cell is contacted with M. leprae, or a component thereof, anti-apoptotic, cell cycle, growth factor, transcription factor, signaling, and metabolism genes are upregulated, whereas “differentiation-associated genes” (e.g., myelin genes and genes that influence myelin synthesis, such as ErbB3/neuregulin receptor, which is turned off by M. leprae, and the like) are down regulated. Moreover, the data disclosed herein demonstrate that M. leprae can reprogram cells into an ES-like cell state after the period of time specified above. Therefore, when a differentiated cell is contacted with M. leprae, or components thereof, the cell becomes a more primitive and pliable ES-like cell state.

Such ES-like cells are useful in for a wide variety of uses including, but not limited to, regenerative medicine, transplantation, and the treatment of diseases such as: diabetes, Alzheimer's disease, multiple sclerosis, skin grafts, melanomas, spinal cord injuries, nerve damage, Parkinson's disease, and other such diseases and injuries where the replacement of a cell or tissue with ES or ES-like cells can result in a treatment or alleviation of the disease. Further, the skilled artisan will appreciate, based upon the disclosure provided herein, that the ES-like cells of the present invention are not transformed cells, and do not exhibit any of the characteristics of an oncogenic cell (contact inhibition, actin and vinculin fiber disruption, lack of focal contacts, and the absence of tumor-suppressor transcripts and the like). Thus, the ES-like cells of the present invention can be used safely in, among other things, regenerative medicine therapies.

Preferably, the mature adult differentiated cells employed in the present invention include, but are not limited to: neural cells such as those derived from the PNS and central nervous system as a starting material (CNS) including, but not limited to, glial cells, such as, e.g., Schwann cells, astrocytes, oligodendrocytes, microglial cells, and skin cells, including epidermal cells, such as, keratinocytes, and melanocytes, and, epithelial cells, and the like, blood cells, such as lymphocytes, including T cells and B cells, macrophages, monocytes, dendritic cells, Lagerhans cells, eosinophils, and the like, adipocytes, cardiac muscle cells, fibroblasts, osteoclasts, osteoblasts, endocrine cells, β-islet cells of the pancreas, endothelial cells, epithelial cells, granulocytes, hair cells, mast cells, myoblasts, Sertoli cells, striated muscle cells, zymogenic cells, oxynitic cells, brush-border cells, goblet cells, hepatocytes, Kupffer cells, stratified squamous cells, pneumocytes, parietal cells, podocytes, synovial cells, serosal cells, pericytes, chondrocytes, osteocytes, Purkinje fiber cells, myoepithelial cells, megakaryocytes, and the like.

The present invention further includes reprogramming primary adult cells established in culture, or to be established in culture in the future, as well as using existing cell lines. Primary cell lines include, but are not limited to, keratinocytes, hepatocytes, heart muscle cells, kidney cells, and the like, and other primary and established cell lines known in the art and available from a variety of sources, including the American Type Culture Collection (Manassas, Va.).

The invention encompasses converting a reprogrammed cell into a stem cell-like cell, such that the reprogrammed cell acquires a stem cell-like phenotype. Such stem cell-like phenotype includes, but is not limited to, the ability to form neurospheres and the expression of nestin protein as well as signature stem cell genes. The skilled artisan would appreciate, once armed with the teachings provided herein, that the reprogrammed cell can be converted into a stem cell-like cell, which converted cell can acquire a vast plethora of stem cell-like phenotypes as are well-known in the art.

The present invention encompasses methods to re-differentiate the stem cell-like cells after reprogramming an adult cell enumerated herein and a wide plethora of cells known in the art or identified in the future, and conversion of the reprogrammed cell into the stem cell-like cell. This is because, as disclosed elsewhere herein, M. leprae binds α-dystroglycan via laminin-2, both of which are present on most cell membranes, and therefore the present invention is applicable to any cell comprising α-dystroglycan, which is expressed in most of cells. As an example, the dystroglycan dimer, consisting of the transmembrane β-subunit and the extracellular α-subunit, is present in kidney, liver, stomach, uterus, skeletal muscle, salivary glands, small intestine, smooth muscle, villi epithelia, pancreas, trachea, mammary gland, testis, skin, intestinal epithelia, and hepatocytes (Durbeej et al., 1998, J. Hist. Cyto. 46: 449-457). Further, because dystroglycan links extracellular laminin-2 to the intracellular cytoskeleton, laminin-2 is also present on a wide plethora of cell types. Thus, because M. leprae and its components bind α-dystroglycan via laminin-2, the methods of the present invention are amenable to virtually any cell type, including those listed herein and others well known in the art. Further, as demonstrated by the data disclosed herein, human cells are capable of growing at temperatures permissive for M. leprae growth without any detectable functional, morphological, or phenotypic alterations. Accordingly, due to the universal presence of α-dystroglycan and laminin-2 on many mammalian cell types and due to the adaptability of human cells and M. leprae, the present invention can be used to manipulate the differentiation status of virtually any mammalian cell.

Methods for obtaining primary cultures of the aforementioned cells and others are well known in the art, and usually include obtaining the tissue from a biopsy, amputated limb, secretion, excretion, or other source. The sample comprising the desired cells is then minced or otherwise cut into smaller pieces or treated to release the cell. For instance, and without limiting the invention to any particular method of obtaining a cell to be used in the methods described herein, the tissue is often treated with a collagenase or other protease in order to disassociate the cells from the tissue aggregate. These cells are then placed in a tissue culture flask, or dish, along with a nutrient tissue culture media and propagated at a suitable temperature. In the case of human cells, that temperature is often about from about 35° C. to about 37° C.

Blood cells and lymphocytes are often obtained from whole blood treated with heparin or another anti-coagulant. The blood is centrifuged on a gradient, such as a Ficoll gradient, and the lymphocytes and other blood cells form a distinct layer often referred to as the “buffy coat”. Primary lymphocytes procured by this method can be further separated by their adherence to glass or plastic (monocytes and macrophages adhere, other lymphocytes, in general, do not adhere). Methods for obtaining and culturing both solid tissue and blood cells from a human are well known in the art and are described in, for example, Freshney (2000, Culture of Animal Cells: A Manual of Basic Techniques, 4th Edition, Wiley-Liss, New York, N.Y.).

The skilled artisan would appreciate, based upon the disclosure provided herein, that the particular method for obtaining a cell of interest is not limited in any way. That is, any method of obtaining a cell to be trans-differentiated, re-differentiated, or both, according to the methods disclosed herein, is not limited in any way, but encompasses methods for isolating a cell of interest well known in the art or to be developed in the future. As a non-limiting example, in order to isolate Schwann cells, nerve tissue comprising Schwann cells (e.g., peripheral nerve tissue) is derived from a mammal, preferably a human. The tissue is derived from an adult patient, since this facilitates autologous transplants and thus reduces the likelihood of adverse immunogenic reactions in the patient.

Peripheral nerve tissue can be obtained using surgical procedures such as nerve biopsies, amputated limbs, and from organ donors and by any other methods well known in the art or to be developed. Potential sources of peripheral nerve include the sciatic nerve, cauda equina, sural nerve of the ankle, the saphenous nerve, the sciatic nerve, or the brachial or antebrachial nerve of the upper limb.

A preferred amount for the starting nerve tissue is between about 10 milligrams to about 10 grams, preferably between about 100 milligrams to about 1-2 gram. Primary human Schwann cells can be isolated and cultured using the methods detailed elsewhere in this invention or methods known in the art. Other methods for the isolation and culture of Schwann cells and other neural cells are well known in the art, and can readily be employed by the skilled artisan, including methods to be developed in the future. The present invention is in no way limited to these or any other methods, of obtaining a cell of interest.

As a further non-limiting example, normal keratinocytes can be isolated from skin tissues well-known in the art, including newborn human foreskin, skin biopsies, and the like. Cultures are established by plating aliquots of single cell suspensions in the presence of mitomycin C-treated Swiss mouse 3T3 fibroblasts as described in, for example, Allen-Hoffmann, et al. (1984, Proc. Nat'l. Acad. Sci. USA 81: 7802-7806). Standard keratinocyte culture medium usually comprises a mixture of Ham's F12:Dulbecco's modified Eagle's medium (DME), (3:1, 0.66 mM calcium) supplemented with 2.5% fetal calf serum (FCS), 0.4 micrograms/milliliter hydrocortisone, 8.4 nanograms/milliliter cholera toxin, 5 micrograms/milliliter insulin, 24 micrograms/milliliter adenine, 10 nanograms/milliliter epidermal growth factor, 100 units penicillin and 100 micrograms/milliliter streptomycin. Cells are passaged at about weekly intervals at about 3×10⁵ cells on a 100 mm² tissue culture dish with feeder cells.

Alternative methods for isolating and culturing primary human keratinocytes are well known in the art, and are described in, for example, U.S. Pat. No. 5,795,781, and in Rheinwald and Green (1975, Cell 6: 331-43).

The present invention further comprises methods for reprogramming cells using a component of M. leprae such as, e.g., PGL-1, a cell wall, a portion of a cell wall, and the like. That is, the present invention comprises contacting a component of M. leprae with a cell in order to reprogram the cell into an ES-like cell state. The component can comprise an M. leprae gene, an M. leprae protein, a cell wall component, such as PGL-1, a saccharide or variant thereof, a glycolipid, glycoprotein, lypoarabinomanen (LAM) or other components detailed elsewhere herein.

Methods of identifying the component that mediates the reprogramming would be well-understood by the skilled artisan, based on the disclosure provided herein, such as those set forth elsewhere herein.

The skilled artisan, based upon the disclosure provided herein, would appreciate that the present invention encompasses a method for producing ES-like cells from existing differentiated cells from adult tissues, which is also referred herein as “reprogramming” a cell. Briefly, the cells are isolated from an animal, or alternatively, obtained from an existing or discovered cell line, reprogrammed using the methods disclosed elsewhere herein, and then returned to the animal as a therapeutic approach to regenerative medicine.

Further, as disclosed herein, the cells can be stored in order to establish a cell library to offer cells and tissues for regenerative medicine therapies and for future research into the nature of stem cells and their molecular mechanisms. Additionally, the cells can be expanded to produce greater numbers of reprogrammed ES-like cells. The ES-like cells can be administered to an animal, or, alternatively, the expanded ES-like cells can be converted into a stem cell-like cell, which cell can then be re-differentiated into the same, or trans-differentiated into a different, cell type and the cells produced can be administered to an animal.

In this way, differentiated cells that cannot be expanded readily in cell number (e.g., because they do not proliferate and/or do not proliferate sufficiently to provide adequate numbers of cells) can be produced by simply reprogramming them according to the methods disclosed herein, then expanding the resultant ES-like and/or stem cell-like cells, and then re- and/or trans-differentiating the expanded cells thereby producing large numbers of reprogrammed cells of interest, which can be used in a wide plethora of therapeutic methods.

Further, the reprogrammed ES-like cells can be transfected or transformed with a nucleic acid encoding a protein that, when the protein is expressed in the cells, provides a beneficial effect to the animal to which the cells are administered. That is, in essence, the reprogrammed cells of the invention can be used in cell-based and gene-therapy based therapies to provide cells and/or to provide a recombinant cell expressing an exogenous nucleic acid to an animal. This method can be extremely useful for treating diseases with genetic defects, particularly genetic nerve degenerative diseases in which genetic defects of neurons cannot be reversed.

II. Method for Reprogramming a Cell

The present invention further comprises novel non-nuclear transfer or non-cell-fusion (which differ from any nuclear transfer or cell fusion-based methods of reprogramming a cell) methods for reprogramming an adult mature differentiated cell to produce an ES-like cell according to the methods disclosed herein, into any suitable cell in order to use the cells for, inter alia, transplantation, infusion, and the like, into a patient. The reprogrammed cell of the present invention can also be stored in a cell library to facilitate ready access to a wide variety of cell types for research and regenerative medicine purposes.

One skilled in the art would, based upon the disclosure provided herein, understand that the present invention encompasses reprogramming a differentiated cell according to the methods disclosed herein. The reprogrammed cell after converting to ES-like phenotype, also referred to herein as an “ES-like cell”, among other things, can then be re-differentiated, with or without having been expanded in number before being re-differentiated, to produce a terminally differentiated cell of the same type or same cell lineage as was initially reprogrammed, or to produce a terminally differentiated cell of a different type of different cell lineage than the original cell.

Methods for reprogramming a cell are more fully set forth elsewhere herein. Methods for re-differentiating ES and ES-like cells are well known in the art, and are amply demonstrated by the data disclosed herein, and all such methods, as well as methods developed in the future, are encompassed by the invention. For instance, re-differentiation of ES and ES-like cells usually comprises the addition or subtraction of one or more extracellular signals, such as, but not limited to a growth factor, in order to differentiate an ES-like or ES cell into a differentiated, specialized cell. Further, ES cells may spontaneously differentiate if grown to confluence and allowed to remain in culture (See, e.g., U.S. Pat. No. 6,200,806). Differentiation of ES cells to produce specific tissue types is well known and is described more fully elsewhere herein.

For example, in order to re-differentiate neural ES or ES-like cells into neurons, glial cells, oligodendrocytes, astrocytes, or other neural cells, ES-like cells can be plated on poly-lysine-coated tissue culture plates or slides in DMEM with 10% fetal bovine serum. Under these conditions, re-differentiation occurs spontaneously, and can be assessed using methods well known in the art. Confirmation of the differentiated phenotype can be determined using techniques well known in the art and described elsewhere herein.

In addition, proliferating ES-like neurospheres can be induced to re-differentiate by removal of the growth factor mitogens and LIF, and providing 1% serum, a substrate (e.g., glass cover slip or extracellular matrix components), a source of ionic charges (e.g., poly-ornithine) as well as a mixture of growth factors including 10 ng/ml PDGF A/B, 10 ng/ml CNTF, 10 ng/ml IGF-1, 10 μM forskolin, 30 ng/ml T3, 10 ng/ml LIF and 1 ng/ml NT-3. This differentiation protocol produces cell cultures highly enriched in neurons (i.e., greater than 35% of the differentiated cell culture) and enriched in oligodendrocytes.

Numerous other in vivo and in vitro methods of differentiating ES-like cells into specialized adult cells are well known in the art. For example, methods have been described for: differentiating ES-like cells into neural and muscle cells (U.S. Pat. No. 6,432,711), differentiating ES-like cells into adipogenic cells and tissues (U.S. Pat. No. 6,322,784), differentiating ES-like cells into retinal cells and tissues (U.S. Pat. No. 6,117,675), differentiating an ES-like cell into a glial cell (U.S. Pat. No. 6,033,906), differentiating ES-like cells into smooth muscle cells (U.S. Pat. No. 6,001,654), differentiating ES-like cells into bone, cartilage, muscle and fat cells (U.S. Pat. No. 5,827,735), and for differentiating ES-like cells into reticulocytes (U.S. Pat. No. 5,911,301).

As yet another example, ES-like cells can be differentiated into human glioblasts as follows. Proliferating ES-like neuroblasts are removed from proliferation medium and plated onto poly-ornithine plastic tissue culture dishes in a mixture of N2 media with the mitogens EGF, bFGF and LIF as described above, as well as 0.5% FBS. 0.5 milliliters of N2 medium and 1% FBS is added and the cells are incubated until morphological changes are apparent. Immunohistochemistry and gene expression analysis, described below, are used to verify the differentiation of ES-like cells into glioblasts.

As a further example, keratinocyte-derived ES-like cells can be differentiated into epidermis using the defined keratinocyte media described above. Briefly, ES-like cells are seeded at about 1000 cells per centimeter, and re-fed the proliferation media about every other day until the cells are from about 80% to about 100% confluent. As demonstrated by the data disclosed herein, very few of the ES-like cells of the present invention are in the G1 phase of the cell cycle, so they do not demonstrate parasynchronous growth arrest (Shipley et al., 1986, Cancer Res. 46:2068-2071). Within about 48 hours to about 96 hours, the ES-like cells begin to stratify into to form a multilayered epithelium, the end result being an extended sheet of multilayered epidermis. After a period of time, the granular cells of the multilayered epithelium begin to mature into cornified, anucleate cells which form the topmost layer of a complete epidermis.

Methods to assess the re-differentiated phenotype of an ES-cell type are well known in the art and described elsewhere herein. In general, monoclonal or polyclonal antibodies can be used to probe re-differentiated ES-like cells to detect the presence of specific cell markers characteristic of the re-differentiated cell type of interest. A wide variety of monoclonal and polyclonal antibodies for such markers available commercially (e.g., Santa Cruz Biotechnology, Santa Cruz, Calif.). The re-differentiated ES-like cell is then contacted with a second monoclonal or polyclonal antibody that binds to the first monoclonal or polyclonal antibody. The second antibody is usually conjugated to molecule that allows detection of the antibody, such as a molecule that emits fluorescent light at certain wavelengths (e.g., Texas Red, FITC, Rhodamine, and the like), a molecule that creates a detectable signal upon treatment with certain chemicals (horseradish peroxidase, alkaline phosphatase, etc.), a radiolabeled, or other detectable moieties (e.g., biotin, PE, and the like) all of which are well known in the art or as developed in the future.

As exemplified elsewhere herein, it can be determined if an ES-like cell has re-differentiated into a neuron using the methods disclosed elsewhere herein, including the expression of neuron-specific markers: MAP2ab and MAP2c (microtubule-associated protein in developing neurons and dendrites of mature neurons, respectively), NeuN (neuron-specific nuclear protein), neurofilament-L, neurofilament-M, neurofilament-200-kDA (axonal polypeptides, intermediate filaments), β-tubulin III/TUJ1 (neuron cytoskeletal protein recognized by TUJ1 antibody), synaptophysin (presynaptic vesicle membrane polypeptide) and Tau (microtubule associated protein and an essential component of the axon). Further, the absence of the S-100 marker can indicate that the ES-like cell no longer demonstrated a Schwann cell phenotype. As an example, in order to determine if an ES-like cell has re-differentiated into an oligodendrocyte, cells were fixed for 10 minutes at room temperature with 4% paraformaldehyde. Cells were washed about three times for 5 minutes each with 0.1 M PBS, pH 7.4. Cell preparations were blocked for about 1 hour at room temperature in 5% normal serum diluted in 0.1M PBS, pH 7.4. Cells were incubated with primary antibodies to the oligodendrocyte marker O4 (Boehringer Mannheim No. 1518 925) diluted in 1% normal serum for 2 hours at room temperature. Preparations were then washed twice for about 5 min with 0.1 M PBS. Cells were incubated with secondary antibodies, and viewed according to the manufacturer's instructions for the secondary antibody.

However, the present invention is not limited to these, or any other, particular methods for assessing the differentiated state of a cell for purposes of this invention. The skilled artisan, when armed with the present disclosure and the methods presented herein, will be able to re-differentiate an ES-like cell into virtually any type of cell and to determine if the ES-cell is expressing the characteristic markers of that cell type.

Further, the skilled artisan, when equipped with the present disclosure and the methods herein, can re-differentiate an ES-like cell into virtually any cell or tissue type using standard cell culture or in vivo techniques with appropriate growth factors/conditions. In addition, the data disclosed demonstrate that ES-like cells derived from human Schwann cells can re-differentiate into neuron-like cells in the presence of, inter alia, retinoic acid and NT3 media. Such neuron-like cells show an astounding morphological and phenotypic similarity to human neurons throughout the development process, as well as decreased expression of the Schwann cell-specific marker S-100 and highly increased expression of neuron-specific markers such as MAP2, NeuN, TUJ1, synaptic proteins and all neurofilament markers expressed in both developing and mature neurons. While the present invention is not limited to these, or any other differentiation markers, the present invention encompasses methods for generating re-differentiated cells from ES-like stem cells where markers known to be associated with a differentiated cell state become detectable in the cell where they were not detectable prior to the treatment.

In addition, the ES-like cells of the present invention can be re-differentiated in vivo. As is well known in the art, the pluripotent nature of ES-like cells permits the signals received from surrounding cells and the extracellular matrix to guide the development and differentiation of cells into virtually any cell type. Briefly, reprogrammed ES-like cells are transplanted into the specific anatomical site in which new, productive cells are necessary. As an example, in order to treat Parkinson's disease, ES-like cells, after brief stimulation with appropriate growth factors, are transplanted into the brain. In order to replace the damaged nerves in spinal cord injuries, ES-like cells are transplanted to spinal cord. In order to replace muscle tissue, ES-like cells are transplanted into muscle, preferably skeletal muscle. In order to provide bone marrow to a patient, the ES-like cells of the present invention are transplanted into the inner lumen of the bones, preferably the humerus. For skin-related applications, the ES-like cells may be transplanted by subcutaneous or intradermal injection. Methods for the preparation and transplantation of ES-like cells are extensively detailed elsewhere herein.

III. Methods Relating to Production of ES-Like Cell Library

The present invention also encompasses methods for producing a library of ES-like cells. That is, instead of re-differentiating an ES-like cell into a mature cell type, the ES-like cell can be used to create a library of ES-like cells which are useful, inter alia, for research, therapeutic, and other purposes. The skilled artisan would appreciate, based upon the disclosure provided herein, that the particular method for producing a library of interest is not limited in anyway, but encompasses methods for producing a library of interest well known in the art or to be developed in the future.

As an non-limiting example, Schwann cells, keratinocytes, and other cells enumerated elsewhere herein can be collected from a series of donors and de-differentiated to an ES-like cell state. They are then available for any future medical needs of the donor, can be used as a reservoir of ES-like cells for future use, or can be haplotyped in order to determine their immunological compatibility with other donors for future regenerative medicine needs.

The present invention is a vast improvement over prior art. Unlike such prior art, the present invention provides a readily available pool of ES-like cells that are available for immediate propagation, re-differentiation and transplantation. Further, the present invention is not restricted solely to bone marrow, but rather, as disclosed herein, to a wide and useful variety of cell and tissue types, such as neurons, astrocytes, glial cells, oligodendrocytes, keratinocytes, hepatocytes and many others.

The reservoir of ES-like cells can be constructed by obtaining and de-differentiating useful cell types as detailed above. The cells, once reprogrammed, can be assessed to confirm the ES-like phenotype and genotype, and can then be frozen for future use. Methods of freezing or cryopreserving ES-like cells are known in the art, and may comprise placing about one to about ten million cells in “freeze” medium which comprising the progenitor medium described above, absent the growth factor mitogens, about 10% BSA, and about 7.5% DMSO. The cells are then centrifuged and growth medium is aspirated and replaced with freeze medium. Cells are resuspended gently as spheres, not as dissociated cells. The ES-like cells are then slowly frozen (e.g., placing in a styrofoam container at −80° C. or an isopropanol containing freezing chamber). Cells are thawed by swirling in a 37° C. bath, resuspended in fresh progenitor medium, and grown as described above. Another media for cryopreserving ES-like cells comprises 10% DMSO, 50% FBS, and 40% bFGF-containing growth medium. The cell/media suspension is slowly brought to −140° C. for long-term storage. Cells are thawed by placing vials in a 37° C. water bath and, following gentle removal from the vial, resuspended and cultured in excess growth medium. The media is changed approximately 8 hours after thawing to clear the DMSO.

The ES-like cells of the present invention can also be immortalized to ensure a continuous supply of cells. While immortalized cells are not typically useful for transplantation or other therapeutic purposes due to the increased chance of cancer or other neoplasias, immortalized ES-like cells are a useful and valuable research tool. ES-like cells and their differentiated progeny can be immortalized or conditionally immortalized using techniques well known in the art. Conditional immortalization techniques contemplated in the present invention are, for example, Tet-conditional immortalization (see WO 96/31242, incorporated herein by reference), and Mx-1 conditional immortalization (see WO 96/02646, incorporated herein by reference).

IV. Methods of Using ES-Like Cells

The present invention also includes methods for treating a variety of diseases using an ES-like cell produced according to the novel methods disclosed elsewhere herein. The skilled artisan would appreciate, based upon the disclosure provided herein, the value and potential of regenerative medicine in treating a wide plethora of diseases including, but not limited to, heart disease, diabetes, skin diseases and skin grafts, spinal cord injuries, Parkinson's disease, multiple sclerosis, Alzheimer's disease, and the like. The present invention encompasses methods for administering ES-like cells to an animal, including humans, in order to treat diseases where the introduction of new, undamaged cells will provide some form of therapeutic relief.

ES-cells can be administered as reprogrammed cell or, following conversion, as stem cell-like cells. The skilled artisan will readily understand that ES-like cells can be administered to an animal as a re-differentiated cell, for example, a neuron, and will be useful in replacing diseased or damaged neurons in the animal. Additionally, an ES-like cell can be administered and upon receiving signals and cues from the surrounding milieu, can re-differentiate into a desired cell type dictated by the neighboring cellular milieu. Methods for re-differentiating ES-like cells in vitro are disclosed above, and the reprogrammed cells can be administered to an animal in the manner described herein. Alternatively, the cell can be re-differentiated in vitro and the differentiated cell can be administered to a mammal in need there of.

The reprogrammed ES-like cells can be prepared for grafting to ensure long term survival in the in vivo environment. For example, cells are propagated in a suitable culture medium, such as progenitor medium, for growth and maintenance of the cells and allowed to grow to confluency. The cells are loosened from the culture substrate using, for example, a buffered solution such as phosphate buffered saline (PBS) containing 0.05% trypsin supplemented with 1 mg/ml of glucose; 0.1 mg/ml of MgCl₂, 0.1 mg/ml CaCl₂ (complete PBS) plus 5% serum to inactivate trypsin. The cells can be washed with PBS using centrifugation and are then resuspended in the complete PBS without trypsin and at a selected density for injection.

In addition to PBS, any osmotically balanced solution which is physiologically compatible with the host subject may be used to suspend and inject the donor cells into the host. Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

The mammalian brain has traditionally been considered to be an immunologically privileged organ, and therefore, an immune reaction to the implanted re-differentiated or ES-like cells is typically not an issue. Further, because the methods of the present invention allows a patient's own cells to be removed, reprogrammed and, optionally, converted to stem cell-like cells, and then returned to the same patient, it is in essence, an autologous transplant that will not cause a deleterious immune reaction in the patient receiving the transplant. However, the skilled artisan will appreciate that the present invention can comprise treating a patient with an immunomodulatory compound prior to and following transplant. Such compounds are well known in the art, and include, but are not limited to cyclosporin A, prednisone, azathioprine, tacrolimus, mycophenylate motefil, OKT3, and thymoglobulin. Such drugs are well known in the transplant field, as are the dosages and indications for effective use.

The invention also encompasses grafting ES-like cells in combination with other therapeutic procedures to treat disease or trauma in the body, including the CNS, PNS, skin, liver, kidney, heart, pancreas, and the like. Thus, ES-like cells of the invention may be co-grafted with other cells, both genetically modified and non-genetically modified cells which exert beneficial effects on the patient, such as chromaffin cells from the adrenal gland, fetal brain tissue cells and placental cells. Therefore the methods disclosed wherein can be combined with other therapeutic procedures as would be understood by one skilled in the art once armed with the teachings provided herein.

The ES-like cells of this invention can be transplanted “naked” into patients using techniques known in the art such as i.e., those described in U.S. Pat. Nos. 5,082,670 and 5,618,531, each incorporated herein by reference, or into any other suitable site in the body.

The ES-like cells can be transplanted as a mixture/solution comprising of single cells or a solution comprising a suspension of a cell aggregate. Such aggregate can be approximately 10-500 micrometers in diameter, and, more preferably, about 40-50 micrometers in diameter. An ES-like cell aggregate can comprise about 5-100, more preferably, about 5-20, cells per sphere. The density of transplanted cells can range from about 10,000 to 1,000,000 cells per microliter, more preferably, from about 25,000 to 500,000 cells per microliter.

Transplantation of the ES-like cell of the present invention can be accomplished using techniques well known in the art as well as those described herein or as developed in the future. The present invention comprises a method for transplanting, grafting, infusing, or otherwise introducing ES-like cells into an animal, preferably, a human. Exemplified below are methods for transplanting ES-like cells into the brains of both rodents and humans, but the present invention is not limited to such anatomical sites or to those animals. Also, methods for bone transplants are well known in the art and are described in, for example, U.S. Pat. No. 4,678,470, pancreas cell transplants are described in U.S. Pat. No. 6,342,479, and U.S. Pat. No. 5,571,083, teaches methods for transplanting cells, such as ES-like cells, to any anatomical location in the body.

In order to transplant ES-like cell into a rodent, adult rodents are anesthetized, preferably with sodium pentobarbitol (45 mg/kg, i.p.) or a ketamine/rompun cocktail well known in the art. The animal is then positioned in a Kopf stereotaxic instrument (Tujunga, Calif.). A midline incision is made in the scalp and a hole drilled for the injection of cells. Rodents receive implants of undifferentiated human ES-like cells into the left striatum using a glass capillary attached to a 10 μl Hamilton syringe. Each animal receives a total of about 250,000-500,000 cells in a total volume of about 2 μl. Cells are transplanted about 1-2 days after passaging and the cell suspension comprises undifferentiated stem cell clusters of about 5-20 cells. Following implantation, the skin was sutured closed with either thread or staples. After recovery, animals are behaviorally tested and sacrificed for histological and immunological analysis to determine the differentiation of stem cell-like cells to neurons in vivo.

During the generation of stem cell-like cells, reprogrammed ES-like cells undergo extensive expansion, which are then further subjected to multiple divisions to generate stem cell-like cell lines. This gradually dilutes the bacterial load to zero because irradiated M. leprae do not multiply. Therefore, the final stem cell-like cells are free of bacteria. These cells, after further checking the absence of bacterial genome, are safe and suitable for direct cell therapy.

In order to transplant ES-like cells into a human, ES-like cells are prepared as described above. Preferably, the ES-like cells are from the patient they are being transplanted into, but at the minimum, blood type or haplotype compatibility should be determined. Surgery is performed using a Brown-Roberts-Wells computed tomographic (CT) stereotaxic guide. The patient is given local anesthesia in the scalp area and intravenously administered midazolam. The patient undergoes CT scanning to establish the coordinates of the region to receive the transplant. The injection cannula usually consists of a 17-gauge stainless steel outer cannula with a 19-gauge inner stylet. This is inserted into the brain to the correct coordinates, then removed and replaced with a 19-gauge infusion cannula that has been preloaded with about 30 μl of tissue suspension. The cells are slowly infused at a rate of 3 μl/min as the cannula is withdrawn. Multiple stereotactic needle passes are made throughout the area of interest, approximately 4 mm apart. The patient is examined by CT scan postoperatively for hemorrhage or edema. Neurological evaluations are performed at various post-operative intervals, as well as PET scans to determine metabolic activity of the implanted cells.

The cells may also be encapsulated and used to deliver biologically active molecules, according to known encapsulation technologies, including microencapsulation (see, e.g., U.S. Pat. Nos. 4,352,883; 4,353,888; and 5,084,350, herein incorporated by reference), or macroencapsulation (see, e.g., U.S. Pat. Nos. 5,284,761; 5,158,881; 4,976,859; and 4,968,733; and International Publication Nos. WO 92/19195; WO 95/05452, all of which are incorporated herein by reference). For macroencapsulation, cell number in the devices can be varied; preferably, each device contains between 10³-10⁹ cells, most preferably, about 10⁵ to 10⁷ cells. Several macroencapsulation devices may be implanted in the patient. Methods for the macroencapsulation and implantation of cells are well known in the art and are described in, for example, U.S. Pat. No. 6,498,018.

ES-like cells of the present invention can also be used to express a foreign protein or molecule for a therapeutic purpose or for a method of tracking their integration and differentiation in a patient's tissue. Thus, the invention encompasses expression vectors and methods for the introduction of exogenous DNA into ES-like cells with concomitant expression of the exogenous DNA in the ES-like cells such as those described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

The isolated nucleic acid can encode a molecule used to track the migration, integration, and survival of ES-like cells once they are placed in the patient, or they can be used to express a protein that is mutated, deficient, or otherwise dysfunctional in the patient. Proteins for tracking can include, but are not limited to green fluorescent protein (GFP), any of the other fluorescent proteins (e.g., enhanced green, cyan, yellow, blue and red fluorescent proteins; Clontech, Palo Alto, Calif.), or other tag proteins (e.g., LacZ, FLAF-tag, Myc, His₆, and the like) disclosed elsewhere herein. Alternatively, the isolated nucleic acid introduced into the ES-like cell can include, but are not limited to CFTR, hexosaminidase, and other gene-therapy strategies well known in the art or to be developed in the future.

Tracking the migration, differentiation and integration of an ES-like cell of the present invention is not limited to using detectable molecules expressed from a vector or virus. The migration, integration, and differentiation of a cell can be determined using a series of probes that would allow localization of transplanted ES-like cells. Such probes include those for human-specific Alu, which is an abundant transposable element present in about 1 in every 5000 base pairs, thus enabling the skilled artisan to track the progress of an ES-like cell transplant. Tracking an ES-like cell transplant may further be accomplished by using antibodies or nucleic acid probes for cell-specific markers detailed elsewhere herein, such as, but mot limited to, NeuN, MAP2, neurofilament proteins, and the like.

Expression of an isolated nucleic acid, either alone or fused to a detectable tag polypeptide, in ES-like cells can be accomplished by generating a plasmid, viral, or other type of vector comprising the desired nucleic acid operably linked to a promoter/regulatory sequence which serves to drive expression of the protein, with or without tag, in ES-like cells in which the vector is introduced. Many promoter/regulatory sequences useful for driving constitutive expression of a gene are available in the art and include, but are not limited to, for example, the cytomegalovirus immediate early promoter enhancer sequence, the SV40 early promoter, as well as the Rous sarcoma virus promoter, and the like. Moreover, inducible and tissue specific expression of the desired nucleic acid may be accomplished by placing the desired nucleic acid, with or without a tag, under the control of an inducible or tissue specific promoter/regulatory sequence. Examples of tissue specific or inducible promoter/regulatory sequences which are useful for this purpose include, but are not limited to the MMTV LTR inducible promoter, and the SV40 late enhancer/promoter. In addition, promoters which are well known in the art which are induced in response to inducing agents such as metals, glucocorticoids, hormones, antibiotics (such as tetracycline) and the like, are also contemplated in the invention. Thus, it will be appreciated that the invention includes the use of any promoter/regulatory sequence, which is either known or unknown, and which is capable of driving expression of the desired protein operably linked thereto.

Where the expression of a dysfunctional protein causes a disease, disorder, or condition associated with such expression, the expression of the corrected protein from ES-like cells driven by a promoter/regulatory sequence can provide useful therapeutics including, but not limited to, gene therapy. Diseases, disorders and conditions associated with a dysfunctional protein are disclosed elsewhere herein and are well known in the art.

Selection of any particular plasmid vector or other DNA vector is not a limiting factor in this invention and a vast plethora of vectors are well-known in the art. Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

The invention thus includes an ES-like cell comprising a vector encoding an isolated nucleic acid encoding a desired protein or other molecule. The incorporation of a desired nucleic acid into a vector and the choice of vectors is well-known in the art as described in, for example, Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

The invention also includes ES-like cells, viruses, proviruses, and the like, containing such vectors. Methods for producing cells comprising vectors and/or exogenous nucleic acids are well-known in the art. See, for example, Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York). Methods for expressing a protein from a virus infecting an ES-like cell are well known in the art, and are described in, for example, U.S. Pat. No. 6,528,306.

The nucleic acids encoding the desired protein may be cloned into various plasmid vectors. However, the present invention should not be construed to be limited to plasmids, or to any particular vector. Instead, the present invention encompasses a wide plethora of vectors which are readily available and/or well-known in the art or such as are developed in the future.

The invention also includes a recombinant ES-like cell comprising, inter alia, an isolated nucleic acid. In one aspect, the recombinant cell can be transiently transfected with a plasmid encoding a portion of a desired nucleic acid. The nucleic acid need not be integrated into the cell genome nor does it need to be expressed in the cell. Moreover, the cell may be any type of eukaryotic cell and the invention should not be construed to be limited to any particular cell line or cell type. Such cells include, but are not limited to, Schwann cells, keratinocytes, liver cells, pancreas cells, muscle cells, epithelial cells, kidney cells, heart muscle cells, skin cells, and the like.

The invention includes a eukaryotic cell which, when a transgene of the invention is introduced therein, and the protein encoded by the desired gene is expressed therefrom, where it was not previously present or expressed in the cell or where it is now expressed at a level or under circumstances different than that before the transgene was introduced, a benefit is obtained. Such a benefit may include the fact that there has been provided a system wherein the expression of the desired gene can be studied in vitro in the laboratory or in a mammal in which the cell resides, a system wherein cells comprising the introduced gene can be used as research, diagnostic and therapeutic tools, and a system wherein mammal models are generated which are useful for the development of new diagnostic and therapeutic tools for selected disease states in a mammal.

An ES-like cell expressing a desired isolated nucleic acid can be used to provide the product of the isolated nucleic acid to a cell, tissue, or whole mammal where a higher level of the gene product can be useful to treat or alleviate a disease, disorder or condition associated with abnormal expression, and/or activity. Therefore, the invention includes an ES-like cell expressing a desired isolated nucleic acid where increasing expression, protein level, and/or activity of the desired protein can be useful to treat or alleviate a disease, disorder or condition.

One of ordinary skill would appreciate, based upon the disclosure provided herein, that a “knock-in” or “knock-out” vector of the invention comprises at least two sequences homologous to two portions of the nucleic acid which is to be replaced or deleted, respectively. The two sequences are homologous with sequences that flank the gene; that is, one sequence is homologous with a region at or near the 5′ portion of the coding sequence of the nucleic acid and the other sequence is further downstream from the first. One skilled in the art would appreciate, based upon the disclosure provided herein, that the present invention is not limited to any specific flanking nucleic acid sequences. Instead, the targeting vector may comprise two sequences which remove some or all of, for example, one protein (i.e., a “knock-out” vector) or which insert (i.e., a “knock-in” vector) a nucleic acid encoding a second protein, or a fragment thereof, from or into a mammalian genome, respectively. The crucial feature of the targeting vector is that it comprise sufficient portions of two sequences located towards opposite, i.e., 5′ and 3′, ends of the desired open reading frame (ORF) in the case of a “knock-out” vector, to allow deletion/insertion by homologous recombination to occur such that all or a portion of the nucleic acid encoding the protein is deleted from a location on a mammalian chromosome.

The design of transgenes and knock-in and knock-out targeting vectors is well-known in the art and is described in standard treatises such as Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York), and the like. The upstream and downstream portions flanking or within the protein coding region to be used in the targeting vector may be easily selected based upon known methods and following the teachings disclosed herein.

Methods and compositions useful for maintaining mammalian cells in culture are well known in the art and detailed extensively elsewhere herein, wherein the mammalian cells are obtained from a mammal including, but not limited to, cells obtained from a mouse, a rat, a human, and the like.

Recombinant ES-like cells expressing a protein can be administered in ex vivo and in vivo therapies where administering the recombinant cells thereby administers the protein to a cell, a tissue, and/or a mammal. Additionally, the recombinant cells are useful for the discovery of ES-like cell differentiation and cellular processes. Ex vivo therapies are well known in the art and are described elsewhere herein.

As an example, a recombinant ES-like cell can be transplanted, infused or otherwise introduced into a developing organ of an animal in order to provide a detailed picture of the integration, migration and development of an ES-like cell in a developing organ. For example, recombinant ES-like cells expressing GFP can be injected into antenatal and/or neonatal mice or rats using techniques described above. The migration, development, and differentiation of the ES-like cells into such cells as neurons, oligodendrocytes, astrocytes, glial cells, and the like can be monitored by the expression of GFP in the ES-like cells as they mature into adult cells of the rodent brain. Such techniques will provide invaluable information for elucidating the development of the mammalian brain, as well as identifying the factors necessary for ES-like cell development.

In addition, the ES-like cells, identified by their expression of GFP or another tag molecule, can be analyzed as to the differentiated state they have obtained. That is, after a period of development in vivo, the transplanted ES-like cells can then be analyzed, either in vivo or ex vivo, to determine the differentiated phenotype. Such methods will provide the skilled artisan with information as to the regions of the brain and other organs and the extracellular cues that guide stem cell development in the developing organ. Such information will greatly increase the body of knowledge relating to the developmental and differentiation pathways taken by a stem cell in the body. Such techniques are useful not only for the brain, but for the developing spinal cord, eye, liver, kidney, heart, lungs, pancreas, and other cells and organs where understanding significant development can provide important potential therapeutics.

The skilled artisan would also appreciate, based upon the disclosure provided herein, that the present invention encompasses production of a skin stem cell that can be used in a wide variety of applications, including, but not limited to, cosmetics. This is because the common regenerative capacity of both Schwann cells and keratinocytes amply supports, in light of the data disclosed herein, that keratinocytes can be reprogrammed according to the methods disclosed elsewhere herein for producing ES-like stem cells using Schwann cells. Further, it has been demonstrated previously that dystroglycan is present in epithelial cells (see, e.g., Hemler, 1999, Cell 97:543-546; Spear, 1998, Science 282:1999-2000), and thus keratinocytes can be infected with M. leprae and used to produce ES-like cells.

Once skin stem-like cells are produced from an animal, the cells can be manipulated using methods well-known in the art (e.g., Okumura et al., 2003, Oncogene 22:4017-4026) to secrete important collagens, which, upon transplantation, can, among other things, regenerate aging skin, thus providing important novel therapeutics to treat or alleviate effects of aging on the skin of the animal. These methods further provide an improved therapy compared with prior art methods that simply apply collagens directly to the face. The present methods whereby skin stem-like cells producing collagens of interest are administered directly to the skin of an animal are a vast improvement to such prior art methods based on topical application of collagens to the face and other skin surfaces of an animal in need therefor. This is because one skilled in the art would readily appreciate, once armed with the teachings of the present invention, that skin is the most readily available tissue in the body. Therefore, transplantation of the skin stem cells to the skin of a recipient would be, in essence, a topical-based procedure (i.e., the outer layer of the skin would be affected and not much systemic involvement would be mediated by such topical-like transplantation procedure), which would make direct application of the skin stem-like cells possible.

One of skill in the art, when equipped with the present disclosure, will readily appreciate that the present invention comprises a method for providing a patient with either an autologous or allogeneic graft of ES-like cells. According to the present invention, differentiated human cells from any tissue type contacted with viable or irradiated M. leprae, or a component thereof, cultured for a period of time and with growth factors set forth herein, differentiated into another type of cell using techniques and reagents well-known in the art and those detailed elsewhere herein. Differentiation can take place either in vivo or in vitro, resulting in a ready supply of cells and tissues that can be used to treat a number of diseases, including, but not limited to spinal cord injury, Parkinson's disease, Alzheimer's disease, diabetes, multiple sclerosis, and the like.

The methods described above can include viable or non-viable M. leprae, as well as components of M. leprae. Irradiated non-viable bacteria and bacterial components are not infectious, and will not result in pathogenesis in patients receiving reprogrammed cells. M. leprae, as is well known in the art, is a pathogenic organism, and thus, the present invention further comprises methods for the dilution and eventual nullification of any viable M. leprae in reprogrammed cells. As demonstrated by the data disclosed, differentiated cells invaded by M. leprae exhibit the hallmark stem cell trait of continual proliferation without transformation. Thus, the present invention comprises contacting, and subsequent invasion of cells with viable M. leprae and expanding the ES-like cell population extensively, thus diluting the bacterial load to zero. Therefore, the final ES-like cells are bacteria-free and safe for transplantation and the presence of any viable M. leprae in a sample can be assessed to ascertain that no pathogenic bacteria are present in the sample.

In order to verify the safety of ES-like cells for transplantation, a variety of methods are known in the art for detecting M. leprae. PCR amplification of conserved sequences of M. leprae, such as the 16S ribosomal DNA sequences, can be used to demonstrate that the ES-like cells of the present invention are free from M. leprae. Commercial tests are available for the molecular detection of M. leprae (GENOTYPE™ Series, Hain Lifescience, Nehren, Germany), as well as conventional tests, such as the acid-fast smear (Difco, Detroit, Mich.) or immunofluorescence detection of PGL-1, as described below.

The present invention further comprises therapeutic methods comprising delivering viable or irradiated M. leprae, or components thereof to cells, either ex vivo or in vivo, using, among other things, liposomes. The present invention is useful in that liposomes can deliver M. leprae to any cell or tissue type by fusing with the cell membrane, thus delivering viable or irradiated M. leprae, or components thereof, to any cell type. While the present invention comprises infecting any cell type with M. leprae using dystroglycan/laminin interaction, liposomes offer the additional benefit of a avoiding any immune response to M. leprae in vivo and ex vivo. That is, M. leprae, when delivered in vivo or ex vivo, can result in an immune response in the individual or cell population. For example, without wishing to be bound by any particular theory, when the methods disclosed herein are used to reprogram ex vivo T-cells from an M. leprae-immune donor, contacting the cells with M. leprae may result in activation of the T-cells, possibly rendering them unsuitable for transplantation after reprogramming, or alternatively, activating gene transcription that will interfere with the reprogramming process. However, if viable or irradiated M. leprae, or components thereof, are administered via liposome, an immune response to the bacteria or bacterial components can be highly attenuated or ameliorated, and the T-cells can be reprogrammed. Thus, the present invention comprises reprogramming cells using liposome encapsulated M. leprae.

Properties of lipid formulations can vary depending on the composition of the starting material (cationic, anionic, neutral lipid species), however, the same preparation method can be used for all lipid vesicles useful in the present invention. Cationic, anionic and neutral lipid species are well known in the art and are available from a wide variety of sources (Avanti Polar Lipids, Inc., Alabaster, Ala.). The general elements of the procedure involve preparation of the lipid for hydration, hydration with agitation, and sizing to a homogeneous distribution of vesicles.

The lipids comprising the liposome are first dissolved and mixed in an organic solvent to assure a homogeneous mixture of lipids, usually with chloroform, chloroform:methanol mixtures, tertiary butanol, or cyclohexane until a clear lipid solution is obtained. Typically lipid solutions are prepared at about 10-20 mg lipid/ml organic solvent, although higher concentrations may be used if the lipid solubility and mixing are acceptable. Once the lipids are thoroughly mixed in the organic solvent, the solvent is removed to yield a lipid film. For small volumes of organic solvent (<1 ml), the solvent may be evaporated using a dry nitrogen or argon stream in a fume hood. For larger volumes, the organic solvent can be removed by rotary evaporation. The lipid film is thoroughly dried to remove residual organic solvent by placing the vial or flask on a vacuum pump for a period of time, usually overnight. The lipid solution is transferred to containers and frozen by placing the containers on a block of dry ice or swirling the container in a dry ice-acetone or alcohol (ethanol or methanol) bath. The container should be of the sort that can tolerate sudden and extreme temperature changes without cracking (e.g. PYREX). After complete freezing, the lipid cake is placed on a vacuum pump and lyophilized until dry (about 1-3 days depending on volume). The thickness of the lipid cake should be no more than the diameter of the container being used for lyophilization. Dry lipid films or cakes can be removed from the vacuum pump, the container close tightly and taped, and stored frozen until hydration.

Hydration of the dry lipid film/cake is accomplished by adding an aqueous medium to the container of dry lipid and agitating. The temperature of the hydrating medium should be above the gel-liquid crystal transition temperature (Tc or Tm) of the lipid with the highest Tc before adding to the dry lipid. After addition of the hydrating medium, the lipid suspension should be maintained above the Tc during the hydration period. For high transition lipids, this is accomplished by transferring the lipid suspension to a round bottom flask and placing the flask on a rotary evaporation system without a vacuum. Spinning the round bottom flask in the warm water bath maintained at a temperature above the Tc of the lipid suspension allows the lipid to hydrate in its fluid phase with adequate agitation. Hydration time may differ slightly among lipid species and structure, however, a hydration time of about 1 hour with vigorous shaking, mixing, or stirring is highly recommended. Allowing the vesicle suspension to stand overnight (aging) prior to downsizing may facilitate the sizing process and improve the homogeneity of the size distribution. Aging is not recommended for high transition lipids as lipid hydrolysis increases with elevated temperatures. The hydration medium is generally determined by the application of the lipid vesicles. Suitable hydration media include distilled water, buffer solutions, saline, and nonelectrolytes such as sugar solutions. Physiological osmolality (290 mOsm/kg) is recommended for in vivo applications. Generally accepted solutions with meet these conditions are 0.9% saline, 5% dextrose, and 10% sucrose.

The product of hydration is a large, multilamellar vesicle (LMV). Once a stable, hydrated LMV suspension has been produced, the particles can be downsized by a variety of techniques, including sonication or extrusion.

Disruption of LMV suspensions using sonic energy (sonication) typically produces small, unilamellar vesicles (SUV) with diameters in the range of 15-50 nm. The most common instrumentation for preparation of sonicated particles are bath and probe tip sonicators. Sonication of an LMV dispersion is accomplished by placing a test tube containing the suspension in a bath sonicator (or placing the tip of the sonicator in the test tube) and sonicating for about 5 to about 10 minutes above the Tc of the lipid. The lipid suspension should begin to clarify to yield a slightly hazy transparent solution. The haze is due to light scattering induced by residual large particles remaining in the suspension. These particles can be removed by centrifugation to yield a clear suspension of SUV. Mean size and distribution is influenced by composition and concentration, temperature, sonication time and power, volume, and sonicator tuning.

Lipid extrusion is a technique in which a lipid suspension is forced through a polycarbonate filter with a defined pore size to yield particles having a diameter near the pore size of the filter used. Prior to extrusion through the final pore size, LMV suspensions are disrupted either by several freeze-thaw cycles or by prefiltering the suspension through a larger pore size. This method helps prevent the membranes from fouling and improves the homogeneity of the size distribution of the final suspension. As with all procedures for downsizing LMV dispersions, the extrusion should be done at a temperature above the Tc of the lipid. Extrusion through filters with 100 nm pores typically yields large, unilamellar vesicles (LUV) with a mean diameter of 120-140 nm. Mean particle size also depends on lipid composition and is quite reproducible from batch to batch. Thus, choosing a pore size similar to that of M. leprae bacterium, or components thereof, will yield liposomes capable of encapsulating M. leprae for the methods of the present invention. Alternatively, large liposomes capable of encapsulating a bacteria, or components thereof, are available commercially (Lipoxen, London, UK).

Thus, the skilled artisan, based upon the disclosure provided herein, would appreciate that because the M. leprae cell wall comprises high amounts of lipids, and glycolipids, and they are known to play biological roles. These cell wall components can also play a significant role in the reprogramming process. Accordingly, the skilled artisan would understand that purified cell wall lipids, such as PGL-1 and LAM, can be delivered to cells using liposomes.

V. Methods for Affecting/Modulating Cell Proliferation

The skilled artisan, when armed with the present invention and the disclosures herein, will readily appreciate that the present invention encompasses novel methods and compositions for modulating/affecting the mammalian cell cycle, specifically, for increasing cellular proliferation without transformation of a cell. That is, the data disclosed herein demonstrate, for the first time, that intracellular M. leprae, or a component thereof, including the cell wall fraction or components thereof, is capable of initiating and regulating the mammalian cell cycle, i.e., likely via phosphorylation of an extracellular signal regulated kinase (ERK1/2) and protein kinase glycogen synthase kinase-3-beta (GSK-3-beta). Further, unlike conventional cell-signaling pathways, the present invention is based in part on the discovery that phosphorylation of ERK1/2 and GSK-3-beta is not through the phosphatidylinositol-3′-kinase pathway (PI3K) or the Ras>Raf>MEK1/2 pathway, but rather through a novel signaling pathway (NSP) previously unknown. This signaling was identified as p56LCK-ser 158-mediated phosphorylation of Erk1/2 via PKC-dependent manner that bypasses the canonical growth factor-induced receptor-mediated Ras, Raf, Mek1/2 and Erk1/2 pathway. Sustained activation of the latter usually induces cell transformation whereas the former, novel pathway does not cause transformation despite continuous proliferation.

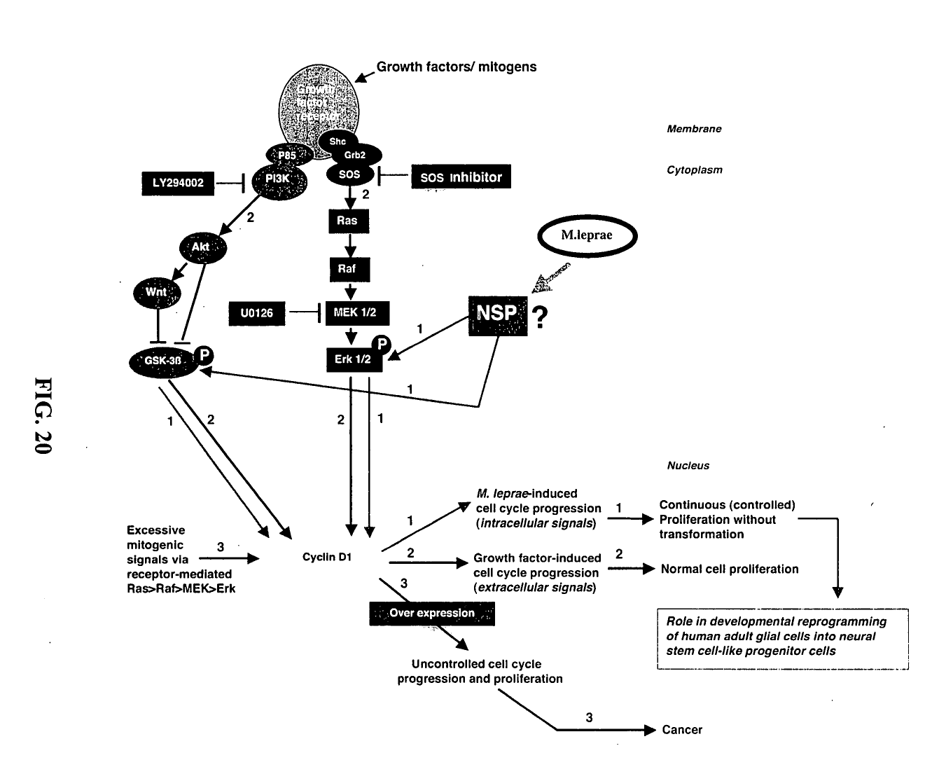

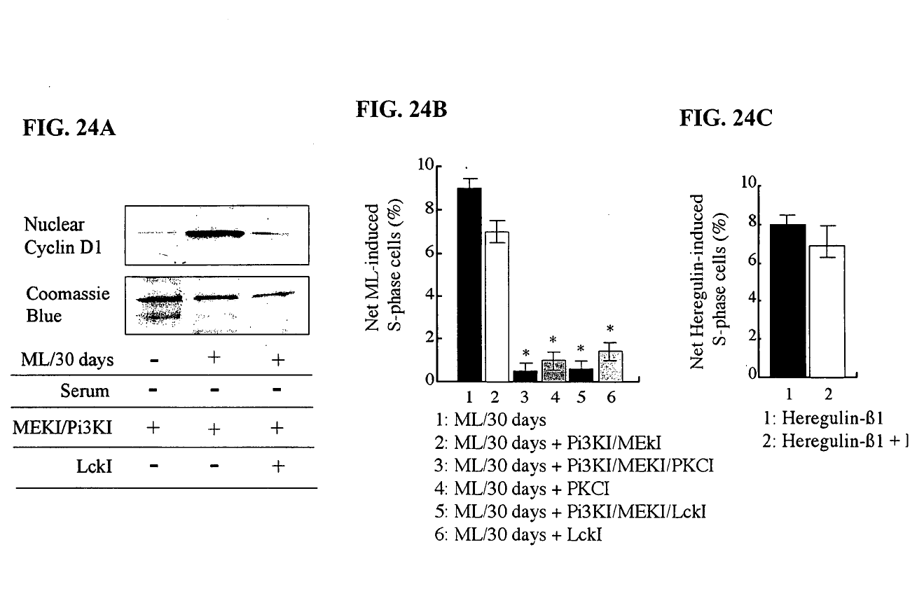

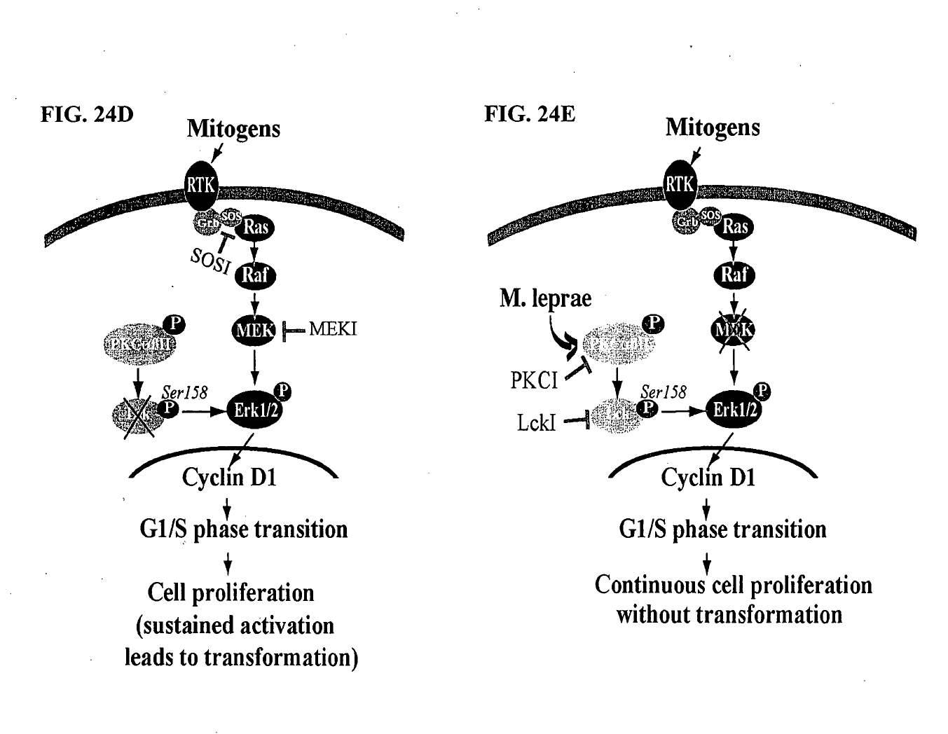

Without wishing to be bound by any particular theory, as illustrated in FIG. 20, and FIG. 24D, cellular proliferation is controlled through a growth factor or mitogen binding to an extracellular receptor and initiating a cascade of phosphorylation and activation of signaling proteins that eventually results in the phosphorylation and activation of cyclin D1, which when bound to the appropriate cyclin dependent kinase (CDK), results in progression through the G1 phase of the cell cycle and cellular proliferation. When dysregulated at any one of multiple points along this pathway, the cell cycle progresses uncontrollably and cancer results. However, the data disclosed herein disclose a novel pathway relating to intracellular signaling which is independent of the MEK1/2 pathway (see FIG. 24E). Indeed, the novel pathway comprises phosphorylation by serine 158-Lck, which had not been detected in differentiated cells prior to the present invention. Thus, the data disclosed demonstrate, surprisingly, that phospho-p56Lck/Ser-158 plays a role in MEK/Pi3k-independent NSP and is a major component of the pathway.

One skilled in the art would appreciate, based upon the disclosure provided herein, that because phospho-Lck/Ser-158 is a major component of the NSP pathway, the pathway can be regulated using known inhibitors and inducers of Lck phosphorylation.

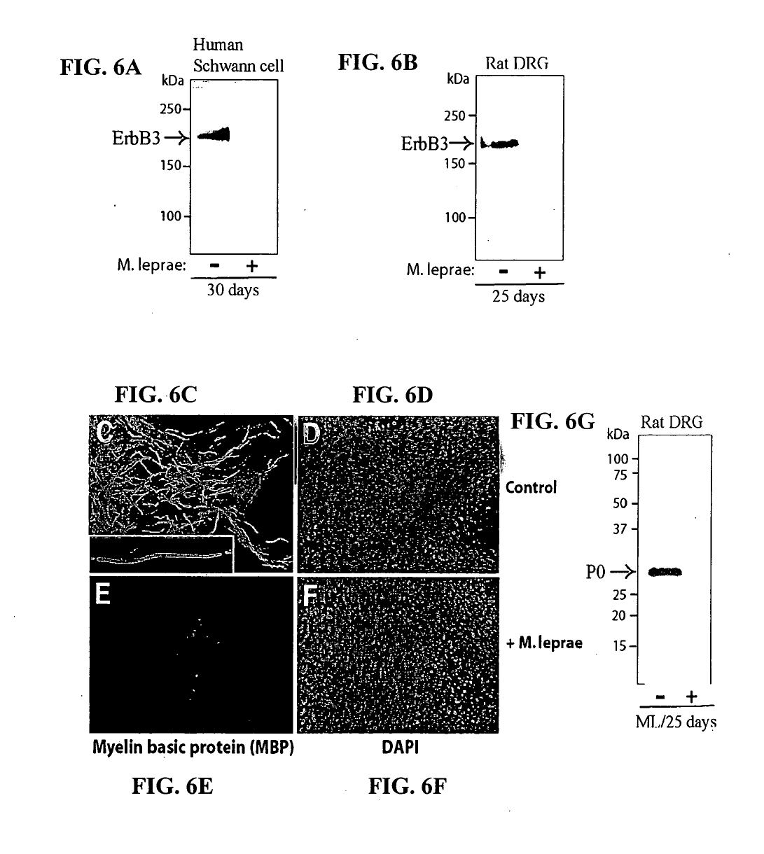

During development many signaling pathways are activated and some are down-regulated and others are re-activated, while yet others, that appear in the embryonic stage, do not activate or express in adult differentiated cells. Lck/Ser-158-mediated Erk1/2 activation is not detected in adult differentiated cells, because it was not detectable when activated by Schwann cell mitogen heregulin-beta-1. The latter, upon binding to heregulin receptor ErbB3, induces canonical MEK-dependent Erk1/2 activation. It should be noted that 30 day-infected human Schwann cells do not express ErbB3 protein (see FIG. 6), and therefore, heregulin action cannot be accomplished in infected cells). On the other hand, Lck/Ser-158-mediated Erk1/2 activation is likely to occur in immature or progenitor cells. It is possible that by activation of Lck/Ser-158-mediated Erk1/2 phosphorylation, intracellular M. leprae activate a signaling pathway that normally operates in stem cells, and induction of such signaling in adult cells can initiate reprogramming. Activation of such signaling can be a mechanism by which M. leprae reprogram adult cells to stem cell-like cells.

Another implication of the data disclosed herein demonstrating a novel Lck?ser-158-mediated Erk1/2 pathway in adult cells is in cancer. This is because activation of this pathway induced continuous cell proliferation without transformation. Without wishing to be bound by any particular theory, it may be that therapeutic up-regulation of this pathway can prevent development of cell transformation and/or cancer formation.

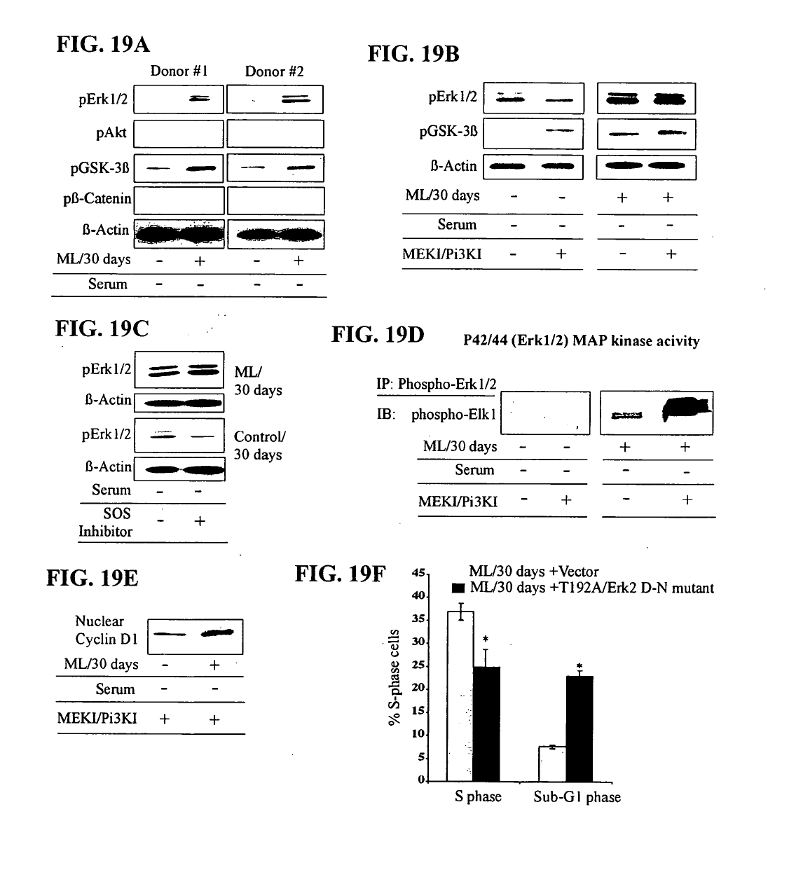

The present invention comprises a method of regulating the mammalian cell cycle and inducing cellular proliferation without transformation. While not wishing to be bound by any particular theory, this is because intracellular M. leprae, or components thereof, initiate a novel pathway resulting in the phosphorylation of GSK-3-beta (GSK3β) and ERK1/2, yet this phosphorylation proceeds despite inhibition of the only known upstream kinases, MEK and Pi3-kinase, for GSK3β and Erk1/2 phosphorylation. That is, intracellular M. leprae can activate previously unidentified MEK- and Pi3Kinase-independent signaling pathways to phosphorylate GSK3β and Erk1/2 (see FIG. 19F and FIG. 20). Thus, introduction of M. leprae that is viable, non-viable, or a component thereof, into a cell mediates/activates a novel signaling pathway disclosed herein, mediating, in turn, transcription of cyclin D1 and assembly of a cyclin D1/CDK4 complex that subsequently drives cellular proliferation without causing the cell to develop a transformed phenotype.

The method for regulating the mammalian cell cycle and inducing cellular proliferation without transformation comprises contacting a cell with viable or irradiated M. leprae, or a component thereof, and incubating the cell for a period of time as set forth elsewhere herein. As described elsewhere herein, methods for contacting or infecting a cell with M. leprae, or a component thereof, are well known in the art and some are disclosed and exemplified herein.

Methods for measuring progression through the cell cycle and for assessing the level of cellular proliferation are well known in the art and are also described elsewhere herein. These methods include, but are not limited to, measuring the phosphorylation state of cyclins and CDKs by Western blotting, FACS analysis, genearrays, size differentiation on an SDS-PAGE gel, mass spectrometry, assessing the stage of the cell cycle that the cell is in and determining the percentage of cells that are in each phase of the cell cycle, and the like.

Further, cellular proliferation can be measured by simply observing cells treated in the manner of the present invention compared to cells not contacted with M. leprae, or a component thereof. Cells that are induced to proliferate will increase in number over a given period of time when compared to cells that are not proliferating. Methods for measuring cell proliferation are described elsewhere herein and are known in the art. In addition, proliferating cells of the present invention will not demonstrate transformed phenotypes, such as, but not limited to, lack of contact inhibition and plaque formation, and therefore, a control comprising a transformed cell type, such as HeLa cells, MCF7 breast cancer cells, SKBR-3, or any other transformed cell type known in the art can be used to compare the proliferation of the cells of the present invention with both non-proliferating cells and “transformed” cells.

Unlike prior art methods available for accelerating cell cycle progression, such as overexpression of G1 phase regulators or signaling molecules, such as, ras and ERK and Pi3K/Akt, which usually cause cell transformation, the novel methods set forth herein do not cause transformation.

The present invention also encompasses novel methods for elucidating the common characteristics of cancer cells and stem cells, and thus elucidating new treatments for cancer. Cancerous cells and stem cells share the common feature that both cell types proliferate and both types of cells demonstrate the upregulation of cyclin D1 and other cell cycle regulators, including GSK3β and Erk1/2. Therefore, the novel pathway by which M. leprae phosphorylate GSK3β and Erk1/2 in order to induce nuclear accumulation of cyclin D1 (requirement for S-phase progression) (FIGS. 19F and 20) described herein offers a new and valuable tool in understanding where the beneficial aspects of stem cell proliferation end, and where the malignant transformation of cancer begins.

While not wishing to be bound by any particular theory, the data demonstrate that the cellular proliferation induced by M. leprae is mediated through a novel signaling pathway, and the lack of transformation is most likely due to MEK/Pi3K-independent phosphorylation of Erk1/2 and GSK3β and subsequent transcriptional control of gene expression. Thus, cellular components transcribed, translated, phosphorylated or otherwise activated directly or indirectly by infection with M. leprae are valuable for defining the processes that lead to cellular proliferation without transformation and represent important potential targets for development of therapeutics such as, but not limited to therapeutics for inhibiting proliferation of cancer cells, and the like.

The methods of the present invention can be used to elucidate the differences between normal, proliferating cells and cancer cells, and from this information, the mechanisms of abnormal cancerous cell proliferation can be identified and therapies can be designed that specifically target transformed cells while not having normal proliferating cells. That is, therapeutic up-regulation of novel MEK/Pi3-kinase-independent signaling pathway can be used for prevention of cancer or progression of cancer.

In order to identify genes modulated by intracellular M. leprae, or components thereof, the skilled artisan can use methods described elsewhere herein, such as Western blotting, analysis by genearrays, inhibiting specific members of the phosphorylation cascade, and the like. As a non-limiting example, a cell infected with M. leprae is lysed in an detergent containing buffer to obtain nuclear extracts. Control cells of the same type are lysed in an identical fashion. If available, a transformed cell (for example a cell transfected with the SV40 T-antigen or a known oncogene) of the same type is also lysed under identical conditions. The lysed cell supernatant is further sheared through sonication or by passing the lysed cell supernatant through a small bore needle, such as a 25 gauge needle. The sheared sample is run on an SDS-PAGE gel under conditions well known in the art. The gel is transferred to a nitrocellulose or PVDF membrane, blocked and probed with a primary antibody to specific members of the tyrosine kinase cascade, or other prominent molecules related to cell cycle progression and cellular proliferation, such as phosphorylation of Erk1/2. After detection of antibody binding, the differences among control, transformed, and M. leprae-infected cells will be apparent. For example, if Erk1/2 is phosphorylated in the presence of MEK and Pi3Kinase inhibitors this can indicate the difference between the healthy, proliferating cells of the present invention and malignant cancerous cells, since cancer cells inhibit Erk1/2 phosphorylation in the presence of MEK and Pi3Kinase inhibitors (both MEK and Pi3Kinase inhibitors are currently in clinical trails in cancer patients).

The skilled artisan will recognize that the method described above is not solely limited to Erk1/2 phosphorylation, but can be altered with little or no experimentation to embrace many cell cycle proteins well known in the art and described elsewhere herein. Primary antibodies to a great number of both phosphorylated and unphosphorylated cell cycle-related proteins (e.g., GSK3β, cyclin D1, Rb, and E2F) are available commercially and can be used in the afore-mentioned assays.

Further, as described elsewhere herein, gene arrays can be used to detect differences in gene transcription between control, transformed, and proliferating cells. Gene arrays are well known in the art, and materials and methods for their use are described in detail below. As a non-limiting example, the mRNA of a control, transformed, and M. leprae-induced proliferating cell can isolated using any number of commercial kits or reagents. The mRNA, after reverse transcription, can be hybridized to a gene array. Using software provided by the manufacturer, the expression of genes in control, transformed, and proliferating cells can be analyzed and provide the skilled artisan with the differences in transcriptional control between healthy and cancerous cells.

Thus, when armed with the present disclosure and the methods disclosed above and elsewhere herein, the skilled artisan will readily appreciate that the present invention comprises a method for elucidating the differences between proliferating cells and cancerous cells. Induction of Erk1/2 and GSK3β phosphorylation are common to both Schwann cells infected with M. leprae and cancer cells, and both M. leprae and cancer induce cyclin D1 nuclear accumulation and subsequent cell proliferation. In cancer cells such events lead to uncontrolled proliferation, whereas in M. leprae infected Schwann cells such signaling produced highly controlled cell proliferation. The latter resulted via a MEK/PI3Kinase independent pathway whereas the former is via a MEK/PI3Kinase dependent pathway, which leads to uncontrolled proliferation. Therefore, a M. leprae-induced novel signaling pathway can contribute to continuous cell proliferation without transformation, a feature that is characteristic of stem cells.

The present invention also encompasses a method of inducing cyclin D1 in a cell by MEK-Pi3K-independent phosphorylation of Erk1/2 and/or GSK3-beta. This is because it is amply demonstrated by the data disclosed herein that contacting an adult cell with an M. leprae bacterium, or a component thereof, induced cyclin D1 expression and accumulation of the protein in a cell via a novel MEK-Pi3K-independent phosphorylation signaling pathway. This method is useful in that the data disclosed herein further indicate that such induction of cyclin D1, independent of MEK-Pi3K phosphorylation, mediates, among other things, cell proliferation without unwanted cellular transformation.

Similarly, the invention includes a method of inducing Erk-1 phosphorylation, GSK3beta phosphorylation, or both, via MEK-Pi3K-independent phosphorylation. This is because the data disclosed herein demonstrate that phosphorylation of these proteins occurs via a novel signaling pathway that is MEK-Pi3K-independent when a cell is contacted with an M. leprae bacterium, or a component thereof. This novel signaling pathway is useful in that the cells undergo reprogramming and proliferation without detectable transformation, thereby providing an important source of cells for use in cell-based therapies and overcoming previous obstacles which hampered such therapies until being overcome by the novel methods disclosed herein.

The novel signaling pathway disclosed herein normally does not appear to function in adult Schwann cells or in other differentiated adult cells but can be induced in response to certain stimuli. For instance, the NSP can be induced by intracellular M. leprae and MEK and Pi3K inhibitors that shut down the critical signaling pathways involved in normal cellular processing. The novel pathway appears to play a standby role for cell survival and is likely to be induced during development, but turns off as cells acquire differentiated stage. Therefore, without wishing to be bound by any particular theory, induction of this pathway, by e.g., intracellular M. leprae, can change the downstream transcriptional regulation and early gene expression and thereby change the fate of adult cells into an immature stage.

VI. Methods for Identifying an M. leprae Component Relating to Cell Reprogramming and the Like

The present invention includes methods for identifying a M. leprae component involved in reprogramming of a cell into an ES-like cell. The sequencing of the human genome (Venter et al., 2001, Science 291: 1304-1351), and the M. leprae. genome (Cole et al., 2001, Nature 409:1007, GenBank Acc. No. AL450380) demonstrated the high degree of skill in the art. Knowledge of these genomes can facilitate the identification of an isolated M. leprae component that mediates the novel cellular reprogramming disclosed herein.

Thus, the present invention comprises a method for assessing the effects of M. leprae proteins, specifically cell wall proteins, secreted proteins and related components in a human cell or in an in vitro protein expression system to identify interactions and functions. This is because, as demonstrated by the data disclosed herein, components of the cell wall and/or a secreted protein of M. leprae, when contacted with a human differentiated cell, results in the reprogramming of a differentiated cell into an ES-like cells. The present data demonstrate that not only viable M. leprae, but irradiated M. leprae is capable of reprogramming cells. Since irradiated M. leprae do not replicate, the data disclosed herein demonstrate that a component of the M. leprae cell wall is responsible, at least in part, for the novel effects disclosed herein. Many genes encoding cell wall components and secreted proteins have been identified, and have been described in, e.g., Cole et al., 2001, Nature 409:1007).

One approach is to use phospho-antibody assays known in the art and currently available for detecting phosphorylated signaling proteins in infected compared with control cells and to analyze which signaling proteins are phosphorylated in reprogrammed cells (e.g., differential expression) and their connection to existing signaling pathways and gene expression.

Further, methods for analyzing the interaction of two proteins are well known in the art. The skilled artisan can express a protein from a cell, for example a human Schwann cell, a portion of a cell wall, and/or a secreted protein from M. leprae. Then, methods for the expression and analysis of the interaction between two proteins can be used, such as, but not limited to, the yeast two-hybrid method as described in U.S. Pat. Nos. 5,283,173 and 5,468,614, which method is available commercially in kit form. As a non-limiting example, in order to analyze the interaction of the M. leprae cell wall protein ML-LBP21 (Shimoji et al., 1999, Proc. Natl. Acad. Sci. USA 96: 9857-9862) with the human homeobox transcription factor HSIX1 (Ford et al., 1998, Proc. Natl. Acad. Sci. USA 95: 12608-12613), both sequences are expressed in a yeast two-hybrid system according to the manufacturer's instruction. A positive interaction between the M. leprae cell wall protein and a signaling molecule or transcription factor indicates that the M. leprae protein may play a role in activating signaling and transcriptional regulation and may play a role in the reprogramming process. The preceding method should not be construed as being limited to the proteins detailed above. The present method is useful for determining the reaction between any M. leprae protein derived from the cell wall, membrane, cytoplasm, or secreted by the bacterium.

A hierarchical list of M. leprae genes and their sequences is available publicly at the website of The Wellcome Trust Sanger Institute. Thus, the skilled artisan, when equipped with the present disclosure and the methods disclosed herein, can identify M. leprae reprogramming genes for use in creating ES-like stem cells. Further, the present invention can be applied to any protein differentially expressed in a human cell, preferably signaling molecules, cell cycle regulatory proteins, and transcription factors disclosed herein. The skilled artisan, when equipped with the present invention and the methods disclosed herein, will be able to readily identify M. leprae proteins that are capable of reprogramming cells.

As a non-limiting example, a protein that is associated with or mediates the reprogramming ability of M. leprae can be identified using the methods described elsewhere herein. The component can be either isolated in its native form using M. leprae fractionation techniques disclosed elsewhere herein, or cloned and expressed using traditional and well known molecular biology techniques described in, for example, Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York). Such a protein can comprise a cell wall protein, a membrane protein, a cytoplasmic protein, a nuclear protein, or another M. leprae protein identified by the methods disclosed herein.

Briefly, M. leprae bacteria can be disrupted using a variety of methods including sonication, phenol extraction, fractionation with a French Press, or other methods well known in the art. Additionally, the culture filtrate of M. leprae can be fractionated in order to identify and isolate secreted M. leprae proteins. The M. leprae protein can then be loaded and run on an SDS-PAGE gel to separate proteins, glycoproteins, and other components by size. The proteins are then size fractionated using a whole gel eluter, such as the BioRad whole gel eluter (BioRad, Hercules, Calif.). The whole gel elution process allows the preparation of size fractionated M. leprae components into aliquots comprising proteins, glycoproteins, and other M. leprae components confined to a small range within about 5 kDa of each other. As an example, after elution, a small group of proteins comprising a range of about 30 kDa to about 35 kDa, another small group of proteins comprising a range from about 25 kDa to about 20 kDa, and the like, can be eluted and used to reprogram cells. The proteins, after elution, are relatively free of SDS and other contaminants, and can be used directly in the methods disclosed herein. Further, because of the limited number of proteins existing within a small (5 kDa) size range, the protein mediating the desired reprogramming effect can then be isolated and characterized by methods well known in the art in order to further investigate its reprogramming abilities and properties.

For instance, the protein can be introduced to a differentiated cell to be reprogrammed using a cationic lipid-based carrier system. Briefly, cells are isolated from a patient or cell line as described above, and grown to approximately 50-60% confluency. The protein is diluted in a physiologically acceptable carrier, such as phosphate buffered saline (PBS), and the protein/PBS solution is used to hydrate the dried cationic lipid carrier. The carrier/protein solution is then mixed well and diluted further with serum-free medium. The carrier/protein solution is then applied to the cells to be reprogrammed and the cells are incubated in a medium comprising the solution.

In order to monitor the progress of the protein transfection protocol, a control protein that, such as a fluorescent protein, can be transfected along with the M. leprae protein and observed under ultraviolet light. In order to account for variations in protein hydrophobicity and charge dynamics, the pH of the buffer used may be altered to accommodate protein variations. Methods and compositions for protein transfection are well known in the art and are available commercially from a variety of sources (Pierce Biotechnology, Rockford, Ill.). It would be understood by the skilled artisan, armed with the teachings provided herein, that the component of interest can be administered to a cell by protein transfection using purified M. leprae cell walls or secreted proteins.

The present invention further comprises obtaining soluble and insoluble cell wall fractions and membrane proteins of M. leprae and transfecting them into differentiated cells in order reprogram a differentiated cell into an ES-like cell state. M. leprae cell wall fractions and membrane proteins with reprogramming capabilities can be identified and isolated using methods described herein. Briefly, bacterial cultures are harvested as described below and probe-sonicated in a buffer containing protease inhibitors (e.g., 50 mM Tris, 10 mM MgCl₂, 1 mM EGTA, 1 mM PMSF, 0.02% NaN₃, pH 7.4). The sonicates are centrifuged, initially at about 23,000×g to remove cell wall debris. The cell wall fractions can then be used for dedifferentiating a cell.

Methods to produce an M. leprae membrane fraction are well known in the art, and include sonication and use of detergents known in the art. A cell wall fraction, a membrane protein fraction, or other component of M. leprae can then be administered to a differentiated cell as described elsewhere herein, and incubating the cell with the M. leprae, or component thereof.

Additionally, the interaction of M. leprae, or component thereof, can be assessed for their ability to interact directly with differentiated cell DNA using a procedure similar to the yeast two-hybrid assay. Yeast one-hybrid techniques are well known in the art, and provide an indicator of a protein-DNA interaction (Clontech, Palo Alto, Calif.). Thus, the present invention comprises a method for identifying reprogramming factors in M. leprae that interact directly with differentiated cell DNA.

The present invention further includes a method of identifying a compound that affects cellular proliferation. The method comprises contacting a cell with an M. leprae component and comparing the level of proliferation in the cell so contacted with the proliferation in an otherwise identical cell not contacted with the component. If the level of proliferation is higher or lower in the cell contacted with the component compared to the level of proliferation in the otherwise identical cell not contacted with the component, this is an indication that the component affects cell proliferation. Thus, the routineer would appreciate that the invention encompasses methods for so affect cell proliferation. This is because, as demonstrated, the data disclosed herein, contacting a cell with M. leprae (viable or killed), or a component (i.e., a cell wall, a portion of a cell wall, and the like) thereof, mediates cell proliferation without causing transformation (i.e., loss of contact inhibition, among other things) and effect the progress of the cell through the cell cycle.

The invention encompasses methods to identify a compound that reprograms a cell. One skilled in the art would appreciate, based upon the disclosure provided herein, that assessing the reprogrammed status of a cell can be performed using gene arrays, morphologic alterations, proliferation without transformation, and the like, such that the method can identify a compound that selectively affects cellular reprogramming. Such compounds are useful for creating ES-like cells. One skilled in the art would understand that such compounds can be useful for treating a disease, such as diabetes, multiple sclerosis, Parkinson's disease, Alzheimer's disease, and the like. Thus, the skilled artisan would appreciate, based on the disclosure provided herein, that it may useful to identify compounds that reprogram cells.

Indeed, as exemplified elsewhere herein, following the methods of the invention, a surprising major component of a novel signaling pathway that mediates reprogramming of a differentiated cell has been identified, e.g., Lck/Ser-158. While the invention is not limited to this component, the data disclosed herein demonstrate that the methods of the invention can be used to identify useful components that can mediate the desired cell reprogramming of the invention.

The invention encompasses a method comprising identifying a nucleic acid encoding a M. leprae protein that when expressed or otherwise present in a differentiated cell, results in the reprogramming of the differentiated cell. The nucleic acid can be identified according to the methods of the present invention described elsewhere herein. The nucleic acid can be expressed in a plasmid, viral vector, or other type of vector. The vector comprising an M. leprae nucleic acid is transfected into a differentiated cell using techniques well known in the art and/or described elsewhere herein, and/or such methods as are developed in the future. As an example, the vector comprising an M. leprae nucleic acid is introduced into a cell through techniques such as electroporation, cationic lipid transfection, microparticle bombardment, and the like. Techniques for constructing and transfecting vectors, plasmids, and other nucleic acid delivery vehicles are well known in the art, are described elsewhere herein, and in standard treatises such as Sambrook et al. (1989, Molecular Cloning: A Laboratory Manual, Cold Spring Harbor Laboratory, New York), and Ausubel et al. (1997, Current Protocols in Molecular Biology, John Wiley & Sons, New York).

The differentiated cell can be transiently transfected with a plasmid encoding an M. leprae protein. The nucleic acid need not be integrated into the cell genome, nor does it need to be expressed in the cell. Moreover, the cell may be a eukaryotic cell and the invention should not be construed to be limited to any particular cell line or cell type. Such cells include, but are not limited to, Schwann cells, keratinocytes, and other cell types detailed above.

Further, it is important to note that the purpose of transgene-comprising, i.e., recombinant, cells should not be construed to be limited to the generation of reprogrammed cells. Rather, the invention should be construed to include any cell into which a nucleic acid encoding a M. leprae reprogramming gene(s) is introduced, including, without limitation, a eukaryotic cell comprising an isolated nucleic acid encoding a M. leprae reprogramming component.

The invention includes a eukaryotic cell which, when the exogenous nucleic acid of the invention is introduced therein, and the protein encoded by the desired nucleic acid is expressed therefrom, where it was not previously present or expressed in the cell or where it is now expressed at a level or under circumstances different than that before the transgene was introduced, a benefit is obtained. Such a benefit may include the fact that there has been provided a system wherein the expression of the nucleic acid of interest can be studied in vitro in the laboratory or in a mammal in which the cell resides, a system wherein a cell comprising the introduced gene can be used as research, diagnostic and therapeutic tools, and a system wherein non-human mammalian transgenic models are generated which are useful for the development of new diagnostic and therapeutic tools for selected disease states in a mammal. Further, the recombinant cell comprising the exogenous nucleic acid encoding an M. leprae component that mediates de-differentiation is useful in that the cell can be de-differentiated into an ES-like cell and used for, inter alia, regenerative medicine therapies.

A cell expressing an isolated nucleic acid encoding an M. leprae de-differentiation and/or reprogramming component can be used to provide a reprogrammed, re-differentiated, and/or trans-differentiated cell to a tissue, or whole mammal, where such a cell can be useful to treat or alleviate a disease, disorder or condition associated with a dysfunctional cell. Such diseases, disorders or conditions can include, but are not limited to, Alzheimer's disease, Parkinson's disease, multiple sclerosis, spinal cord or other CNS injury, diabetes, heart disease, and the like. Therefore, the invention includes a cell expressing a M. leprae reprogramming gene(s) to provide a reprogrammed, re- and/or trans-differentiated cell useful to treat or alleviate a disease, disorder or condition.

Recombinant cells expressing a M. leprae reprogramming component can be administered in ex vivo and in vivo therapies where administering the recombinant cells thereby administers the reprogrammed cell to a tissue, and/or a mammal. Additionally, the recombinant cells are useful for the discovery of a reprogramming component, signaling pathway, and the like.

The present invention further encompasses a method for culturing and propagating an ES-like cell generated according to the methods of the present invention. That is, as described below, the present invention comprises a method for expanding a population of ES-like cells in order to form a library of such cells, and/or for use in transplantation, infusion, or ex vivo therapies to treat diseases including, but not limited to diabetes, Alzheimer's disease, multiple sclerosis, Parkinson's syndrome, spinal cord injuries, as well as to replace skin lost in burns or due to melanoma, or as a method to combat the appearance of aging. Further, an ES-like cell can be expanded to produce large numbers of cells that can be re-differentiated to produce differentiated cells of the same, or a trans-differentiated to produce differentiated cells of a different, type than the original cell contacted with M. leprae. In this way, large numbers of differentiated cells can be produced of a cell type that cannot readily be expanded to produced large numbers of cells, thereby providing a crucial improvement that allows production of large numbers of a cell type of interest even where the cell does not typically proliferate when it is in the differentiated state.

The present invention further encompasses another important application of ES-like cells and that is the uncovering the mechanisms of genetic diseases and in generating sources of normal and impaired tissues for use in drug discovery. ES-like cell lines can be generated by reprogramming adult cells from a wide range of patients afflicted with, among other things, cancer, heart diseases and neurodegenerative diseases, using the methods disclosed herein. An immediate application of this invention is the ability of generating stem cell-like cell lines derived from diseased tissues where the disease has a genetic basis, such as motor neurons from amyotrophic lateral sclerosis (ALS), or cardiac muscles from heart disease patients. ES-like cells generated from these patients can be used to identify cellular processes/pathways that go awry in the diseased cells, and to design high-throughput screens to identify molecules that contribute to the disease or halt disease progression. Because existing human embryonic stem cell lines derived from human embryos do not harbor the mutation that causes these diseases, these embryonic ES cells cannot be used for studying such human diseases. Moreover, existing human ES lines do not reflect the genetic diversity of the overall population. Therefore, reprogramming adult cells from patients known to be afflicted with a disease, disorder or condition of interest, and reprogramming them to ES-like cells provides an enormous advantage over existing ES-cell technology for curing human diseases.

As demonstrated by the data disclosed herein, a differentiated cell of the present invention, when treated according the methods of the invention, is reprogrammed into a precursor de-differentiated state. That is, a terminally differentiated cell can be reprogrammed to an ES-like state in which the cell has pluripotent capability. The cell can then be reprogrammed into various cell types dependent on the signals provided to the ES-like cell. The methods of the present invention are particularly useful to the generation of neurons and oligodendrocytes. This is because the present disclosure demonstrates a method for, among other things, reprogramming a Schwann cell into a neural ES-like cell. The data disclosed herein show that the neural stem cells developed according to the methods of the present invention exhibit most of the characteristics (genetic, immunological and phenotypical) of neural stem cells isolated from fetal tissue and other sources. Surprisingly, the data disclosed herein demonstrate that neurospheres derived from Schwann cells reprogrammed by contact with viable or irradiated M. leprae, or a component thereof, are capable of self-renewal without transformation, are capable of re-differentiation into neuron-like cells and oligodendrocytes, and express a significant portion of the recognized markers of both neural stem cells and ES cells in general, all of which are characteristics of neural stem cells. In fact, reprogrammed human Schwann cells of the present invention upregulate about 47% of the mouse neural stem cell specific genes known, and about 31% of mouse ES cell specific genes, including cyclin D1, the characteristic stem cell signature gene.

The skilled artisan, when equipped with the teachings provided herein, will readily appreciate that in order to propagate and expand a population of ES-like cells, the cells can be grown in any suitable culture medium according to methods well-known in the art for culturing ES-line cells and/or ES cells. Such media suitable for the maintenance, propagation, and progenitor cell formation of ES-like cells and other stem cells is referred to herein as “progenitor media.” See, e.g., International Publication Nos. WO 93/01275, and WO 94/16718, both incorporated herein by reference.

The pH of the culture medium is typically between 6-8, preferably about 7, most preferably about 7.4. Cells are typically cultured between about 30-40° C., preferably between about 32-38° C., most preferably between about 33-37° C. Cells are preferably grown at about 5% to about 7% CO₂. Cells can be grown in suspension culture or as adherent cultures, depending on the propensity of the cells (see, e.g., U.S. Pat. Nos. 5,750,376 and 5,753,506).

Nutrient cell growth media are well known in the art and include the formulations presented below, and further includes modifications thereto that will readily become apparent to one of skill in the art, based upon the disclosure provided herein.

As an example, a progenitor media for the maintenance of ES-like cells, especially ES-like neural cells can comprise Dulbecco's Modified Eagles Medium (DMEM) plus F 12 medium (1:1) supplemented with N2 medium (Gibco, La Jolla, Calif.), bFGF (10-20 μg/ml) (Calbiochem, San Diego, Calif.), heparin (8 μg/ml) and/or EGF (10-20 μg/ml). The ES-like cells can be plated on uncoated tissue culture dishes, and the medium is changed about every five days.

As a further example, ES-like cells can be grown in the following progenitor media: 1×DMEM/F-12 0.5-2.0×, 0.6% w/v glucose, 2 mM glutamine, 3 mM NaHCO₃, 5 mM HEPES, 100 μg/ml apo-human transferrin, 25 μg human insulin, 60 μM putrescine, 30 nM, 20 nM progesterone, 20 ng/ml human EGF, 20 ng/ml human bFGF, 10 ng/ml human LIF, 2 μg heparin, and 5% CO₂. Additional progenitor media formulations and growth conditions for the maintenance/propagation of ES-like cells are well known in the art and are described in, for example, U.S. Pat. No. 6,528,306.

ES-like skin cells, such as keratinocytes, generated according to the methods of the present invention can be maintained/propagated in a suitable progenitor media as is well known in the art. Such progenitor media can be made according to the formulations of Rheinwald and Green, supra, on irradiated Swiss 3T3-J2 feeder layers in DMEM containing 10% FCS, 20 ng/ml epidermal growth factor, 0.4 μg/ml hydrocortisone, and 10 ng/ml cholera toxin. Keratinocytes can be passaged after removing the feeder cells with 0.02% EDTA. Alternatively, commercial formulations for the maintenance/propagation of ES-like keratinocytes or other skin cells are available (Keratinocyte Medium Kit, Sigma, St. Louis, Mo.).

In general, methods for the maintenance/propagation and/or expansion of ES or ES-like cells are well known in the art and are described in, for example, U.S. Pat. No. 6,200,806. Formulations for progenitor media for the maintenance and propagation of ES and ES-like cells can include 80% DMEM with no pyruvate and high glucose, 20% fetal bovine serum (FBS), 0.1 mM β-mercaptoethanol, 1% non-essential amino acid stock.

Preferably, fetal bovine serum batches are compared by testing clonal plating efficiency of low passage ES or ES-like cell lines. FBS batches should be compared because it has been found that batches vary dramatically in their ability to support ES and ES-like cell growth, but any other method of assaying the competence of FBS batches for support of embryonic cells can work as an alternative.

ES cells are often cultured on a confluent layer of murine embryonic fibroblasts in the presence of progenitor medium. Embryonic fibroblasts are preferably obtained from 12 day old fetuses from outbred CF1 mice, but other strains can be used as an alternative. Tissue culture dishes are preferably treated with 0.1% gelatin (type I). However, as demonstrated by the data disclosed herein, the ES-like cells of the present invention do not require feeder cells for most applications.

Serum-free progenitor media formulations for the maintenance and/or propagation of ES-like cells prepared according to the methods of the present invention are similarly well known in the art. The skilled artisan will readily appreciate that such progenitor media formulations are capable of supporting a wide variety of ES-like cells made following the methods of the present disclosure. Such formulations comprise N-(2-OH-ethyl-)piperazine-N′-(2-ethanesulfonic acid) at a concentration of 14-22 mM, sodium chloride at a concentration of 100-120 mM, histidine at a concentration of 0.1-0.25 mM, isoleucine at a concentration of 0.05-0.5 mM, methionine at a concentration of 0.1-0.5 mM, phenylalanine at a concentration of 0.1-0.5 mM, tryptophan at a concentration of 0.05-0.5 mM, tyrosine at a concentration of 0.1-0.5 mM and growth factors at a concentration of 0.3-30 ng/ml.

ES-like cells of the present invention, when propagated and expanded in progenitor media described herein and such media as are well-known in the art, are capable of novel limitless proliferation, without demonstrating any of the phenotypic or molecular characteristics of oncogenic transformed cells. That is, reprogrammed cells thirty days after infection, accelerate through the G1 phase, and reside primarily in the G2/M phase and, to a lesser extent, in the S phase of the cell cycle, but are not different sizes, strongly indicating a non-transformed phenotype. Further, in sharp contrast to cells infected with oncogenic viruses, reprogrammed cells of the present invention are mainly in the G2/M phase of the cell cycle, whereas cells transformed with oncogenic viruses are in mainly in the G1 phase. Further, the total cell count of M. leprae infected Schwann cells, when compared to control cultures, was significantly increased after 30 days in culture, further indicating proliferation without transformation, a characteristic feature of stem cells. Other stem cell characteristics include, but are not limited to, unusual cell cycle distribution, for example low percentage of G1 population and high percentage of S phase-ES cells reentering the S-phase of the cell cycle very shortly after exit from mitosis.

Another important aspect of G1 phase of control lies in the regulation of cyclin D1 expression by the RAS>MEK>ERK pathway. In ES-cells, inhibition of ERK phosphorylation by MEK inhibitors does not inhibit background expression of cyclin D1 (see Jirmanova et al., 2002, Oncogene 21:5515-5528). Similarly, ES-like reprogrammed cells of the invention also do not inhibit cyclin D1 in the presence of MEK inhibitors. This further demonstrates the striking similarity of the induction of ERK phosphorylation and cyclin D1 expression in a reprogrammed ES-like cell and an ES-cell as previously described.

In addition, despite sustained cyclin D and cyclin D1/cdk activity in the ES-like cells of the present invention, the cells do not exhibit a transformed phenotype in a standard plaque assay. That is, when compared to a well characterized transformed cell line, the ES-like cells of the present invention do not exhibit any characteristics of a transformed phenotype.

Despite continuous proliferation and expression of cyclin D, ES-like cells still express tumor suppressors such as H-cadherin, Wnt-5a, and caveolin-1, demonstrating that the cells of the present invention are not tumor cells, but rather ES-like cells.

The present invention also includes a method of identifying a compound that affects the cell cycle. The method comprises contacting a cell with a test compound and comparing the progression through the cell cycle in the cell contacted with the compound with the progression through the cell cycle in an otherwise identical cell, which is not contacted with the compound. If the level of cell cycle progression (e.g., cells in G2 or S phase as opposed to G1 phase) is higher in the cell contacted with the compound compared to the level in the cell that was not contacted with the compound, then that is an indication that the test compound affects the cell cycle.

The skilled artisan will further appreciate that the present invention includes a method of identifying a useful compound in a cell or an animal that mediates reprogramming of a cell. That is, the present invention includes methods of identifying a useful compound in a cell-free system. A cell-free system, as used herein, refers to an in vitro assay wherein the components necessary for a reaction to take place are present, but are not associated with a cell. Such components can include cellular enzymes, transcription factors, proteins, nucleic acids, and the like, provided that they are substantially free from a cell. As disclosed by the data herein, cell proliferation and reprogramming assays can be performed free of a cell or animal, including the use of immunoprecipitation assays, Western blots, gene arrays, and the like. Thereby, the present invention includes a method of identifying a useful compound for reprogramming cells or increasing cellular proliferation in a cell-free system.

The skilled artisan, when equipped with the present invention and the methods detailed herein, will readily be able to identify M. leprae proteins, glycoproteins, carbohydrates, saccharides, glycolipids, genes, and other components useful in reprogramming differentiated cells to a ES-like cell phenotype. Once an M. leprae component is identified, the component can be used as a pharmaceutical composition in a pharmaceutically acceptable carrier for the ex vivo or in vivo treatment of a disease or condition where reprogramming cells would be useful. Such diseases and conditions include, but are not limited to, Alzheimer's disease, multiple sclerosis, burn injuries, melanoma, other skin diseases where it is beneficial to replace skin with autologous or allogeneic skin transplants, diabetes, spinal cord injuries, other CNS injuries, including healthy tissue removed after tumor removal surgery and traumas, Parkinson's disease, and the like.

Pharmaceutical compositions comprising the useful component or cell (e.g., reprogrammed, converted, and re- or trans-differentiated cell) of the invention can be used either for the methods of the invention. That is, the pharmaceutical compositions comprising an M. leprae reprogramming factor and/or cells produced according to the methods disclosed herein can be used to de-differentiate/reprogram cells from a cell line, primary cells, or cells taken directly from a patient in need. Further, the M. leprae de-differentiation/reprogrammed cells can be administered directly to an animal, preferably a human, in order to treat or ameliorate a disease, disorder or condition.

As used herein, the term “pharmaceutically-acceptable carrier” means a chemical composition with which an appropriate M. leprae reprogramming factor may be combined and which, following the combination, can be used to administer the appropriate M. leprae reprogramming factor to a mammal or cells derived therefrom.

The pharmaceutical compositions useful for practicing the invention may be administered to deliver a dose of between 1 ng/kg/day and 100 mg/kg/day.

Pharmaceutical compositions that are useful in the methods of the invention may be administered systemically in oral solid formulations, ophthalmic, suppository, aerosol, topical or other similar formulations. In addition to the appropriate M. leprae reprogramming factor, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate M. leprae reprogramming factor according to the methods of the invention.

Compounds which are identified using any of the methods described herein may be formulated and administered to a mammal for treatment or alleviation of the diseases disclosed herein are now described.

The invention encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to an animal, preferably a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

VI. Kits

The present invention further comprises kits for preparing the methods of the invention. That is the kit can be used for, among other things, producing a reprogramming a cell and generating ES-like and stem cell-like cells, controlling the cell cycle, reprogramming a cell, and controlling cell proliferation, and the like. The kit comprises an M. leprae derived factors for reprogramming cells, controlling the cell cycle, and/or controlling cell proliferation, identified by the methods disclosed herein above, and an instructional material which describes the use of such components according to the methods disclosed elsewhere herein.

As used herein, an “instructional material” includes a publication, a recording, a diagram, or any other medium of expression which can be used to communicate the usefulness of the M. leprae derived factor of the invention in the kit for, among other things, effecting the reprogramming of a differentiated cell. Optionally, or alternately, the instructional material may describe one or more methods of re- and/or trans-differentiating the cells of the invention. The instructional material of the kit of the invention may, for example, be affixed to a container which contains the M. leprae, or component thereof, of the invention, or be shipped together with a container which contains the M. leprae, or component thereof. Alternatively, the instructional material may be shipped separately from the container with the intention that the instructional material and the M. leprae, or component thereof, be used cooperatively by the recipient.

The invention is now described with reference to the following Examples. These Examples are provided for the purposes of illustration only and the invention should in no way be construed as being limited to these Examples, but rather should be construed to encompass any and all variations which become evident as a result of the teaching provided herein.

### EXAMPLES

### Example 1

### Establishment of Human Schwann Cell Primary Cultures

M. leprae is a human pathogen, but a human model system does not exist to study the questions and issues related to the effects of M. leprae infection in human cells. Such a system would be valuable not only for development of therapeutic avenues, but also as a system to study the effect of M. leprae on the cell cycle, gene regulation, and the ability of M. leprae to reprogram various human cell types. This novel discovery, that M. leprae, or component thereof, can reprogram an adult somatic cell to an ES-like cell, which cell can then be differentiated to produce a cell of a desired tissue type different than, or the same as, the original cell, provides a powerful tool for cell-based therapies, among other things. The data disclosed herein demonstrate production, for the first time a highly purified human Schwann cell primary culture, established from different organ donors, as an ex vivo human model for M. leprae reprogramming, as well as for assessing other effects of M. leprae infection on human cells.

Human Schwann cells were isolated from peripheral nerve tissues obtained from the University of Miami Organ Procurement Organization (Miami, Fla.) following the harvest of organs for transplantation. Tissues consisting of nerve roots making up the Cauda equina were harvested within 2 hours of aortic clamping and stored at 4° C. in Belzer's (University of Wisconsin) Cold Storage Medium (DuPont, Inc., Wilmington, Del.) until processing, usually within 24 hours of harvest. Cauda equina roots were teased apart, minced into fragments and incubated overnight at 37° C. and 6% CO₂ in Dulbecco's Modified Eagle Medium (DMEM; GIBCO/InVitrogen, Grand Island, N.Y.) containing heat-inactivated 10% fetal bovine serum (FBS; Hyclone, Logan, Utah), 0.5 mg/ml collagenase type I (Worthington Biochemical Corp., Lakewood, N.J.) and 1 mg/ml dispase II (Roche Molecular Biochemicals, Indianapolis, Ind.). The resulting cell suspensions were rinsed 3 times with Leibovitz's L15 medium, and plated onto uncoated tissue culture-treated dishes in DMEM+10% FBS for 2 days at 37° C. and 6% CO₂. This enabled most endothelial cells, fibroblasts and macrophages to adhere, while leaving the Schwann cells in suspension.

The Schwann cells were then plated onto laminin-coated dishes (GIBCO/InVitrogen; 33 g/100 mm dish) in Medium A, consisting of DMEM, 10% FBS+2 nM forskolin (Sigma, St. Louis, Mo.) and 10 nM recombinant heregulin peptide 177-244 (Genentech, South San Francisco, Calif.) at a density of 500,000 cells/100 mm dish. Confluent cells were re-suspended by rinsing in Ca⁺⁺ and Mg⁺⁺-free Hank's Balanced Salt Solution (CMFHBSS) and brief (5 minutes at room temperature) exposure to a solution of 0.05% trypsin and 0.02% EDTA in CMFHBSS. Re-suspended cells were rinsed in L-15/10% FBS and re-plated in Medium A into new laminin coated dishes at a density of 500,000 cells/100 mm dish. The cultures were again grown to confluence.

Schwann cells harvested from such cultures were designated Passage 1 or P1. P1 cells were further purified by FACSVantage dual-laser flow cytometer (Becton-Dickinson, San Jose, Calif.) using p75 MAb as a specific marker for non-myelinating Schwann cells (Morrissey et al., 1995, J. Neurobiol. 28: 190-201). Highly purified Schwann cell population were obtained from each human donor using this protocol.

Schwann cells were seeded on collagen-coated flasks (BD BioScience, Bedford, Mass.) and propagated in larger scales. Schwann cells used in the experiments described herein were harvested at P3-P4. The purity of the final Schwann cell preparation prior to infection was about 98-100% as determined by p75 and S100 immunoreactivity. All Schwann cell preparations were highly effective for invasion by M. leprae regardless of donor variation.

M. Leprae and Infection of Primary Human Schwann Cells

Viable M. leprae used in this study was derived from the footpads of athymic nu/nu mice essentially is described in Truman and Krahenbuhl, (2001, Int. J. Lepr. Other Mycobact. Dis. 69: 674-681). Preparation and characterization of irradiated M. leprae was performed as described previously (Adams et al., 2000, Int. J. Lepr. Other Mycobact. Dis. 6: 1-10). Freshly harvested, viable or irradiated Thai-53 isolate of M. leprae were obtained from Dr. J. L. Krahenbuhl (National Hansen's Disease Programs Laboratory, Baton Rouge, La.). M. leprae infection was carried out by inoculation of 1×10⁸ bacteria in semi-confluent purified human primary Schwann cells (passage # 3) grown on collagen-coated flasks (BD BioScience). Schwann cells from at least 5 different human donors were used for infection.

M. leprae infection was performed essentially as described in Rambukkana et al. (2002, Science 296: 927-931), except human Schwann cells were used instead of rat Schwann cell-neuron co-cultures, and infected human Schwann cells were maintained at 33° C. in humidified CO² incubator. Briefly, 1 hour after incubation with M. leprae, excess bacteria were removed and the culture media was replaced with fresh Schwann cell media. Intracellular location of M. leprae was monitored after 24 and 48 hours using immunofluorescence using monoclonal antibody specific for M. leprae PGL-1 (Ng et al., 2000, Cell 103: 511-524) and also using electron microscopy. Infected cultures were fed with Schwann cell media every 2 days and the cultures were maintained up to about 30-40 days.

The Results of the experiments disclosed herein are as follows.

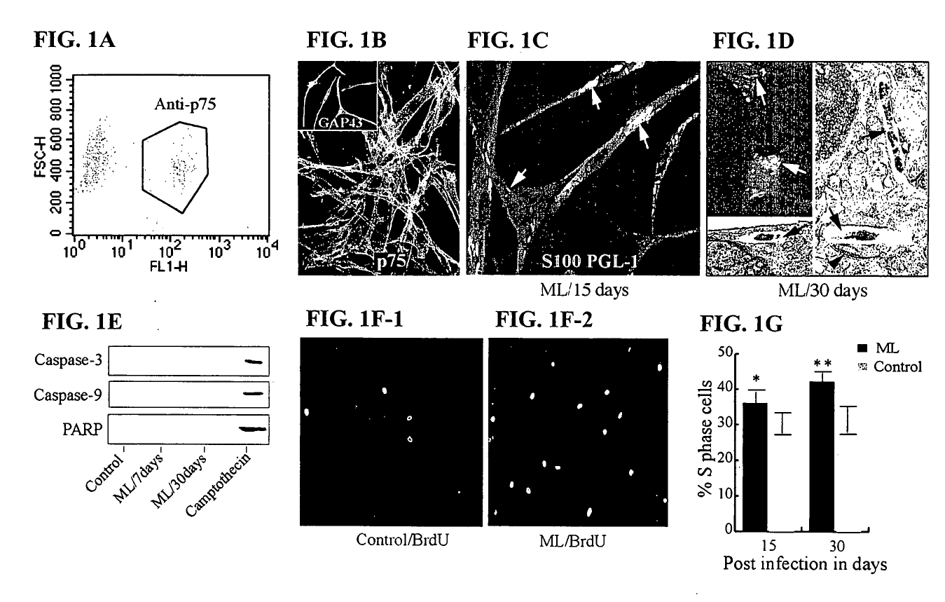

Primary Schwann cell cultures phenotypically and functionally resemble non-myelinated Schwann cells in vivo. They comprise all surface and intracellular Schwann cell markers, and react to axonal contact in the same way as non-myelinated Schwann cells in vivo. However, in the ex vivo model presented here, these Schwann cells lack axons and the basal lamina (see, e.g., Ng et al., 2000, Cell 103:511-524). Importantly, the ex vivo Schwann cell model demonstrates similar M. leprae invasion efficacy as reported in human leprosy patients see e.g., Shetty et al., 1998, J. Neurol Sci 88:115-131. Upon inoculation, most M. leprae can be found intracellularly within 24 hours. Once intracellular, M. leprae maintains its own viability as well as that of the Schwann cells for long periods. Morphological analyses demonstrate that infected Schwann cells are indistinguishable from uninfected cells after 30 days in culture, and due to the slow doubling time of M. leprae, primary Schwann cells can be maintained in culture for several weeks. (FIGS. 1B, 1C and 1D). Further, the preferred growth temperature of M. leprae is 33° C. Primary Schwann cells maintained at 33° C. demonstrate no detectable differences in phenotypic or functional properties.

Not only do intracellular M. leprae maintain Schwann cell survival, but it also has the capacity to induce Schwann cell proliferation. BrdU uptake in infected Schwann cells was significantly higher (approximately 20%) than in uninfected cultures from day 3 to day 30, demonstrating the proliferative capacity of M. leprae-infected Schwann cells (FIGS. 1F and 1G). Further, infected Schwann cells showed no signs of apoptosis, even after 30 days, as demonstrated by a lack of caspase activity (FIG. 1E). Uninfected and infected Schwann cells do not express the apoptosis markers caspase 3, caspase 9, or PARP, while Schwann cells treated with the apoptosis stimulator camptothecin express all three apoptosis markers. Thus, the data disclosed herein demonstrate that intracellular M. leprae activate an effective survival program that maintains and induces proliferation of Schwann cell to the advantage of M. leprae survival.

### Example 2

### Functional Genomics of Human Schwann Cells Infected with M. leprae

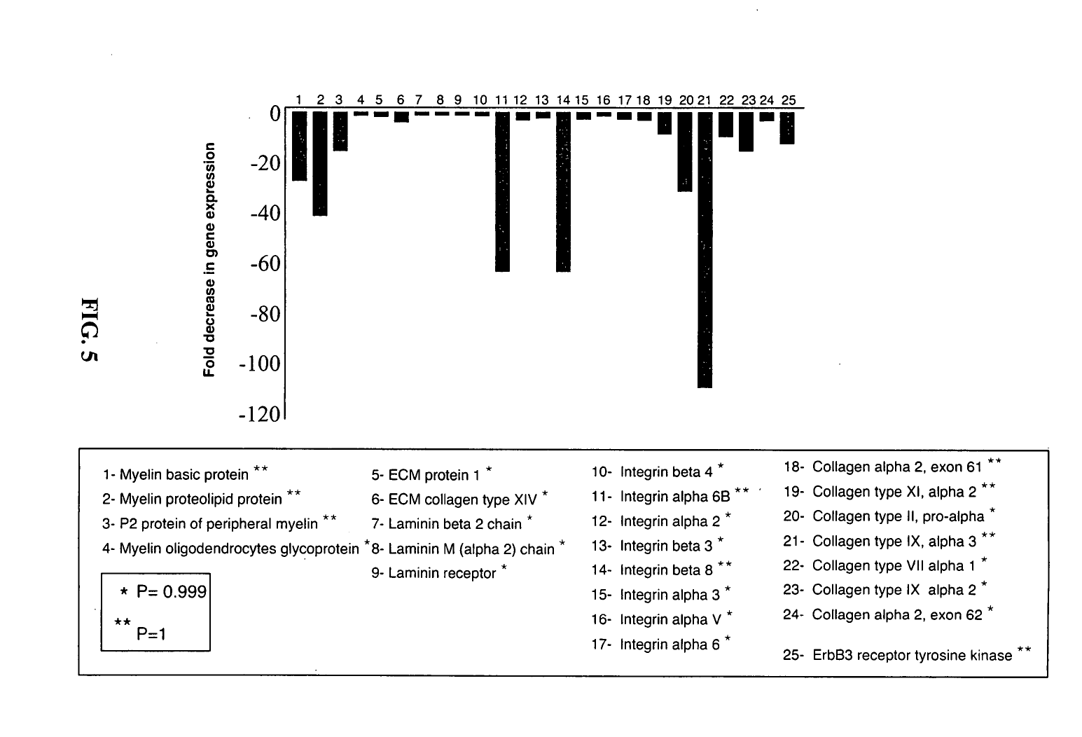

The data disclosed herein demonstrate that M. leprae is capable of maintaining its viability in Schwann cells despite massive gene decay and deletion in the M. leprae genome. Further, M. leprae promotes Schwann cell survival and proliferation during infection, without morphological, phenotypic, or functional changes in the cells. While not wishing to be bound by any particular theory, the data demonstrate that M. leprae simplifies the Schwann cell intracellular environment to facilitate its slow growth and propagation without interference from the differentiated cell's abilities to prevent such growth. For example, the formation of myelin sheath, a typical example of differentiation, within Schwann cell cytoplasm restricts the intracellular space for bacterial replication and growth inside Schwann cell. M. leprae has the ability to down-regulate all the genes necessary for myelin synthesis, such as genes encoding for major ECM proteins and their receptors as well as Schwann cell lineage-specific marker ErbB3/neuregulin receptor (FIG. 5). Thus, while M. leprae is altering the cell for its own benefit, it is regulating the expression of genes that when expressed, reprogram the cell into an ES-like phenotype characterized by alternating upregulation of developmental, embryonic, and proliferation-related genes while down-regulating differentiation and apoptotic defense genes. The data disclosed herein demonstrate that M. leprae controls gene expression in human cells without causing any detectable damage to cells, especially genes involved in metabolism and the respiratory chain, cell cycle regulation, growth factor and their receptor up-regulation, and transcription factor expression. Further, no cytokine genes and very few apoptotic genes are upregulated, indicating that M. leprae encourages the survival and proliferation of human Schwann cells for its own benefit.

Genearray Analysis

Human genome AFFYMETRIX GENECHIPS were used to determine differential cell cycle gene expression of human primary Schwann cells in response to intracellular M. leprae. Total cellular RNA was isolated from M. leprae-infected and non-infected-Schwann cells at days 3, 7, 15, and 30 using RNeasy spin columns (Qiagen, Valencia, Calif.). Briefly, the total RNA (5 μg) was reverse-transcribed to cDNA and double-stranded cDNA synthesis was performed as described in the Expression Analysis Technical Manual (AFFYMETRIX, Santa Clara, Calif.). The cRNA reactions were performed using the BioArray High-Yield transcript labeling kit (Enzo Diagnostic Inc., Farmingdale, N.Y.). 15 μg of labeled cRNA was fragmented for 35 minutes at 94° C. using fragmentation buffer (200 mM Tris-acetate, pH 8.1, 500 mM Potassium acetate, 150 mM Magnesium acetate).

AFFYMETRIX Test3 arrays and human genome U95Av2 arrays were probed, hybridized, stained and washed according to the manufacturer's instructions. Expression analysis of the differentially expressed genes was performed with the AFFYMETRIX Microarray Suite 5.0 software to obtain absence/presence calls, fold differences and significance (p values). Differentially expressed genes of the M. leprae infected Schwann cells were then clustered according to functional annotations using the Data Mining Tool 3.0 software (AFFYMETRIX) by searching the GeneBank, UniGene and LocusLink databases through the NETAFFX portal (AFFYMETRIX).

The Results of the experiments disclosed herein are as follows.

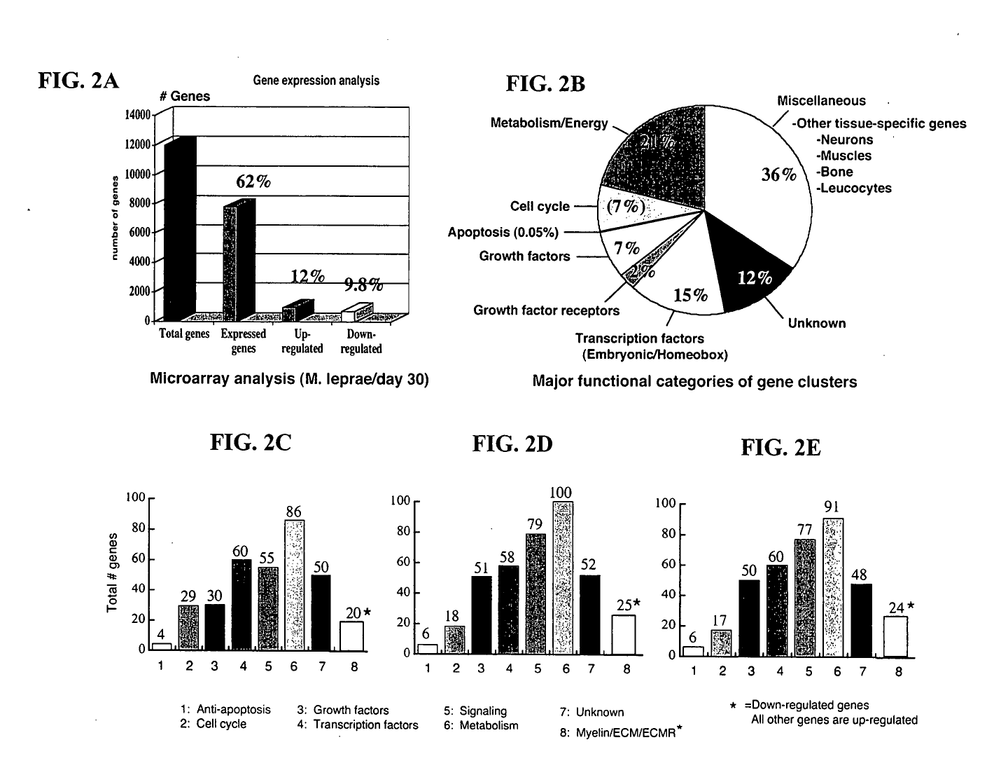

Of the 12,000 distinct human genes on the chip array, 7,440 (62%) were expressed in primary human Schwann cells, and 1,621 (22%) were differentially expressed in response to intracellular M. leprae. Of these genes, 892 (12%) were upregulated and 729 (9.8%) were down-regulated (FIG. 2). According to database annotation, the expressed genes were categorized according to known functions.

The Schwann cell response to intracellular M. leprae was dominated by genes encoding key enzymes involved in metabolism and the respiratory chain (86 genes), cell cycle regulators (29 genes), growth factors (30 genes), and their receptors (10 genes), and transcription factors (60 genes). No cytokine genes and only 3 apoptotic genes were altered by M. leprae. There were 50 unknown genes and 140 miscellaneous genes including genes for matrix proteins and their receptors, cytoskeleton proteins, phosphatases, and RNA transport proteins (FIG. 2).

Moreover, the data disclosed herein demonstrate that not only viable M. leprae, but irradiated M. leprae, comprising cell wall components and fragments thereof, are capable of altering the expression of major functional gene clusters in human Schwann cells. Two experiments comprising infecting primary Schwann cell cultures with viable M. leprae and one experiment comprising infecting primary Schwann cells with irradiated M. leprae resulted in similar results. Specifically, in all three experiments, anti-apoptosis genes, cell cycle genes, growth factor genes, transcription factor genes, signaling genes, metabolism genes, and unknown genes were all upregulated, while myelin/ECM/ECMR (i.e., Schwann cell differentiation markers) were consistently down-regulated (FIGS. 2C-D).

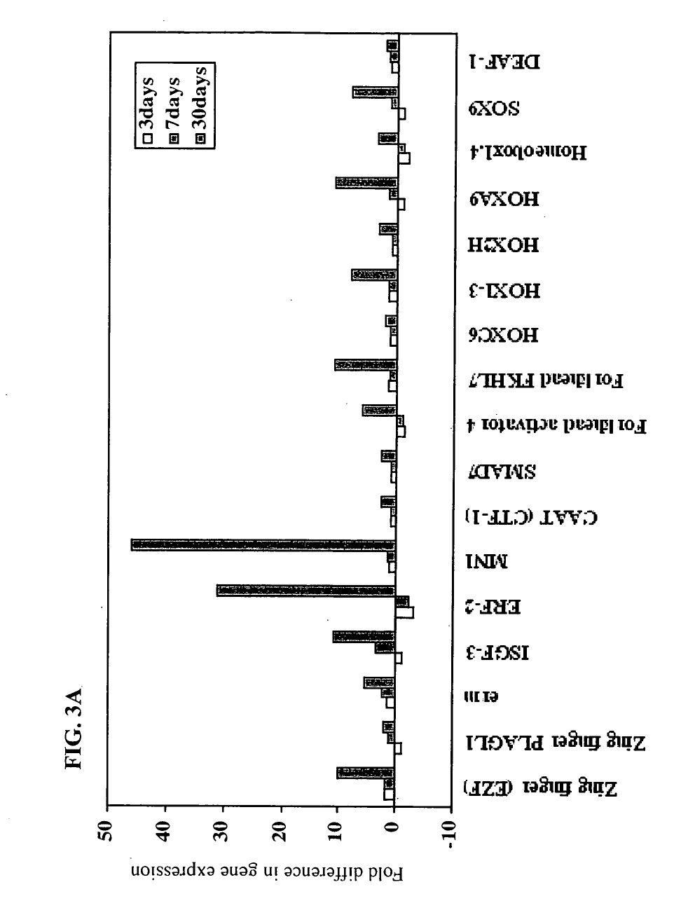

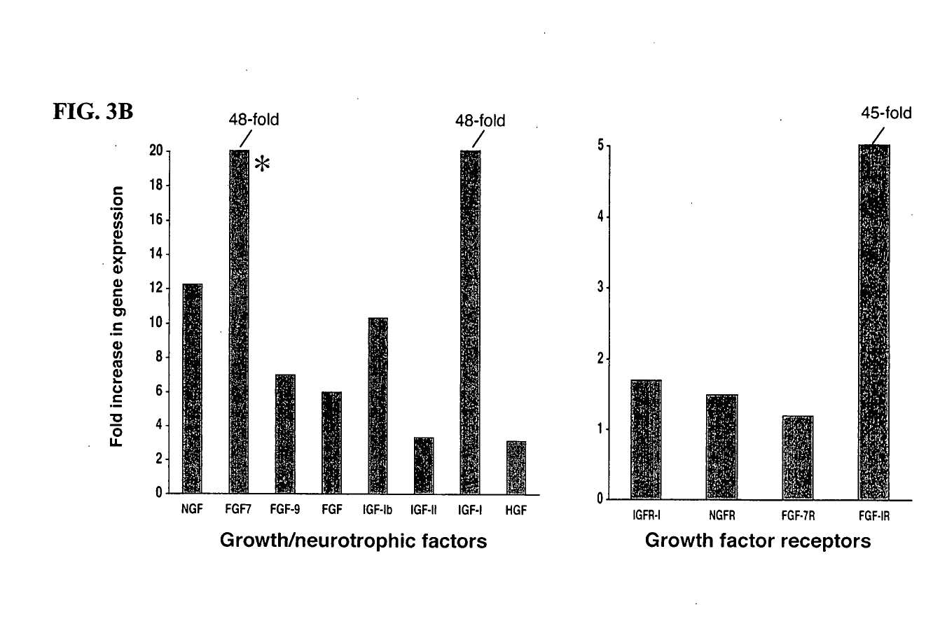

The transcription related genes affected by intracellular M. leprae include the transcription factors EZF (beta-globin promoter activator), PLAGL1 (tumor suppressor), erm (expressed in multipotent neural crest cells), ISGF-3 (tamoxifen-induced tumor repressor), ERF-2 (transcription factor with unknown function), MN1 (transcription factor with unknown function), CTF-1 (chromatin regulation in response to extracellular factors), SMAD7 (negative feedback control for TGF-beta induced apoptosis), Forkhead activator 4 (p53 and WT-1 activated transcription factor), Forkhead FKHL7 (activated during late blastula, early gastrula phases of embryogenesis), HOXC6 (transcription activator or repressor), HOX 1-3 (embryonic regulation genes), HOX2H (embryonic transcription regulation), HOXA9 (embryonic transcription regulation), Homeobox 1.4 (embryonic transcription regulation), SOX9 (express in CNS stem cells, gonadal and osteoblast differentiation) and DEAF-1 (general early embryonic development). The data therefore disclose that the intracellular presence of M. leprae resulted in the increased expression of multiple transcription factors after about 30 days, but not at 3 or 7 days post-infection (FIGS. 3A and 4). Further, the transcription factors upregulated are those that are often involved in embryogenesis, ES cell development and regulation. It should also be noted that these transcription factors are never been described in normal adult Schwann cells.

The growth/neurotrophic factors and growth factor receptors upregulated by intracellular M. leprae include Nerve Growth Factor (NGF; promotes survival and nerve process outgrowth of specific classes of neurons), Fibroblast Growth Factor-7 (FGF-7; mesenchymal stimulation of epithelial cell growth), Fibroblast Growth Factor-9 (FGF-9; physiological role is unknown), FGF/bFGF (stimulates stem cell proliferation and somatic cellular proliferation, inhibits differentiation of many cell types, embryonic development), IGF-Ib (cell survival, cell metabolism promoter, cell proliferation), IGF-II (promotes cell survival and metabolism, promotes cell proliferation), IGF-I (promotes cell survival and metabolism, promotes cell proliferation), and HGF (potent hepatocyte growth factor and proliferation inducer). Further, intracellular M. leprae, or components thereof, upregulate the expression of most of these growth factor receptors in human Schwann cells. Such receptors include, IGFR-I (insulin growth factor receptor-1), NGFR (nerve growth factor receptor), FGF-7R (fibroblast growth factor-7 receptor), and FGF-IR (fibroblast growth factor-I receptor) (FIG. 3B).

Thirty days after M. leprae infection, Schwann cells expressed a number of developmental and embryonic genes not usually expressed in differentiated cells. These developmental and embryonic genes include IFN-RD1, BMP-11, -4, -2A, -2B, -3b (regulate cell division, apoptosis (programmed cell death), and cell migration,), Sox 9, Hox 1.4, Hox MEIS2, PMX-1/PHOX-1, HOX 1.3, Hox MHB24, ZIC, SIX1, HOXC5, HOXA9, and HOX2H (which are crucial embryonic morphogenesis and developmental genes expressed in a wide variety of phylum) (FIG. 4). Thus, the data demonstrate that intracellular M. leprae induces the expression of critical genes present during the embryonic and early developmental phases of cell and organism differentiation.

Also, the presence of intracellular M. leprae down-regulates Schwann cell myelin genes, thus ameliorating the differentiated phenotype of quiescent human cells. Intracellular M. leprae significantly down-regulates the following Schwann cell myelin and differentiation associated genes characteristic (or associated with) a differentiated cell state: myelin basic protein, myelin proteolipid protein, P2 protein of peripheral myelin, myelin oligodendrocytes glycoprotein, ECM protein 1, ECM collagenase type XIV, laminin beta 2 chain, laminin M (alpha 2) chain, laminin receptor, integrin beta 4, integrin alpha 6b, integrin alpha 2, integrin beta 3, integrin beta 8, integrin alpha 3, integrin alpha V, integrin alpha 6, collagenase alpha 2, exon 61, collagen type XI alpha 2, collagen type 2 pro-alpha, collagen type IX alpha 3, collagen type VII alpha 1, collagen type IX alpha 2, and collagen alpha 2 exon 62 (FIG. 5).

ErbB3, a neuregulin receptor, is extremely important during the development of Schwann cells and for the maintenance of the Schwann cell lineage. Homozygous ErbB3 knockout mice embryos lack Schwann cell precursors and do not develop mature Schwann cells that accompany peripheral axons and motor neurons. Further, and peripheral axons or motor neurons that form in the knock-out mice undergo cell death due to the lack of Schwann cells (Riethmacher et al., 1997, Nature 389: 725-730). Thus, ErbB3 is a crucial element in Schwann cell development, identity, formation, and function.

In order to further demonstrate that M. leprae infected cells lose differentiated markers associated/characteristic of a differentiated cell state, (protein and functional level), human Schwann cell and rat dorsal root ganglion (DRG) cell cultures were analyzed for ErbB3 expression using the Western blotting methods detailed below. Briefly, human Schwann cells and rat DRG cultures expressed ErbB3 after 30 and 25 days, respectively, of culture. However, when the same cells were infected with M. leprae the same amount of time, ErbB3 protein was not detected (FIGS. 6A and 6B). Therefore, M. leprae inhibited expression of the crucial Schwann cell receptor ErbB3. Intracellular M. leprae also inhibited ErbB3 expression in rat DRG cells. Further, ErbB3 transcripts were down regulated by 20-fold in M. leprae infected cells compared to non-infected Schwann cells.

Similarly, rat explant DRG cultures lost the ability to form compact myelin sheaths when they were infected with M. leprae for 25 days. Both electron microscopy and antibodies to myelin basic protein (MBP) failed to detect compact myelin formation in M. leprae infected cells while uninfected cells formed compact myelin with visible nodes of Ranvier (FIG. 6C-6F). In addition, rat DRG cultures demonstrated a significant down regulation of P0 protein (P0 and with MBP are the two major proteins in compact myelin sheaths in peripheral nerves) in immunoblots from infected and uninfected rat DRG cultures (FIG. 6G). Thus, not only does M. leprae upregulate embryonic, transcription, and growth factor genes in Schwann cells but, genes characteristic of the differentiated cell phenotype (i.e., PO and MBP) are significantly down-regulated.

### Example 3

### M. leprae Infected Human Schwann Cells Proliferate Continuously But do not Undergo Transformation

The hallmark characteristics of stem cells are: i) indefinite proliferation in vitro in an undifferentiated state; (ii) a normal karyotype through prolonged culture; and (iii) the potential to differentiate into other cell types. Stem cells maintain the ability to proliferate in vitro without evidence of transformation, which is in contrast to other proliferative cells transformed with oncogenes or oncoviruses, or derived from tumor lines, but without telltale signs such as lack of contact inhibition. The data disclosed herein demonstrates, for the first time, a method to reprogram differentiated cells into an ES-like cell without transformation. That is, the data demonstrate that adult cells can be reprogrammed and display many, if not all of the traits of a stem cell, including continuous proliferation without transformation.

Cell Lines

The breast adenocarcinoma epithelial cell line SKBR-3 and fibroblast cell line NIHJ3T3 were obtained from ATCC (Manassas, Va.) and propagated according the culture protocols from ATCC. SV40-transformed human Schwann cell line used herein has been described previously (Rambukkana et al., 1998, Science 282:2076-2079).

Antibodies

Primary rabbit polyclonal antibodies used in this study included anti-phospho-Erk1/2 (Thr202/Tyr204), anti-phospho-GSK-3β, anti-phospho-β-catenin, anti-phospho-Akt, anti-phosphopanPKC, anti-phospho-p38, anti-phospho-JNK, anti-phospho-Rb1 and anti-phospho-Elk (Cell Signaling Technology, Beverly, Mass.); anti-human Cyclin D1, anti-human p21, anti-human caveolin-1 (Santa Cruz Biotechnology, Santa Cruz, Calif.), and anti-S100 (Dako, Carpinton, Calif.). Primary mouse monoclonal antibodies used were anti-human Cyclin B, anti-human cyclin A, anti-human Rb1, anti-human Rb2, anti-human Cdk4 (BD Biosciences, Lexington, Ky.); anti-BrdU (Roche Molecular Biochemicals, Indianapolis, Ind.); anti-p75 (Dr. P. M. Wood, University of Miami, Fla.); anti β-Actin, anti-human vinculin, anti-FLAG (Sigma Chemical Co., St. Louis, Mo.), and anti-M. leprae PGL-1 (Dr. A. Kolk, Royal Tropical Institute, Amsterdam).

Immunofluorescence

Infected and control Schwann cells were trypsinized and seeded onto collagen-coated coverslips (BD Biosciences, Bedford, Mass.) and incubated for a desired time until they firmly adhered. The cells were washed twice with phosphate buffered saline (PBS) and fixed with cold methanol for 10 minutes. After blocking and incubation with the relevant primary antibodies, the cells were washed and incubated with affinity purified, rhodamine-conjugated goat anti-rabbit IgG, and/or fluorescein-conjugated goat anti-mouse IgG (Chemicon International, Temecula, Calif.). Images were captured using a Zeiss LSM 510 confocal microscope.

Transformation Assay

Transformation assays were performed according to an established protocol (Lee et al., 2000, Mol. Cell. Biol. 20: 672-683). Briefly, M. leprae-infected and non-infected-Schwann cells and SK-BR-3 breast cancer cells were trypsinized and seeded onto 100-mm-diameter Poly-L-Lysine-coated dishes. The culture media were changed twice weekly for 21 days. Cells were washed twice with PBS, fixed with 10% acetic acid for 10 minutes and then stained with 0.4% crystal violet (Sigma) in 10% ethanol for 10 minutes. The dishes were washed, inverted and dried at room temperature. Plaque formation was evaluated by colony counting under an inverted microscope.

Cell Cycle FACS Analysis

Human primary Schwann cells, that had been cultured for 15 and 30 days with and without M. leprae were trypsinized and adjusted to equal cell number. For the experiments with kinase inhibitors, synchronized infected and control cells were treated with pre-evaluated concentrations of various inhibitors as described previously elsewhere herein, and with different combinations of inhibitors before being processed for cell cycle FACS (Lemoine and Marriott, 2001, J. Biol. Chem. 276:31851-31857). To assess the effect of Lck inhibitor on heregulin-induced cell cycle progression, synchronized Schwann cells were treated with heregulin for 30 minutes in the presence and absence of Lck inhibitor. Cells were then washed with PBS, fixed with 85% ethanol overnight at 40° C. and suspended in 1 ml PBS containing 1% FBS, RNase A (250 μg/ml; Roche Molecular Biochemicals) and 100 μg/ml Propidium Iodide (Sigma) for 30 minutes at 37° C. Stained cells were washed twice with PBS and subjected to FACS analysis on a Becton Dickinson FACScan cytometer (Ruhl et al., 1999, J. Biol. Chem. 274: 34361-34368). The cell cycle profile was analyzed with CellQuest software (Becton & Dickinson).

Western Blot

Total protein extracts were prepared from M. leprae infected and non-infected human Schwann cells in cell lysis and nuclear extraction buffer containing 62 mM Tris-HCl pH 6.8, 2% SDS, 10% Glycerol, 50 mM DTT and 0.2% protease inhibitors cocktail (Sigma, St. Louis, Mo.). Lysates were cleared by centrifugation and the protein content was determined according to the BCA method per manufacturer's instructions (PIERCE Chemical, Rockford Ill.). 40 μg of total cellular extracts were separated by sodium dodecyl sulfate-polyacrylamide gel electrophoresis (SDS-PAGE) and transferred to nitrocellulose membranes (Schleicher & Schuell, Keene, N.H.). Bound antibodies were detected using species-specific secondary antibodies coupled to HRP (Cell Signaling Technology, Beverly, Mass.) followed by enhanced chemiluminescence development (PIERCE Chemical, Rockford Ill.).

Protein expression intensity was normalized using β-actin expression as an internal control marker and quantified with a densitometer (Alpha Innotech Corporation, San Leandro, Calif.) using the Alpha Imager 2200 v.5.5 software. For preparation of nuclear and cytoplasmic extracts, cells were incubated in a buffer containing 10 mM HEPES pH 7.5, 60 mM KCl, 1 mM EDTA, 0.1% NP-40, 1 mM DTT, 1 mM PMSF and protease inhibitors cocktail for 10 minutes on ice. Lysates were then centrifuged for 10 minutes and the supernatant, which comprised of cytoplasmic extract, was collected. The pellet was re-suspended in a buffer containing 50 mM Tris-HCl pH 7.5, 150 mM NaCl, 1% sodium lauryl sarcosine, 1% NP-40, 0.1% SDS, 1 mM EDTA and protease inhibitors cocktail. After brief sonication, the lysates were centrifuged for 10 minutes and the supernatant, consisting of nuclear extract, was collected. The protein content in both extracts was determined using the BCA method per manufacturer's instructions (Pierce Chemical, Rockford, Ill.).

The Results of the experiments disclosed herein are as follows.

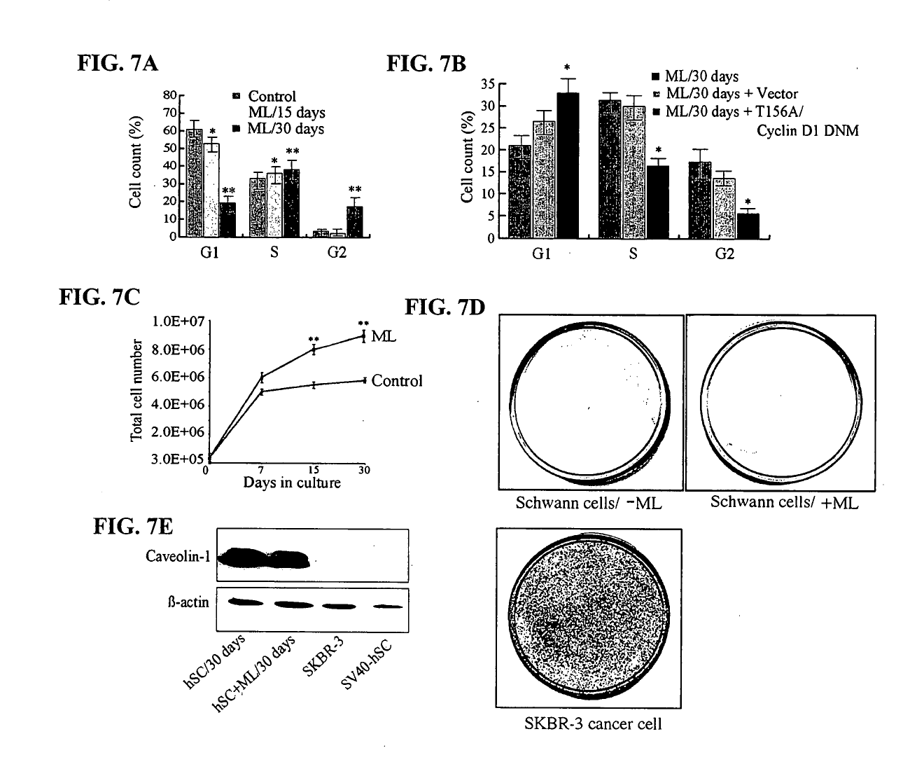

Cell cycle FACS analysis of asynchronous Schwann cell cultures 15 and 30 days after infection were performed according to the methods described elsewhere herein. The cell cycle profile of propidium iodide-treated Schwann cells revealed that total cell counts in G1 phase of 30 day infected Schwann cells was significantly reduced as compared to controls whereas the percentage of infected cells in S phase and G2/M phase were dramatically increased when compared to otherwise identical uninfected cells (FIG. 7). These results indicate that intracellular M. leprae accelerated G1 phase progression with earlier S phase entry, and that significant numbers of cells entered the G2/M phase. However, the accelerated cell cycle progression and reduced time in G1 to S phase transition did not affect the size of infected cells, which were indistinguishable from uninfected cells. This is in contrast to cells infected with certain oncogenic viruses that reduce the size of the cells as they accelerate through the G1 phase, ultimately resulting in cellular transformation in primary cultures. Moreover, oncogenic virus-infected cells do not increase the G2/M phase population. In contrast, 30 days after M. leprae infection, primary Schwann cells demonstrated a markedly increased G2/M phase population. Schwann cells in the G2/M phase were dramatically increased in cultures infected for 30 days when compared with those infected for 15 days with uninfected controls cells. Thus, the present data demonstrate that bacterial regulation of the cell cycle is dramatically different than the regulation mediated by infection with oncogenic viruses. Further, the data demonstrate that intracellular M. leprae assemble key cell cycle regulators essential for the maintenance and completion of the normal cell cycle division.

In order to further examine the role of M. leprae infection on cell progression through the cell cycle, and its effect of increasing cellular proliferation, the total Schwann cell count during the course of the infection was assessed/determined/investigated. Equal numbers of Schwann cells were seeded and propagated with and without M. leprae under the experimental conditions described elsewhere herein. The total cell count of 15-day and 30 day infected cultures, but not 7 day cultures, were significantly increased (p<0.0001) compared to controls (FIG. 7). These data demonstrate the ability of intracellular M. leprae to increase proliferation of human cells, especially Schwann cells, during long-term infection most likely as a result of positive regulation of cell cycle progression.

To further analyze if proliferating Schwann cells undergo transformation, a standard plaque-forming assay (described previously elsewhere herein) was employed. This assay identified any detectable transformation in 30 day infected Schwann cells compared to control cells and compared with a well characterized transformed breast cancer cell line, SK-BR-3, used as a positive control. SK-BR-3 cells formed numerous plaques as expected, but plaque formation was not detected in 30-day infected Schwann cells, which were almost identical to non-infected Schwann cells. Infected Schwann cell plaque assays were indistinguishable for plaque assays of uninfected Schwann cells However, infected Schwann cell plaque assays were in sharp contrast to the plaque forming properties detected for SKBR-3 cells in the plaque assays (FIG. 7). Therefore, the data disclosed herein demonstrate that the non-plaque forming phenotype of infected Schwann cells, in contrast to the plaque forming phenotype caused by oncogenic viruses that induce transformation in primary cells while accelerating G1 phase progression and proliferation is more closely similar to non-transformed cells. Therefore, M. leprae infection induced a novel and tightly controlled cell cycle regulation, with increased proliferation, but without inducing transformation.

Transformed cells characteristically lack contact inhibition due to altered adhesive interactions and cytoskeletal organization (Pawlak and Helfman, 2001, Curr. Opin. Genet. Dev. 11: 41-7). Members of the cadherin family, which selectively mediate cell recognition and adhesion, are associated with neoplastic behavior (Takeichi, 1991, Science 251: 1451-1455). Loss of or reduced expression of several members of the cadherin family, including H-cadherin, has been described in epithelial tumorigenesis. It has also been hypothesized that H-cadherin acts as a tumor suppressor (Toyooka et al., 2001, Cancer Res. 62: 3382-3386). In order to determine whether genes encoding cadherins are expressed in M. leprae infected Schwann cells, the gene array methods detailed above were employed. The genearray data demonstrate that H-cadherin was significantly upregulated in 30-day infected primary Schwann cell cultures in four reproducible experiments. Further, increased detection of H-cadherin protein in lysates of infected Schwann cells further confirm the genearray experiments. Thus, the data demonstrate increased levels of H-cadherin mRNA as well as increased level of protein.

The genearray data further demonstrate that expression of a member of a non-transforming class of genes, i.e., the Wnt gene family, Wnt-5a, was upregulated in M. leprae infected Schwann cells. Loss of Wnt-5a expression is correlated with aggressive tumor phenotypes in ductal tumors, and direct inhibition of Wnt-5a causes mammary cell transformation, suggesting that Wnt-5a has a tumor suppressor activity rather than oncogenic activity. Thus, the data not only show the increased expression of H-cadherin but also increase expression of Wnt-5a in Schwann cells. This is the first instance where these two proteins have been upregulated in a cell and further demonstrate that upregulation of ES markers and upregulation of markers known to be associated with a non-transformed phenotype are mediated by M. leprae infection. These data further confirm the lack of transformation in a now continuously proliferating cell.

In addition, calveolin-1, a principal member of the caveolae membrane, is down-regulated in transformed cells and the loss of caveolin-1 protein expression is sufficient to mediate cellular transformation. The data disclosed herein demonstrated that the transformation suppressor calveolin-1 is present in both infected and non-infected human Schwann cells, but is greatly diminished in the transformed cancer cell line SKBR-3. The data disclosed herein demonstrate that there is no difference in caveolin-1 gene or protein expression in infected or non-infected Schwann cells, whereas the expression of caveolin-1 was significantly down-regulated in SV40-transformed human Schwann cell lines and SK-BR-3 cancer cells. These data further demonstrate molecular evidence that infected Schwann cells exhibit increase proliferation without concomitant acquisition of a transformed phenotype.

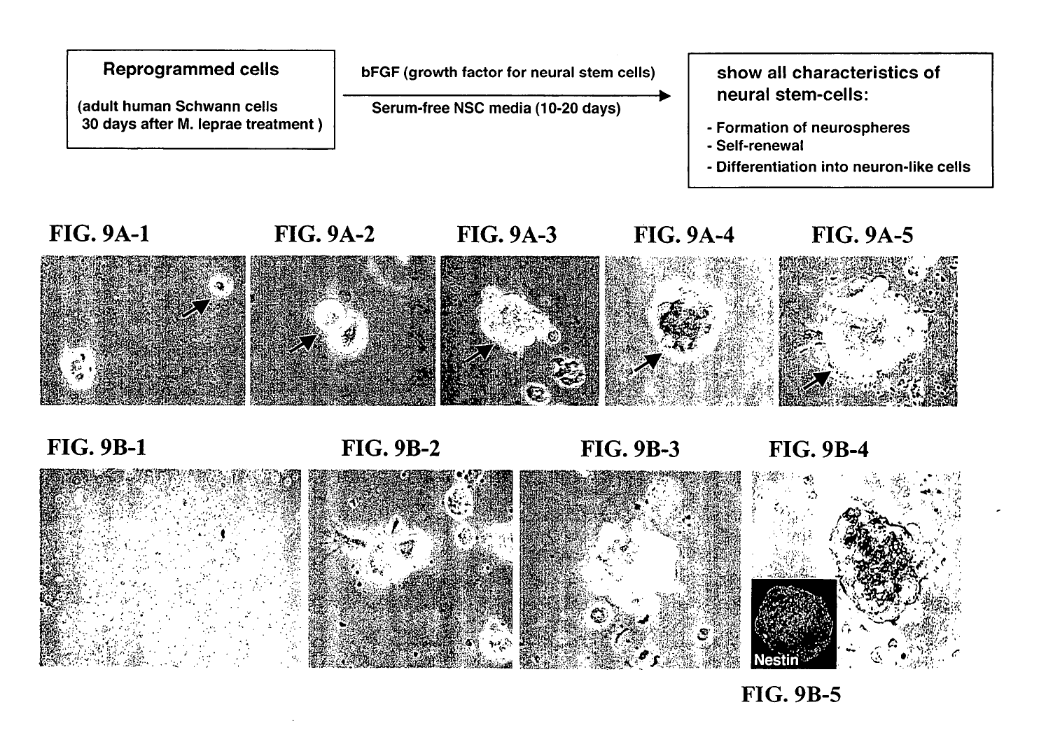

As further evidence that the present invention provides a method of generating ES-like cells that acquire the ability to proliferate continuously without concomitant development of a transformed phenotype, the structural components of M. leprae infected Schwann cells were analyzed. Transformation of normal cells with the ras oncogene disrupts stress fibers and focal contact organization, mainly due to constitutive activation of the Ras/MAPK pathway (Pawlak and Helfman, 2001, Curr. Opin. Genet. Dev. 11: 41-47). Such alterations in actin cytoskeletal structures are associated with decreased expression of numerous cytoskeletal proteins. Genearray analysis (described above) of 30-day infected and non-infected human Schwann cells showed no change in any mRNA transcripts encoding cytoskeletal proteins. Further, M. leprae infected cells maintained well-developed stress actin fibers and focal contacts as demonstrated by immunolabeling with phalloidin and anti-vinculin antibodies (FIG. 9). In contrast, the SK-BR-3 breast cancer cell line demonstrated the cytoskeletal disarray characteristic of transformed cell types, including significant disruption of vinculin and actin fiber formation.

In sum, the data disclosed herein clearly demonstrates that M. leprae infected cells are capable of significantly greater proliferation than uninfected cells, yet do not demonstrate any indication of transformation as measured by art-recognized assays for detection of transformation in a cell. This property is a hallmark of ES-like cells in vitro.

### Example 4

### Regulation of Cell Cycle by Intracellular M. leprae

As demonstrated by the data disclosed elsewhere, intracellular M. leprae can maintain its survival in infected cells by among other things, simplifying the Schwann cell intracellular environment, and down-regulation of differentiation process, as well as upregulating expression of transcription factor (i.e., embryonic and developmentally-related genes) and increasing infected cell proliferation in a highly controlled manner.

The data disclosed herein demonstrate for the first time that M. leprae induces Schwann cell proliferation by controlling cell cycle regulators. As disclosed previously elsewhere herein, intracellular M. leprae induced Schwann cells to accelerate through the G1 phase and a majority of infected cells were detected to be in the S and G2 phases of the cell cycle. Cyclin D1 is the major regulator involved in the G1 to S phase transition and in the proliferative phase of the cell cycle. These data suggest that M. leprae can affect activation, transcription, or another aspects of cyclin D1 activity. Further, the fact that a significant majority of M. leprae infected Schwann cells are in S and G2 phase indicates that cell cycle regulators, such as cyclin B, cdc2 (CDK1) and cdc25, may mediate the accelerated cell cycle progression of M. leprae infected Schwann cells. In addition, increased expression of key cell cycle molecules such as ubiquitins, RNA polymerase II, and MCM6 (component of DNA replication machinery), in infected cell, indicate that M. leprae can induce a novel pathway i.e., which do not involve the cyclin/cdk system for initiating cellular proliferation without transformation and controlling the cell cycle.

Electrophoretic Mobility Shift Assays (EMSA)

Biotinylated and unlabeled (cold) double-stranded oligonucleotides containing the consensus binding site of recognized by the transcription factor E2F-1 (5′-ATTTAAGTTTCGCGCCCTTTC TCAA-3′) (SEQ ID NO:1), were purchased from Panomics Inc. (Redwood City, Calif.). Nuclear extracts of Schwann cells were prepared as described above. Equal amounts (5 μg) of infected and control Schwann cell nuclear extracts were mixed with 2 ng of biotin-labeled probe in 10 μl of binding buffer containing 2 μg of poly(dI-dC) and incubated at 20° C. for 30 minutes. For competition experiments 10-fold of unlabeled (cold) oligonucleotide was included in the reaction mixture. Protein-DNA complexes were separated on nondenaturing 4% polyacrylamide gels and transferred to Biodyne B membranes (Pall, East Hills, N.Y.). Bound proteins were detected using. HRP-conjugated Streptavidin with enhanced chemiluminesence per manufacturer's instructions (Pierce Chem).

Real-Time Quantitative RT-PCR (TagMan) Analysis of Cell Cycle Genes

Primers and fluorescently labeled probes for detecting the coding regions of human cell cycle regulators, i.e., cyclin D1, Rb1, p57(Kip2), p21, p16, MCM6, RNA polymerase II, cyclin B, and Rb2 genes and GAPDH house keeping genes, were designed using Vector NTI software (Informax, Frederick, Md.). Total RNA was isolated from M. leprae-infected and non-infected Schwann cells at 7 and 30 days, and the RNA was reverse transcribed to cDNA using Superscript II reverse transcriptase per manufacturer's instructions (Invitrogen Life Technologies, Carlsbad, Calif.) and a mixture of oligo-d(T) and random hexamer primers (Invitrogen Life Technologies).

The cDNA produced was subsequently PCR amplified for 30 cycles using the various specific primers and fluorescent probes in a 384-well GeneAmp PCR system 9700 (Applied Biosystems, Foster City, Calif.). The level of each target gene was normalized to GAPDH gene as an endogenous reference. The data obtained were analyzed with the ABI PRISM 7900 HT Sequence detection system (Bulletin No. 2, Applied Biosystems). For control reactions the same master mixes were used but excluding the template RNA. Synchronization of Primary Schwann Cell and Kinase Inhibition Schwann cells that had been cultured for 30 days were synchronized for 48 hours by removing growth factor and serum. Schwann cells were then released to progress through the cell cycle by addition of 10% serum to the culture. Cytoplasmic and nuclear extracts obtained from these synchronized cells were prepared before (time 0) and after addition of serum at various time points (i.e., 9 to 18 hours after release). For Erk1/2 and pI3-k inhibition, pre-evaluated concentrations of specific MEK1/2 inhibitor UO126 (15 μM) and PD098059 (15 μM), pI3-k inhibitor LY294002 (15 μM) (Cell Signaling Technology) and SOS inhibitor (10 μg) (Upstate Biotechnology, Lake Placid, N.Y.) were added to the culture medium during synchronization and serum release, and cytoplasmic and nuclear extracts were prepared as described above and the proteins were immunoblotted using antibodies to phospho-Erk1/2, phospho-Akt, phosphoGSK-3β and cyclin-D1. For SOS inhibition studies, cell permeable SOS inhibitory peptide was added and the cells were treated as described above to analyze the effect of SOS inhibitor on M. leprae-induced activation of the above-referenced signaling molecules. None of the inhibitors examined produced any detectable apoptosis or non-apoptotic Schwann cell death under these conditions.

M. leprae-infected and control Schwann cells were also treated with the CDK inhibitors Olomoucine and Roscovitine (Sigma, St. Louis Mo.) that specifically block CDK1, CDK2 and CDK5 functions (Vesely et al., 1994, Eur. J. Biochem. 224: 771-786; Abraham et al., 1995, Biol. Cell. 83: 105-120). Olomoucine and Roscovitine were dissolved in DMSO and were added to the cell cultures at pre-evaluated concentrations of 50 and 100 μM for Olomoucine and 10 and 20 μM for Roscovitine. Cells were harvested 24 hours after treatment with either inhibitor and the cell cycle profiles were analyzed by FACS as described elsewhere herein.

MAP Kinase Activity Assay

MAP kinase activity in infected and non-infected cells was determined by assessing the capacity of a p44/42 (Erk1/2) to phosphorylate Elk-1 in vitro using p44/42 activity assay kit per manufacturer's instructions (Cell Signaling Technology, Beverly, Mass.). Briefly, phosphorylated p44/42 was immunoprecipitated from M. leprae-infected and non-infected human Schwann cell lysates with an immobilized monoclonal phospho-antibody to p44/42 Map kinase (Cell Signaling Technology). The resulting immunoprecipitate containing active MAP kinase was incubated with an Elk-1 fusion protein in the presence of 50 μM ATP and kinase buffer (25 mM Tris pH 7.5, 5 mM β-Glycerolphosphate, 2 mM DTT, 0.1 mM Na₃VO₄, 10 mM MgCl₂). Immunoprecipitated active MAP kinase was then allowed to phosphorylate Elk-1. Phosphorylation of Elk-1 at Ser 383, a major phosphorylation site by MAP kinase, was measured by western blotting using a phospho-Elk-1 (Ser 383) antibody according to manufacturer's instructions (Cell Signaling Technology).

Transcription Factor Array

To identify potential transcription factors associated with the regulation of human Schwann cell cycle during M. leprae infection, the TranSignal Protein/DNA Array (Panomics Inc., Redwood City, Calif.) was used per manufacturer's instructions. That is, nuclear extracts from control and infected Schwann cells were prepared according to the protocol provided by the manufacturer, and the extracts were incubated with a set of biotin-labeled DNA binding oligonucleotides (TranSignal Probe Mix, Panomics Inc.). The resulting DNA/protein complexes, if any, form, were then hybridized to the TranSignal Arrays spotted with different human consensus-binding sequences each corresponding to a specific transcription factor. Binding of proteins present in the nuclear extract to the array was detected by incubating the arrays with Streptavidin-conjugated HRP, and normalization and analysis were performed using a densitometer (Alpha Innotech Corporation, San Leandro, Calif.) and Alpha Imager 2200/5.5 software all as described by the manufacturer.

Transfection of Primary Human Schwann Cells

For transient transfection of a cyclin D1 mutant gene, semi-confluent (approximately 60%) 30 day-infected and control human primary Schwann cells were transferred to 60-mm-diameter dishes, and pFLEX-D1 and the pFLEX-T156A constructs (Diehl and Sherr, 1997, Mol. Cell. Biol. 17: 7362-7374) were introduced using the calcium phosphate method per manufacturer's instructions (Promega, Madison, Wis.). Reporter gene expression was detected using the pCMV.SPORT-βgal vector (Invitrogen Life Technologies). Transfections were terminated after 48 hours and the cells were assayed to determine transfection efficiencies (β-galactosidase staining). Briefly, transfected cells were counted using a light microscope over 10 contiguous fields and the average percentage ±STDEV of the β-galactosidase positive cells was assessed. The effect of transfection of cyclin D1 mutant on Schwann cell cycle progression was determined using cell cycle FACS as described previously elsewhere herein.

The Results of the experiments disclosed herein are as follows.

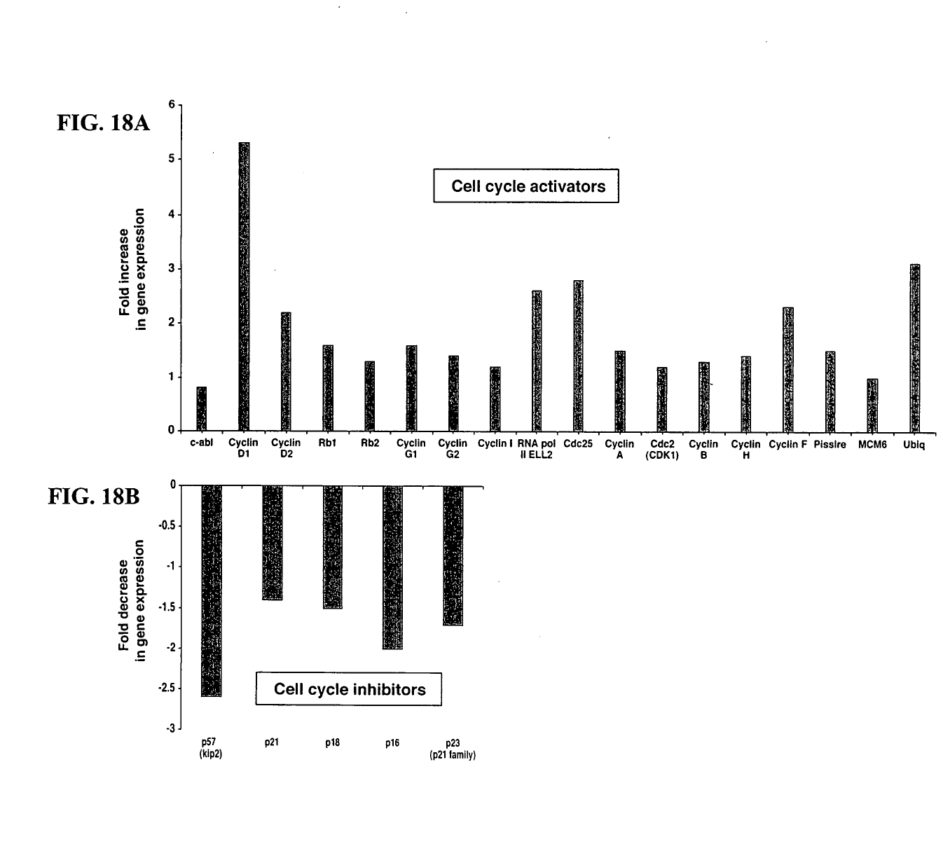

Genearray data indicates that intracellular M. leprae mainly induces upregulation of cell cycle activators and co-activators that mediate the G1 to S-phase transition, particularly cyclin D1, which is a major known regulator of cellular proliferation (FIG. 18). At the same time, M. leprae infection mediates the down-regulation of genes encoding critical eukaryotic cell cycle inhibitors, such as p57, p23, p21, p18, and p16 (FIG. 18). These data indicate that progression through the cell cycle is sustained during M. leprae infection without any inhibitory effects.

Additionally, intracellular M. leprae upregulates genes for two of the major signaling kinases, Erk1/2 and PI3/p85. At the protein level, Erk1/2 is significantly phosphorylated by M. leprae. Activation of these kinases is known to be indicative of cellular proliferation response. Further, cyclin D1 is a downstream target of the Erk1/2 signaling pathway, suggesting a putative novel pathway, but MEK/Pi3K-independent phosphorylation of Erk1/2 by intracellular M. leprae for suggests a putative novel pathway for M. leprae induced cell proliferation (FIGS. 19 and 20).

Previous studies demonstrated that cell cycle progression is controlled, in part, at the level of gene transcription. Most of the transcriptional control involves phosphorylation by specific cyclin/CDK pairs, which act as transcriptional activators or repressors. In order to detect the specific transcription factors in the nuclear extracts of M. leprae infected Schwann cells, a transcription factor array and electromobility gel shift assays (EMSA) were employed. The binding was specifically competed with unlabelled (cold) Ets or CREB probes. Both transcription factors are negative in uninfected Schwann cell nuclear extracts. The transcription factors E2F, CREB, and Ets are significantly upregulated in M. leprae-infected Schwann cells. CREB and Ets are known to regulate cell survival and proliferation, and it is likely, without wishing to be bound by any particular theory, that these transcription factors play a crucial role in M. leprae-induced cell cycle progression. Further, E2F, CREB, and Ets are down stream targets of the Erk 1/2 signaling pathway, and therefore, M. leprae apparently plays a role in the complex signaling, transcriptional control, and activation of cyclin/CDKs.

As demonstrated by the data disclosed herein, M. leprae employs the induction of growth, neurotrophic, or both, factors in Schwann cells, leading to the ES-like phenotype, increased proliferation, and capacity to re-differentiate into various cell types. Most of the growth/neurotrophic factors are from the IGF, FGF, and NGF families (FIG. 3B). These growth factors are known to play a critical role in cell growth, proliferation, differentiation and nerve regeneration.

Among the many growth/neurotrophic factors upregulated, FGF-7 and IGF-1 show approximately 48 fold increases in M. leprae infected Schwann cells (FIG. 3B). IGF-1 is a strong promoter of glial cell development, and strongly stimulates Schwann cell survival and proliferation. Further, IGF-1 also serves as a major neurotrophic factor and plays a neuroprotective role in several neurodegenerative diseases. FGF-7 has never before been detected in Schwann cells or glial cells in the CNS. In order to confirm the genearray data, real-time quantitative PCR was employed to analyze the upregulation of IGF-1 and FGF-7 in 30 day post-infection human Schwann cells. The expression of IGF-1 and FGF-7 was normalized to the house keeping gene GAPDH for both infected Schwann cells and uninfected controls. Real-time PCR confirmation of the genearray data confirmed the massive upregulation of IGF-1 and FGF-7 in infected Schwann cells as compared to uninfected cells.

In order to further elucidate the pathway M. leprae uses to facilitate Schwann cell proliferation without transformation, Schwann cells were synchronized in the cell cycle, and then released. Specific kinase inhibitors (MEK1/2 inhibitor U0126 and PD098059, PI3-K inhibitor LY294002, SOS inhibitor, CDK1, CDK2, CDK5 inhibitors olomoucine and roscovitine) were administered to Schwann cell cultures during synchronization and serum release. Cytoplasmic and nuclear extracts were prepared as described above and immunoblotted with antibodies to the phosphorylated forms of the key cell cycle regulators Erk1/2, Akt, GSK-3β, and cyclin D1.

The results of these experiments demonstrated that a novel intracellular signal, as opposed to the dogmatic concept of the necessity of extracellular receptor-mediated cell cycle initiator, determined the cell cycle entry and progression through the G1 phase in M. leprae infected Schwann cells. The classical receptor-mediated extracellular mitogen/growth factor-induced PI3 kinase and Ras>Ref>MEK1/2 signaling pathways that regulate nuclear accumulation of cyclin D1 and subsequent cell cycle progression and proliferation are depicted, as are the points where M. leprae bypasses or subverts this mechanism to induce cellular proliferation without transformation. In the classical pathway, an extracellular growth factor and/or mitogen induces a series of phosphorylation and activation events leading to the transcription and accumulation of cyclin D1 in the cell nucleus. FIGS. 19 and 20 depict that despite the inhibition of the activity of the upstream targets PI3K with LY294002, SOS with SOS inhibitor, and MEK1/2 with U0126, M. leprae, or a cellular factor activated by intracellular M. leprae or acting in concert with an M. leprae component, activates GSK-3β and ERK1/2 by phosphorylation, leading to the continuous and controlled progression of cells through the cell cycle, leading to proliferation without transformation. Even in the complete absence of serum and growth factors, M. leprae phosphorylates Erk1/2 and GSK-3β.

Surprisingly, specific SOS, MEK, and PI3K inhibitors that are known to block downstream signaling of Ras and PI3K-dependent signaling do not inhibit the novel M. leprae-induced phosphorylation. The data disclosed herein demonstrate that M. leprae controls the cell cycle and affect cell proliferation through a novel signaling pathway different from the canonical pathways (FIG. 20).

Moreover, sustained activation of ERK1/2 by intracellular M. leprae results in the transcription of cyclin D1, as evidenced by the increased presence of cyclin D1 transcripts in the genearray data disclosed elsewhere herein. Further, the transcription of cyclin D1 leads to the formation of the cyclin D1/CDK4 holoenzyme complex that drives cell cycle progression leading to the continuous proliferation without transformation as demonstrated by the data disclosed elsewhere herein. Thus, the data disclosed herein demonstrate that Erk1/2 and GSK3β phosphorylation by a novel signaling pathway described herein mediates change in the cell fate, resulting in unbridled, yet non-malignant transformed, cell proliferation characteristic of stem cells. Therefore, this novel MEK/Pi3K-independent pathway could play a critical role in reprogramming process.

### Example 5

### Re- and Trans-Differentiation of an ES-Like Cell

The data disclosed herein demonstrate that human cells can be reprogrammed into a precursor, ES-like cell state that, where the cells, among other things, proliferate continuously, express genes characteristic of stem cells, and exhibit down regulation of genes characteristic of a highly differentiated and specialized adult cell. Further, the ES-like cell can differentiate into another cell type of different lineage than original glial cells (ectodermal), referred to herein as “trans-differentiation”. That is, the ES-like cells of the present invention display some of the characteristic traits of embryonic stem cells in that they proliferate continuously in culture without exhibiting a transformed malignant phenotype and they can trans-differentiated into other cell types. The teachings disclosed in this Example demonstrate methods for the generation of an ES-like stem cell and methods for re-differentiating the cell, i.e., first reprogram the cell and then convert to an ES-like cell and then re-differentiate or trans-differentiate the cell, into adult cell types of the same or different lineage. Further, the present disclosure presents data disclosing the striking similarities between both ES-like cells and ES cells and differentiated ES-like cells and their existing counterparts.

To determine whether these reprogrammed cells possess the properties of neural stem cell-like progenitor cells, the following were assessed: (1) the capacity of reprogrammed cells to form neurospheres, (aggregates of highly proliferative cells), which is a known characteristic feature of stem cells in culture, (2) the self-renewal capacity of the reprogrammed cells, and (3) the differentiation capacity of neurospheres to form neuron- and oligodendrocyte-like cells.

Production of Primary Neurospheres from Reprogrammed Adult Human Primary Schwann Cells

Adult human primary Schwann cells were isolated and purified from different human donors and were infected with M. leprae for 30 days. Infected cells after 30 days (“reprogrammed” cells on the basis of genetic makeup of these cells) were incubated with bFGF and chicken embryo extract in neural stem cell media without serum. Neural stem cell medium/progenitor medium comprising of DMEM/F12, 1% N2 supplement, 2% B27 supplement, 10% chick embryo extract and 20 ng/ml bFGF as described in Morrison et al. (1999, Cell 96:737-749) was added. After 10 days the cells started lifted off and form small aggregates. Over time, cells in these aggregate underwent continuous proliferation and formed highly dense neurospheres, phenotypically similar to ES-cells in culture.

For production of primary neurospheres, ES-like cells were treated with a progenitor medium. The medium was replaced every 4 days for 21 days. At day 21, floating neurospheres were removed using a sterile Pasteur pipette and plated on poly-lysine/laminin coated coverslips to assess differentiation (1 neurosphere per coverslip) or the cells were propagated to produce secondary neurospheres. Neurospheres were probed for nestin expression using the immunofluorescence protocol described elsewhere herein.

For secondary neurosphere formation, primary neurospheres were dissociated using a fire-polished Pasteur pipette and the cells were plated as individual cells in uncoated 96-well plates using progenitor medium. The medium was refreshed every 4 days and the cells were monitored for cell death and for self-renewal. Secondary neurospheres were kept in culture for a total of 20 days until they were ready to be differentiated according to the methods described in Gritti et al. (1996, J. Neurosci. 16: 1091-1100).

Re-Differentiation of Primary and Secondary Neurospheres to Neuron-Like Cells

Neuronal differentiation was performed as follows. Briefly, neurospheres were cultured overnight in DMEM/F12, 2% defined fetal bovine serum (FBS) to assist neurosphere attachment to the substrate, and the medium was changed to DMEM/F 12 with 1% N2, 2% B27, 2 μM Forskolin, 20 ng/ml BDNF and 20 ng/ml NT3 (neural differentiation media) and the cells were maintained in this media for a total of 15 days. Neuronal differentiation was monitored using immunofluorescence using a panel of antibodies specific for various stages of neuronal development (i.e., NeuN, Neurofilament-L, MAP2 and βIII-Tubulin (TuJi)); as described in Morrison et al. (Cell 96; 737-749); Gritti et al. (1996, J. Neurosci. 16: 1091-1100); Kalyani, et al. (1997 Dev. Biol. 186: 202-223); Studer et al. (1998, Nature NeuroScience 1: 290-295). Antibodies specific for Glutamate Receptor-1, NeuN and phospho-Neurofilament-H were used as mature neuronal markers.

Differentiation of Neurospheres to Oligodendrocyte-Like Cells

To differentiate ES-like cells into oligodendrocytes, neurospheres were plated in poly-L-lysine/laminin coated coverslips with DMEM, 1% N2 supplement, 2% defined FBS and 10 ng/ml PDGF-AA (oligodendrocyte differentiation media) for a total of 20 days. Oligodendrocyte differentiation was monitored by immunofluorescence using an anti-O4 specific polyclonal antibody. As a positive control for anti-O4, rat spinal cord explant cultures in which all spinal cord oligodendrocytes were labeled with anti-O4 were used.

The Results of the experiments disclosed herein are as follows.

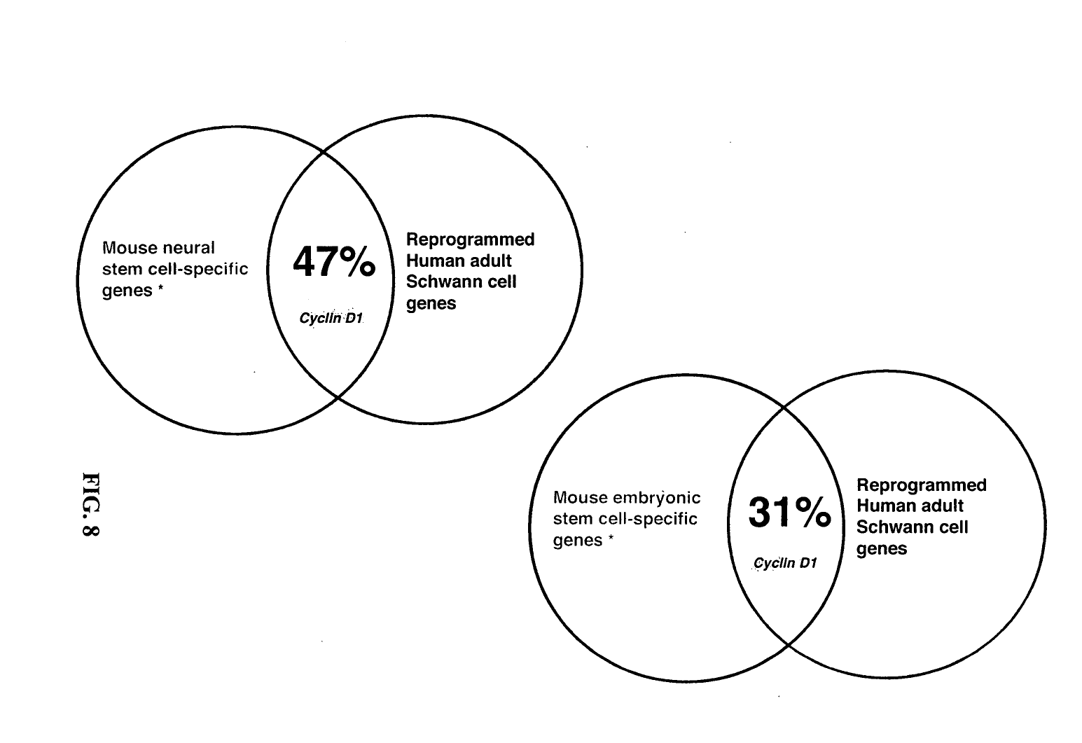

The data disclosed herein demonstrates that reprogrammed human Schwann cells express genes consistent with an ES-like phenotype and a loss of differentiation. As summarized in Tables 3 and 4 and FIG. 8, the data disclosed herein demonstrate for the first time, that reprogrammed human adult Schwann cells (30 days after infection with M. leprae) produced according to the methods of the present invention expressed many of the known mouse neural stem cell-specific genes and many of the mouse embryonic stem cell-specific genes (Ivanova et al., 2002, Science 298: 601-604; Ramalho-Santos et al., 2002, Science 298: 597-600). That is reprogrammed human Schwann cells express about 47% of the mouse neural stem cell-specific genes, including cyclin D1. Reprogrammed human Schwann cell expressed about 31% of the mouse embryonic stem cell-specific genes, including cyclin D1 (FIG. 8). These data demonstrate that even at the reprogrammed stage, before forming neurospheres, these cells possess the genetic makeup and potential to become ES-like cells. That is, even before forming neurospheres (see, e.g., step I and II as depicted in FIG. 17), the reprogrammed cells already express numerous ES-cell and stem cell genes. These results demonstrate that reprogrammed cells (ES-like cell after conversion of reprogrammed cell to ES-like cells that are floating and form spheres) have the genetic make-up to become actual stem cell-like cells.

TABLE 1
Reprogrammed human Adult Schwann Cells Express Genes That Are
Highly Enriched in Undifferentiated Human Embryonic Stem Cell (HESC)

| | Most significant undifferentiated HESCs- | | Re-programmed |
|---|---|---|---|
| Unigene | enriched genes | Symbol | cells genes |
| 278239 | Left-right determination factor | LEFTB | |
| 2860 | POU domain, class 5, transcription factor 1 | POU5F1 | +* |
| 282387 | FLJ21837 | | |
| 379090 | FLJ38338 | | |
| 2288 | Visinin-like 1 | VSNL1 | |
| 274681 | Adenylate kinase 3 | AK3 | +* |
| 82110 | PC4 and SFRS1 interacting protein 2 | PSIP2 | |
| 5243 | Hypothetical protein, estradiol-induced | E2IG5 | |
| 124027 | Selenium donor protein | SPS | |
| 180403 | STRIN protein | STRIN | |
| 1907 | Galanin | GAL | +* |
| 180383 | Dual specificity phosphatase 6 | DUSP6 | +* |
| 48269 | Vaccinia related kinase 1 | VRK1 | |
| 9536 | FLJ10713 | FLJ10713 | |
| 61638 | Myosin X | MYO10 | +* |
| 112360 | Prominin-like | PROML1 | |
| 140720 | Frequently rearranged in advanced T-cell | FRAT2 | |
| | lymphomas 2 | | |
| 1787 | Proteolipid protein | PLP1 | |
| 90093 | Heat shock 70 kDa protein 4** | HSPA4 | +* |
| 1608 | Replication protein A3** | PRA3 | +* |
| 182877 | KIAA0116 protein** | KIAA0116 | +* |
| 194562 | Telomeric repeat binding factor** | TERF-1 | +* |

*Highly significant; p = <0.005
**Also represent as signature stem cell genes in mouse embryonic stem cells

TABLE 2
Common Signaling-related Genes Between Undifferentiated HESCs
(Enriched Genes) and Reprogrammed Human Adult Schwann Cells

| | | Signaling- |
|---|---|---|
| | | related |
| | | genes in |
| Signaling-related genes enriched in | | reprogrammed |
| undifferentiated HESCs | Symbol | Schwann cells* |
| Basic fibroblast growth factor | bFGF | +* |
| Fibroblast growth factor receptor 1 | FGFR1 | +* |
| Fibroblast growth factor receptor 2 | FGFR2 | |
| Fibroblast growth factor receptor 4 | FGFR4 | |
| Inhibitor of FGF signaling | Sprouty-1 | +* |
| Inhibitor of FGF signaling | Sprouty-4 | |
| Transforming growth factor-beta | TGFβ | +* |
| Transforming growth factor-beta receptor | TDGF1 | +* |
| Secreted inhibitors of TGF signaling | Lefty A & B | |
| TGFβ-induced factor | | |
| Bone morphogenetic protein | BMP | +* |
| Wnt protooncogene | Wnt | +* |
| Wnt receptor | Frizzled | +* |

*Highly significant; p = <0.005

Recent studies have identified the transcriptional profiles of undifferentiated human embryonic stem cell (HESC) and showed that HESC shared a common stem cell genetic program with mouse embryonic stem cell and mouse adult neural stem cells (Sato et al., 2003, Dev. Biol. 260:404-413). Genetic profiling of HESC that give rise to the “stemness” of these cells demonstrate expression of genes of 15 ligand/receptor pairs and secreted inhibitors of the FGF, TGFB/BMP and Wnt pathways. Interestingly, reprogrammed adult human Schwann cells that are treated with M. leprae for 30 days express a significant number of above-mentioned critical sets of genes that define the “stemness” of human ES cells. Comparison of signaling related genes of HESC reveal an impressive 61% of identity with the genes expressed by reprogrammed cells. These data suggest a common stem cell genetic program between reprogrammed adult cells and human ES cells and demonstrate the potential of reprogrammed adult cells of the invention to function like human ES cells such as, but not limited, in their potential to differentiate into cells of all three germ layers.

Nonetheless, at the ES-like stage, the reprogrammed cells do not behave like canonical stem cells and do not possess morphological features characteristic of stem cells in vitro, thus, are only considered to have the “potential” to become stem cell-like cells requiring a conversion step (step II as illustrated in FIG. 17). The conversion was achieved by exposure of the reprogrammed cells to progenitor cell media. Only stem cell-like cells survive and proliferate in this medium, since it does not contain serum and other supplements required for normal somatic or non-stem cell growth. Therefore, growth of the reprogrammed cells in progenitor cell medium demonstrates that the cells have achieved stem cell-like cell stage, acquiring morphological, phenotypical, highly proliferative (more proliferative than attached reprogrammed cells), genetic and behavioral features similar to embryonic stem cells.

TABLE 3
Mouse Neural Stem Cell-Specific Genes Expressed in Reprogrammed
Human adult Schwann Cells

| | | Present in |
|---|---|---|
| | | Reprogrammed |
| Gene Name | Gene Description | Schwann Cells |
| Igfbp3 | Insulin like growth factor binding | Yes |
| | protein 3 | |
| Fyn | Fyn proto-oncogene | |
| Ccnd2 | Cyclin D2 | Yes |
| Hsd17b12 | Hydroxysteroid dehydrogenase 12 | Yes |
| Ccnd1 | Cyclin D1 | Yes |
| Scd2 | Stearyoyl-coA desaturase 2 | |
| Slc1a4 | Solute Carrier Family 1, member 4 | |
| Cfl2 | Cofilin 2, muscle | |
| Eno1 | Enolase 1, alpha non-neuron | |
| Adh5-ps1 | Alcohol dehydrogenase 5 | Yes |
| Tdag | T-cell death associated gene | |
| Cox7a3 | Cytochrome C oxidase, subunit VIIa 3 | Yes |
| Stc2 | Stanniocalcin 2 | Yes |
| Rab2 | RAB2, member Ras oncogene family | Yes |
| Hnrpd1 | Heterogenous nuclear riboprotein | Yes |
| Limd1 | LIM domain containing 1 | Yes |
| Cct2 | Chaperonin subunit 2(beta) | |
| S100a10 | S100 calcium binding protein A10 | Yes |
| Arbp | Acidic ribosomal phosphoprotein PO | |
| H2afz | H2A histone family, member Z | Yes |
| Tuba6 | Tubulin, alpha 6 | |
| Melk | Maternal embryonic leucine zipper kinase | |
| Cct5 | Chaperonin subunit 5 (epsilon) | |
| Smt3h1 | SMT3 homolog 1 | |
| Racgap1 | Rac GTPase-activating protein | |
| Ap3s1 | Adaptor related protein complex AP-3 | Yes |
| Ifi203 | Interferon activated gene 203 | |
| Ercc1 | Excision repair, complementation group 1 | Yes |
| Arhj | Ras homolog gene family, member J | |
| Nrtn | Neuritin | |

TABLE 4
Mouse ES Cell-Specific Genes Expressed in Reprogrammed Human
adult Schwann Cells

| | | Present in |
|---|---|---|
| | | Reprogrammed |
| Gene Name | Gene Description | Schwann Cells |
| Pou5f1 | POU domain, class 5, transcription factor | Yes |
| | (5 different POU domains are expressed | |
| | in reprogrammed Schwann Cells | |
| Nmyc1 | Neuroblastoma myc-related oncogene-1 | |
| Gbx2 | Gastrulation brain homeobox 2 | |
| FGF4 | Fibroblast growth factor 4 | |
| Ung | Uracil-DNA glycosylase | Yes |
| Tcea3 | Transcription elongation factor A (SII), 3 | Yes |
| Pea3 | Polyomavirus enhancer activator 3 | |
| Mybl2 | Myeloblastosis oncogene-like 2 | |
| Klf4 | Kruppel-like factor 4 (gut) | |
| Ccnd1 | Cyclin D1 | Yes |
| Lamr1 | Laminin receptor 1 | Yes |
| Odc | Ornithine decarboxylase | Yes |
| Pecam | Platelet/endothelial adhesion molecule | |
| Ptma | Prothymosin alpha | |
| Klf2 | Kruppel-like factor 2 (lung) | |
| Ccne1 | Cyclin E1 | Yes |
| Ttf1 | Transcription termination factor 1 | |
| Hsp86-1 | Heat shock protein, 86 kDa 1 | |
| SCF | Stem Cell Factor (Hematopoietic stem | Yes |
| | cells) | |

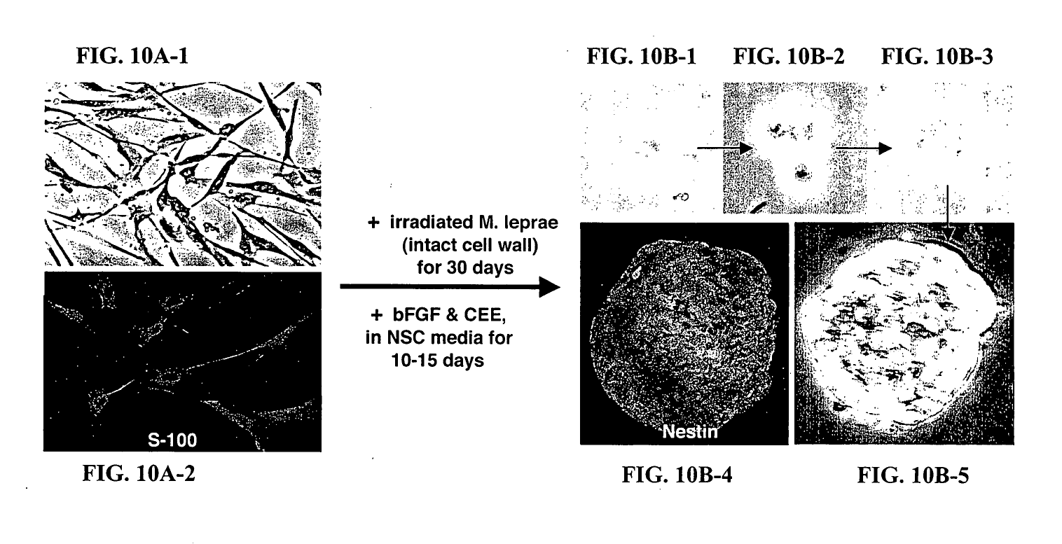

Due to the multiple morphogenic and genetic similarities between reprogrammed adult Schwann cells and ES cells, especially neural ES cells, the reprogrammed cells were cultured in the same manner as described previously for neural ES cells, this incubation is also referred to herein as “conversion” (see FIG. 17, step II). Thus, in the studies detailed herein, the well-established protocols for propagation of neural stem cells were used (Morrison et al., 1999, Cell 96: 737-749; Gritti et al., 1996, J. Neurosci. 16: 1091-1100; Kalyani et al., 1997, Dev. Biol. 186: 202-223; Studer et al., 1998, Nature NeuroScience 1, 290-295). After 10 days in culture the first small floating neurospheres appeared and grew substantially larger in the presence of bFGF-containing medium (FIG. 9). Further, infecting Schwann cells with irradiated M. leprae with intact cell walls resulted in the formation of primary neurospheres under the same conditions (FIG. 10). All neurospheres were positive for neural stem cell marker nestin, as demonstrated by specific antibody to nestin (FIG. 10B-4). The presence of a marker known previously to be specific for neural stem cell (Morrison, et al., supra; Gritti et al., supra; Studer, et al., supra), nestin, further confirmed that these cell aggregates are indeed neurospheres.

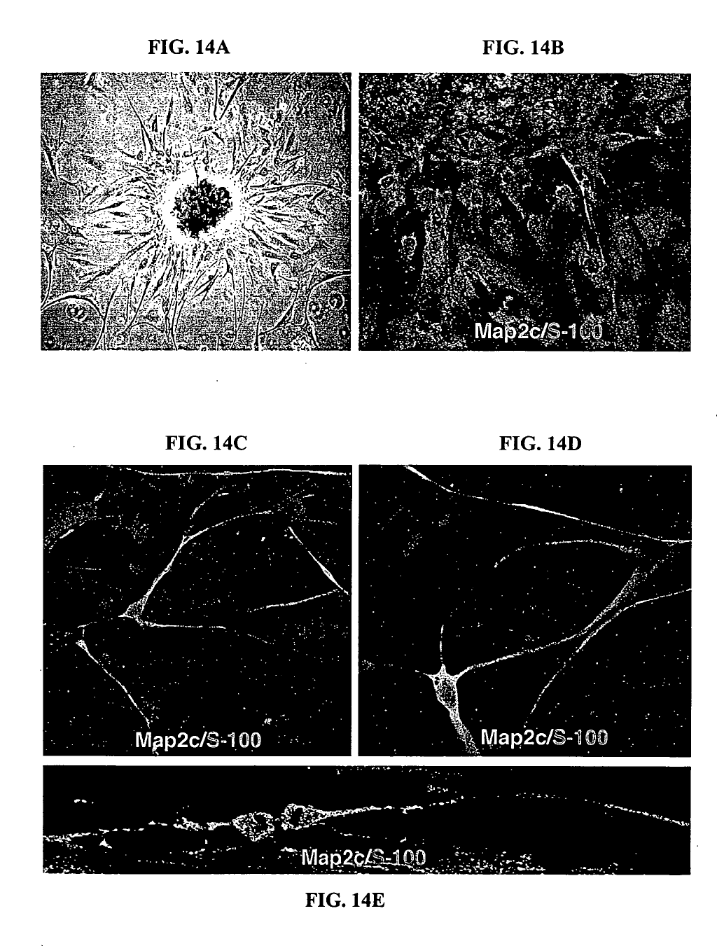

Secondary neurospheres were capable of self-renewal. That is, proliferative cells were clearly evident after 12 days in culture when small floating secondary neurospheres were starting to form. These secondary neurospheres were also positive for nestin, and were cultured until differentiation media was added. ES-like cells generated from reprogrammed human Schwann cells exhibited all the characteristics of neural stem cells, including their capacity to form neurospheres, self-renewal, differentiation into neuron-like cells and the presence of nestin (FIGS. 9 and 14, respectively).

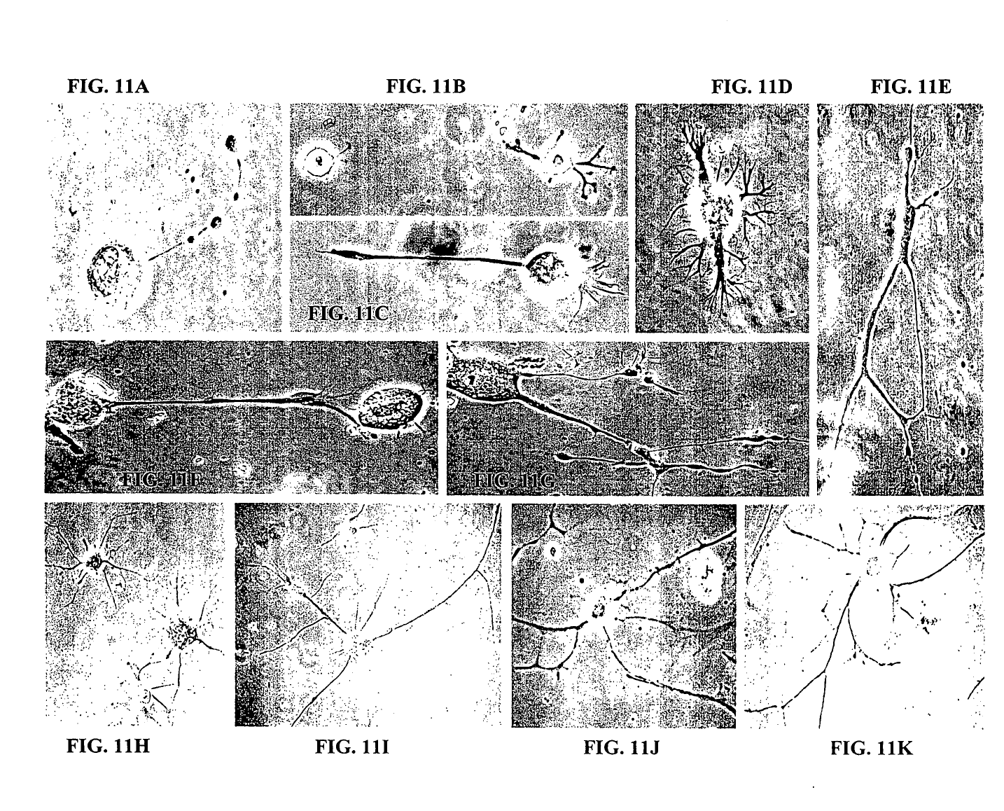

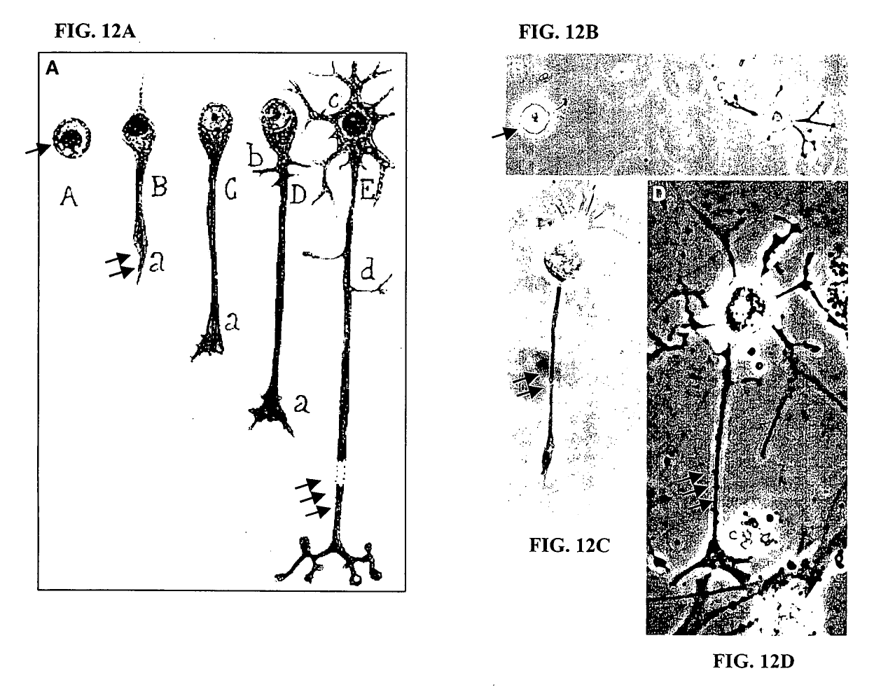

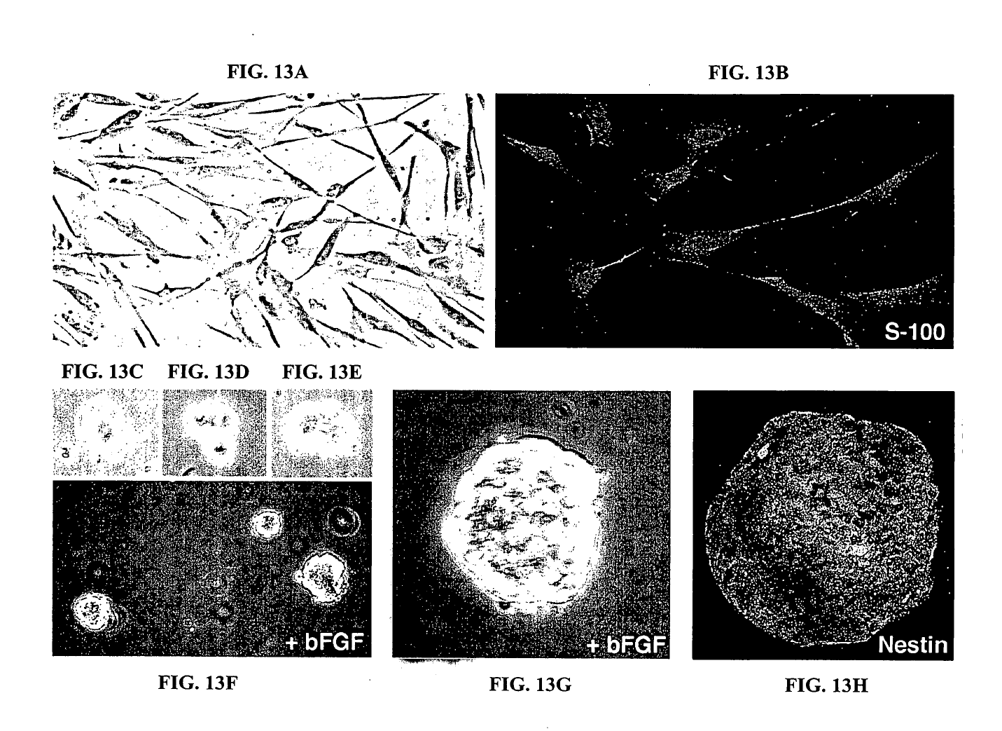

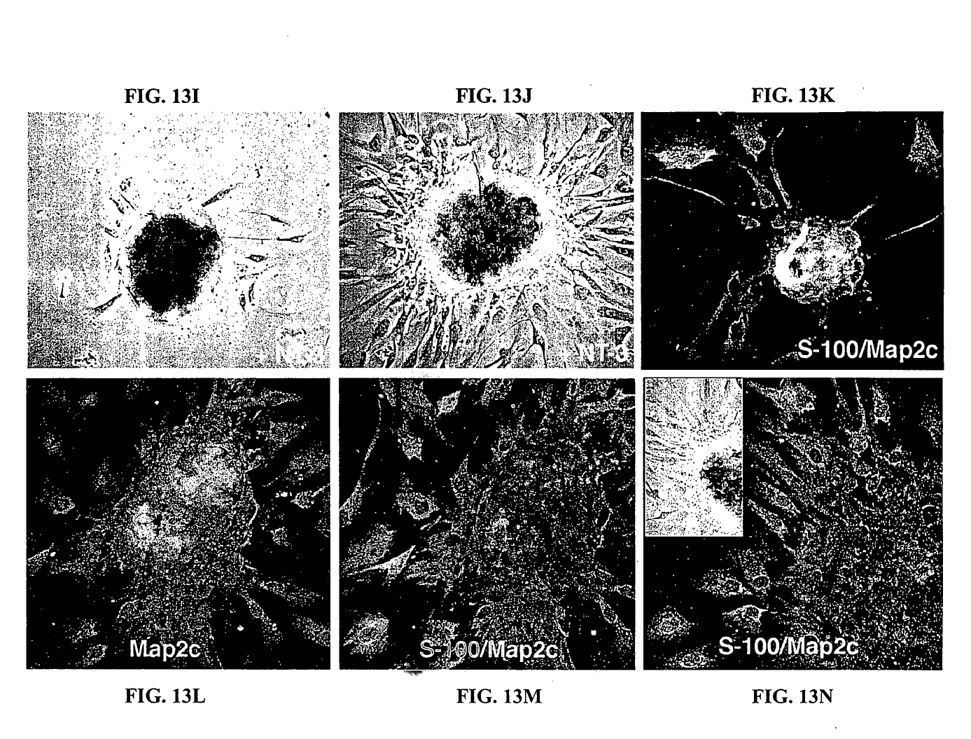

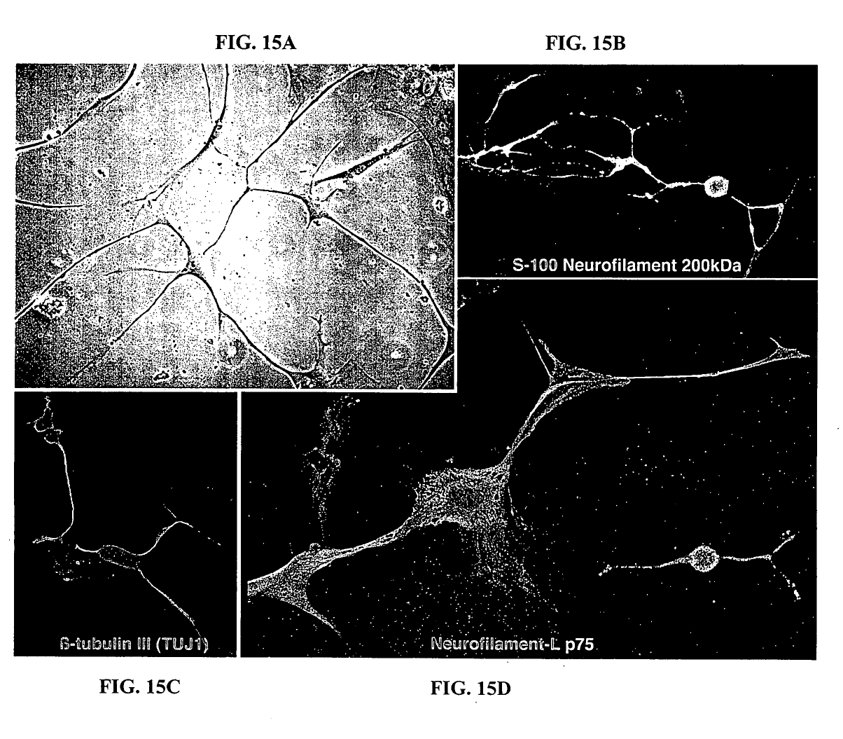

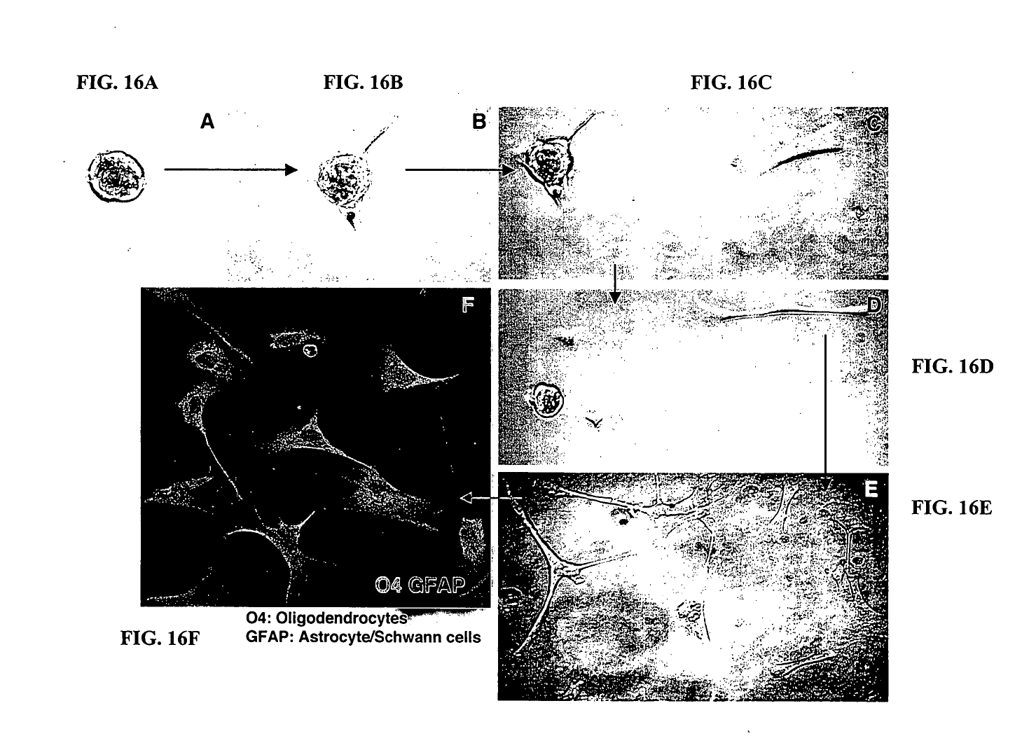

In vitro differentiation of secondary neurospheres resulted in the formation of neuron-like cells. Reprogrammed Schwann cells differentiate into neuron-like cells with long axon-like processes, and their development was remarkably similar to the development of mature neurons from undifferentiated neuronal progenitors (FIGS. 11 and 12, respectively). Further, in order to monitor the development of neuronal cells, a panel of antibodies comprising of antibodies to NeuN, Neurofilament-L, MAP2, and βIII-Tubulin was used. The markers employed in the present study are known to be characteristic of neural progenitor cells developing into mature neurons (Morrison, et al., supra; Gritti et al., supra; Kalyani, et al., supra; Studer, et al., supra), and the immunostaining by these antibodies further demonstrated that the cells of the invention were neural progenitor cells (FIGS. 13-15). Further, developing neuron-like cells eliminate expression of the Schwann cell marker S-100 and express the neuron marker MAP2 (FIG. 14). These data clearly indicate that ES-like cells generated by reprogramming adult differentiated cells posses the properties of neural stem cell-like cells, and have the capacity to differentiate to mature neuron-like cells.

Secondary neurospheres were also differentiated into oligodendrocytes, the myelin forming glial cells of the central nervous system, when exposed to media containing PDGF using the methods described above. Oligodendrocyte differentiation was monitored using antibodies to O4, an oligodendrocyte-specific marker. These findings indicate that the reprogrammed cells not only have the capacity to differentiate into neurons but also to oligodendrocytes, the myelin-forming glial cells of the central nervous system. The ability to produce human oligodendrocytes provides an important potential therapeutic for the regeneration of myelin sheaths in demyelinating disease, such as, but not limited to, multiple sclerosis.

### Example 6

### Novel Signaling Mechanism of G1/S Phase Transition of Human Glial Cell-Cycle

The G1/S phase transition of the mammalian cell cycle normally depends on stimulation by receptor-mediated signaling from extracellular cues. It has been demonstrated that stimulation by signaling from an intracellular cue alone can promote G1/S phase transition. By using Mycobacterium leprae as an intracellular cue, the data disclosed herein demonstrate, for the first time, that M. leprae residing inside human primary Schwann cells modulated the transcription of key G1 phase regulators and induced phosphorylation of Ser-158 residue of p56Lck (Lck/Ser-158) that directly phosphorylated Erk1/2 (p42/44 MAPK) by a novel MEK/Pi3-kinase-independent and PKC-alpha/betaII/βII-dependent signaling pathway.

Activation of Erk1/2 via this novel pathogen-induced pathway resulted in nuclear accumulation of cyclin-D1, G1/S phase progression and continuous Schwann cell proliferation without transformation. Thus, Lck/Ser-158 serves as a previously unknown activator of Erk1/2 in glial cells, and mediates Erk1/2-induced G1/S phase progression independent of MEK1/2 and Pi3-kinase pathways. Thus, the data disclosed herein reveal a novel signaling mechanism of G1/S phase transition in mammalian cells and demonstrate how an obligate intracellular bacterial pathogen propagates its preferred niche for long-term survival. These data provide novel methods for proliferating cells and for producing large numbers of undifferentiated and/or dedifferentiated cells for use in, among other things, cell-based therapies. Additionally, these data provide important methods for treating leprosy mediated by pathogen control of these novel signaling pathways.

Proliferation of glial cells, both Schwann cells in the peripheral nervous system and oligodendrocytes in the central nervous system, is crucial for the development of nervous system and nerve regeneration as well as tumorgenesis in the peripheral nerves and in the brain (Jessen and Mirsky, 1999, Ann. New York Acad. Sci. 883:109-115; Keistead and Blakemore, 1999, Adv. Exp. Med. Biol. 468:183-197; Kim et al., 2000, Neuron 26:405-416; Miellinen et al., 2001). Despite the importance of these cells, little is known about regulation of the glial cell cycle, and in particular, no data are available about the molecular biology of human Schwann cell cycle in general.

During the progression of the mammalian cell cycle, cells transit through G1, S, G2 and M phases, and complete the cycle resulting in daughter cells with identical chromosomal copies (Pardee, 1989, Science 246:603-608; Sherr, 1993, Cell 73:1059-1065; Hunter and Pines, 1994, Cell 79:573-582). G1 phase progression, unlike S, G2 and M phases of the cell cycle, normally depends on stimulation by extracellular cues such as mitogens or growth factors (Pardee, 1989, Science 246:603-608; Sherr, 1993, Cell 73:1059-1065; Sherr, 2000, Cancer Res. 60:3689-3695). Neurotrophic pathogens that infect glial cells and reside intracellularly may interfere with or subvert glial cell cycle machinery for their own survival advantage, and thus serve as attractive models to study the regulation of glial cell cycle.

One such pathogen is Mycobacterium leprae, a causative organism of leprosy, and the only known human bacterial pathogen that preferentially invades Schwann cells (Stoner, 1979, Lancet 2:994-996; Mukherjee, and Antia, 1986, Int. J. Lepr. 54:632-638). Remarkably, with a limited set of functional genes in its genome, leprosy bacillus is highly adapted to long-term intracellular survival by subverting Schwann cell functions without causing apparent host cell death (Cole et al., 2001, Nature 409:1007-1011; Rambukkana et al., 2001; Rambukkana et al., 2002, Science 296:927-931; Brophy, 2002, Science 296:862-863). Because M. leprae is a non-toxic/non-cytolytic bacterium and possesses unique properties (Rambukkana et al., 1997, Cell 88:811-821; Rambukkana, 2004, Curr. Opin. Immunol. 16:(in press)), using M. leprae and its products as tools to dissect the biology of neural cell functions has been of interest.

Recently, it was reported that myelinating and non-myelinating Schwann cell phenotypes show distinct functional responses to M. leprae infection in a rat nerve tissue culture model. That is, M. leprae attachment to myelinated and non-myelinated Schwann cell-axon units specifically induces demyelination and bacterial invasion, respectively (Rambukkana et al., 2002, Science 296:927-931). Non-myelinated Schwann cells are mostly susceptible to M. leprae invasion and preferentially harbor M. leprae in vitro (Rambukkana et al., 2002, Science 296:927-931) and in the majority of leprosy patients with high bacterial load (Shetty et al., 1980, Lepr. India 52:5-18; Shetty et al., 1988, J. Neurol. Sci. 88:115-131). Subsequent to invasion, M. leprae reside within human Schwann cells for a long period before causing peripheral nerve damage, the hallmark of leprosy that affects millions of people worldwide (Stoner, 1979, Lancet 2:994-996; Mukherjee, and Antia, 1986, Int. J. Lepr. 54:632-638; Job, 1989; WHO, 2002, Weekly Epidemiological Record No. 77). Understanding the molecular mechanisms by which M. leprae subvert Schwann cell functions for long-term intracellular survival not only will facilitate development of effective therapeutics to prevent nerve damage in leprosy, but will also provide novel insights into the basic biology of glial cells and, in turn, provide novel methods for controlling glial cell development as disclosed for the first time herein.

One key to the pathogenic potential of long-term survival of M. leprae within human Schwann cells lies in the ability of this bacterium to propagate its intracellular niche so that sufficient Schwann cells are available for bacterial replication and survival (Rambukkana et al., 2002, Science 296:927-931; Rambukkana, 2004, Curr. Opin. Immunol. 16:(in press); Brophy, 2002, Science 296:862-863). Using highly purified primary human Schwann cells, which mimic non-myelinating phenotypes, the data disclosed herein provide evidence that one effective way of propagating cellular niche lies within the capacity of intracellular M. leprae to induce Schwann cell proliferation from inside the cells. Since mammalian cell proliferation is defined in the art as the increase in cell number resulting from completion of the cell division cycle (Pardee, 1989, Science 246:603-608), the ability of intracellular M. leprae to subvert human Schwann cell-cycle machinery was investigated.

When quiescent G0 cells enter cell cycle, genes encoding D-type cyclins of the G1 phase, D1, D2, and D3, are induced in response to extracellular mitogenic signals in a cell-specific manner (Hunter and Pines, 1994, Cell 79:573-582; Sherr, 1995, Trends Biochem. Sci. 20:187-190). In mouse Schwann cells, cyclin D1 and D2, but not D3, is linked to mitogenic stimulus, whereas proliferative growth of mature Schwann cells has been shown to depend strictly on cyclin D1 (Kim et al., 2000, Neuron 26:405-416; Mathon et al., 2001, Science 291:872-875). Since the D-type cyclins play a critical role as sensors for variety of extracellular signals during the cell cycle entry (Sherr, 1993, Cell 73:1059-1065; Sherr, 2000, Cancer Res. 60:3689-3695), there has been much interest in identifying which signaling pathways control the activities of G1 cyclins and their catalytic partners—cyclin-dependent kinases (CDKs) (Marshall, 1999, Curr. Opin. Cell. Biol. 11:732-736; Roovers and Assoian, 2000, Bioessays 22:818-826; Welsh et al., 2001, Nature Cell Biol. 3:950-957; Chang et al., 2003, Int. J. Oncol. 22:469-480).

Activation of extracellular signal-regulated kinases 1 and 2 (Erk1/2; also known as p44/42 MAP kinase) has been implicated as one of the key elements in inducing D-type cyclins in response to extracellular mitogenic responses, such as growth factors (Roovers and Assoian, 2000, Bioessays 22:818-826; Chang et al., 2003, Int. J. Oncol. 22:469-480). The only known well-characterized activator of Erk1/2 is the dual specific protein kinases MEK1/2 (MAP/ERK kinase 1 and 2), which are activated by Raf family kinases in response to extracellular mitogens (Ballif and Blenis, 2001, Cell Growth Differ. 12:397-408).

In addition, Erk1/2 appears to have a fundamental role in integrating multiple intracellular signals activated by protein kinases such as PKC (Stariha and Kim, 2001, Microsc. Res. Tech. 52:680-688). Erk1/2 can also be directly activated by the non-receptor Src-family protein kinase lymphoid cell kinase (Ick) p56Lck, which is thought to express preferentially in tissues of lymphoid origin and plays a role in T-cell development, proliferation and differentiation (Weiss and Littman, 1994, Cell 76:263-274). In vitro phosphorylation of Erk1/2 has been demonstrated by recombinant murine Ick, and thus provides alternative routes to Erk1/2 activation (Veillette et al., 1988, Cell 55:301-308; Ettehadieh et al., 1992, Science 255:853-855). Although the details of PKC- and p56Lck-mediated pathways of Erk1/2 activation are not well defined, participation of Erk1/2 in cascades of activated protein kinases provides a mechanism of transducing protein-kinase-mediated signaling to the nucleus (Ballif and Blenis, 2001, Cell Growth Differ. 12:397408). These studies together provide evidence that D-type cyclins may function as the link between variety of intracellular signals and the autonomous cell cycle machinery (Sherr, 1993, Cell 73:1059-1065; Sherr, 2000, Cancer Res. 60:3689-3695; Rimerman et al., 2000, J. Biol. Chem. 275:14736-14742). However, in glial cells, both Schwann cells and oligodendrocytes, cell cycle regulation by signaling in response to environmental cues is largely unknown.

In the present study, taking the advantage of the unique capacity of M. leprae to induce proliferation from inside human Schwann cells, the data disclosed herein demonstrate that stimulation by signaling from an intracellular cue alone is sufficient to promote G1/S phase progression of the cell cycle. One such signaling that promotes G1/S phase progression was identified as Lck/Ser-158-mediated Erk1/2 activation that bypasses the classical extracellular mitogen-induced/MEK-dependent Erk1/2 pathway. Therefore, surprisingly, the data disclosed herein demonstrate that Lck/Ser-158 not only serves as a novel cellular activator that directly phosphorylates Erk1/2 in glial cells, but also mediates Erk1/2-induced G1/S phase transition.

The materials and methods relating to this example are now presented.

Isolation, Purification and Characterization of Human Primary Schwann Cells

Human Schwann cells were isolated from peripheral nerve tissues, which comprise nerve roots making up the Cauda equina. Briefly, human Schwann cells were isolated from peripheral nerve tissues, which were obtained from the University of Miami Organ Procurement Organization following the harvest of organs for transplantation. The tissues, consisting of nerve roots making up the Cauda equina, were harvested within 2 hours of aortic clamping and stored at 4° C. in Belzer's (University of Wisconsin) Cold Storage Medium (DuPont, Inc.) until processing, usually within 24 hours of harvest.

Cauda equina roots were teased apart and minced into fragments. These fragments were incubated overnight at 37° C. and 6% CO₂ in Dulbecco's Modified Eagle Medium (DMEM; GIBCO/InVitrogen, Grand Island, N.Y.) containing heat-inactivated 10% (v/v) fetal bovine serum (FBS; Hyclone, Logan, Utah), 0.5 mg/ml collagenase type I (Worthington Biochemical Corp., Lakewood, N.J.) and 1 mg/ml dispase II (Roche Molecular Biochemicals, Indianapolis, Ind.). The resulting cell suspensions were rinsed 3 times with Leibovitz's LI 5 medium, and plated onto uncoated tissue culture-treated dishes in DMEM with 10% FBS for 2 days at 37° C. and 6% CO₂. Under these conditions, most endothelial cells, fibroblasts and macrophages adhered, while leaving the Schwann cells in suspension. The Schwann cells were then plated onto laminin-coated dishes (GIBCO/InVitrogen; 50 μg/100 mm dish) in Medium A, consisting of DMEM, 10% FBS, 2 nM forskolin (Sigma, St. Louis, Mo.) and 10 nM recombinant heregulin peptide 177-244 (Genentech, South San Francisco, Calif.) at a density of 500,000 cells/100 mm dish. When the cells became confluent they were re-suspended by rinsing in Ca⁺⁺ and Mg⁺⁺-free Hank's Balanced Salt Solution (CMFHBSS) and brief (5 minutes at room temperature) exposure to a solution of 0.05% trypsin and 0.02% EDTA in CMFHBSS. Re-suspended cells were rinsed in L-15/10% FBS and re-plated in Medium A into new laminin coated dishes at a density of about 500,000 cells/100 mm dish. The cultures were again allowed to reach confluency. Schwann cells harvested from such cultures were designated Passage 1 or “P1”. These P1 cells were further purified by FACSVantage dual-laser flow cytometer (Beckton-Dickinson, San Jose, Calif.) using p75 MAb as a specific marker for non-myelinating Schwann cells as described in Morrissey et al., 1995, J. Neurobiol. 28:190-201). Using this protocol, highly purified Schwann cell populations were obtained from each human donor. These Schwann cells were finally seeded on collagen-coated flasks (BD BioScience, Bedford, Mass.) and propagated in larger scales. Schwann cells used in the experiments described herein were harvested at P3-P4. The purity of final Schwann cell preparation prior to infection was 98-100% as determined by p75 and S100 immunoreactivity. Functional properties of these Schwann cells at the level of M. leprae invasion demonstrated that all Schwann cell preparations were highly effective for invasion by M. leprae regardless of donor variation.

Other Primaryy Cells and Cell Lines

The breast adenocarcinoma epithelial cell line SKBR-3, mouse fibroblast cell line NIH/3T3, and human neuroblastoma cell line SK-N-AS were obtained from American Type Culture Collection (ATCC, Manassas, Va.) and were propagated according to the protocols from ATCC. SV40-transformed human Schwann cell line used in this study has been described previously (Rambukkana et al., 1998, Science 282:2076-2079).

M. leprae Infection of Primary Human Schwann Cells

Viable M. leprae grown in vivo were derived from the footpads of athymic nu/nu mice as reported previously (Truman and Krahenbuhl, 2001, Int. J. Lepr. Other Mycobact. Dis. 69:674-681). Preparation and characterization of irradiated M. leprae were also described previously (Adams et al., 2000, Int. J. Lepr. Other Mycobact. Dis. 68:1-10). Freshly harvested, viable or irradiated Thai-53 isolate of M. leprae were obtained from the National Hansen's Disease Programs Laboratory, Baton Rouge, La.

M. leprae infection was carried out by inoculation of 1×10⁸ bacteria/ml in approximately 40 to about 50% confluent purified human primary Schwann cells (passage # 3) grown on collagen-coated flasks (BD BioScience).

Schwann cells from at least 5 different human donors were used for infection. The procedure used for M. leprae infection was similar to that described previously (Rambukkana et al., 2002, Science 296:927-931), and infected human Schwann cells were maintained at 33° C. in humidified CO₂ incubator. Infected cultures were fed with Schwann cell media every 2 days and maintained for up to about 30-40 days.

Genearray and Real-Time Quantitative RT-PCR (TagMan) Analyses

Human genome Affymetrix genechips (Santa Clara, Calif.) were used to determine differential cell cycle gene expression of human primary Schwann cells in response to intracellular M. leprae. For real-time RT-PCR, primers and fluorescently labeled probes from the coding regions of human cell cycle regulators were designed using Vector NTI software.

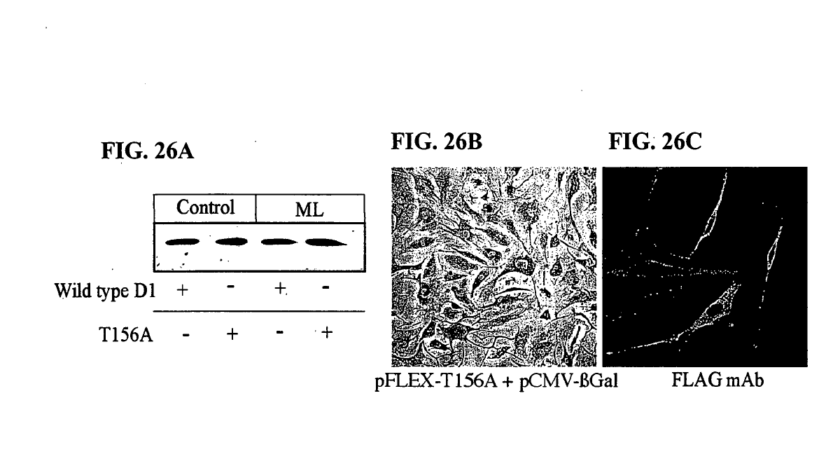

Experimental details of genechip analysis and real-time RCR are as follows. Human genome Affymetrix genechips were used to determine differential cell cycle gene expression of human primary Schwann cells in response to intracellular M. leprae. Total cellular RNA was isolated from M. leprae-infected and non-infected-Schwann cells at day 3, 7, 15, and 30 days using RNeasy spin columns (Qiagen, Valencia, Calif.). Briefly, the total RNA (5 μg) was reverse-transcribed to cDNA and double-stranded cDNA synthesis was performed as described in the Expression Analysis Technical Manual provided by the manufacturer (Affymetrix, Santa Clara, Calif.). The cRNA reactions were performed using the BioArray High-Yield transcript labeling kit (Enzo Diagnostic Inc., Farmingdale, N.Y.). 15 μg of labeled cRNA was fragmented for 35 minutes at 94° C. using fragmentation buffer (200 mM Tris-acetate, pH 8.1, 500 mM potassium acetate, 150 mM magnesium acetate). Affymetrix Test3 arrays and human genome U95Av2 arrays were probed, hybridized, stained and washed according to the manufacturer's instructions. Expression analysis of the differentially expressed genes was performed with the Affymetrix Microarray Suite 5.0 software to obtain absence/presence calls, fold differences and significance (p values). Differentially expressed genes of the M. leprae infected Schwann cells were then clustered according to functional annotations using the Data Mining Tool 3.0 software (Affymetrix) by searching the GeneBank, UniGene and LocusLink databases through the NETAFFX portal (Affymetrix). Clustered genes were also filtered with a 2-fold difference and p<0.01 filter and the genes that satisfied these criteria are shown in FIGS. 26B through 26D.

Antibodies

Primary rabbit polyclonal antibodies used in this study included anti-phospho-Erk1/2 (Thr202/Tyr204), anti-phospho-MEK1/2 (Ser217/221), anti-phospho-Elk1 (Ser383), anti-phospho-GSK-3β, anti-phospho-β-catenin, anti-phospho-Akt, anti-phospho-PKC-alpha-betaII/βII (Thr638/641), anti-phospho-PKC∂ (Thr505), anti-phospho-PKC∂ (Ser643), anti-phospho-PKD/PKCμ (Ser744/748), anti-phospho-PKD/PKCμ (Ser916), anti-phospho-PKCθ (Thr538), anti-phospho-PKCζ/λ (Thr410/403), anti-phospho-Rb1, anti-phospho-p38, anti-phospho-JNK, anti-phospho-Rb1 and anti-phospho-Elk (Cell Signaling Technology, Beverly, Mass.); anti-phospho-Lck (Ser158), anti-total-Lck (Biosource, Camarillo, Calif.); anti-human Cyclin D1 and anti-human p21 and anti-human Caveolin-1 (Santa Cruz, Calif.); anti-S100 (Dako, Carpinton, Calif.). Primary mouse monoclonal antibodies used were anti-human Cyclin B, anti-human cyclin A, anti-human Rb1, anti-human Rb2, anti-human Cdk4 (B&D Biosciences, Lexington, Ky.); anti-BrdU (Roche Molecular Biochemicals, Indianapolis, Ind.); anti-p75 (a gift of Dr. P. M. Wood, University of Miami, F1); anti β-Actin, anti-human vinculin, and anti-FLAG (Sigma); anti-BrdU (Roche Molecular Biochemicals); anti-M. leprae PGL-1 and anti-p75 were provided by the Royal Tropical Institute, Amsterdam, and the University of Miami, Fla., respectively.

Western Blot and Other Biochemical Analysis

Total protein extracts were prepared by harvesting M. leprae infected human Schwann cells in cell lysis buffer and non-infected human Schwann cells in nuclear extracting buffer. Lysates were cleared by centrifugation and the protein content was determined according to the BCA method (PIERCE Chemical, Rockford Ill.). 40%1 g of total cellular extract proteins were separated by sodium dodecyl sulfate-polyacrylamide gel electrophoresis (SDS-PAGE) and transferred to nitrocellulose membranes (Schleicher & Schuell). The blots were probed with antibodies and the bound antibodies were detected using species-specific secondary antibodies coupled to horse radish peroxidase (HRP) (Cell Signaling Technology) followed by enhanced chemiluminescence development (PIERCE Chemical, Rockford Ill.). Protein expression intensity was normalized to β-actin expression and quantified with a densitometer (Alpha Innotech Corporation, San Leandro, Calif.) using the Alpha Imager 2200 v.5.5 software. For preparation of nuclear and cytoplasmic extracts, cells were incubated in a buffer containing 10 mM HEPES pH 7.5, 60 mM KCl, 1 mM EDTA, 0.1% NP-40, 1 mM dithiothreitol (DTT), 1 mM PMSF and protease inhibitors cocktail for 10 minutes on ice. Lysates were then centrifuged for 10 minutes and the supernatant, which comprised cytoplasmic extract, was collected. The pellet was re-suspended in a buffer containing 50 mM Tris-HCl pH 7.5, 150 mM NaCl, 1% sodium lauryl sarcosine, 1% NP-40, 0.1% SDS, 1 mM EDTA and protease inhibitors cocktail. After a brief sonication, the lysates were centrifuged for 10 minutes and the supernatant, which consists of nuclear extract, was collected. The protein content in both extracts was determined by the BCA method.

Immunofluorescence

Infected and control Schwann cells were trypsinized and seeded onto collagen-coated coverslips (BD Biosciences, Bedford, Mass.). The cells were washed twice with PBS and fixed with cold methanol for 10 minutes. After blocking and incubation with the relevant primary antibodies, cells were washed and incubated with affinity purified, rhodamine-conjugated goat anti-rabbit IgG, and/or fluorescein-conjugated goat anti-mouse IgG (Chemicon International, Temecula, Calif.). Images were captured with Nikon Eclipse 2100 and Zeiss LSM 510 confocal microscopes.

Synchronization of Primary Schwann Cell and Kinase Inhibition

Human Schwann cells that had been cultured for 30 days with and without M. leprae were synchronized for 48 hours and then released to re-enter the cell cycle by addition of 10% serum. Cytoplasmic and nuclear extracts of these synchronized cells were prepared before and after addition of serum at different time points (9 to 18 hours after release). For Erk1/2 and PI3-k inhibition, pre-evaluated concentrations of specific MEK1/2 inhibitor UO126 (15 μM) and PD098059 (15 μM), pI3-k inhibitor LY294002 (15 μM) (Cell Signaling Technology), SOS inhibitor (10 μg) (Upstate Biotechnology, Lake Placid N.Y.) were used. For PKC and lck inhibition, pan-PKC inhibitor bisindolylmaleimide-I (20 nM) and Lck inhibitor PP2 (10 μM) (Calbiochem-Novabiochem Corporation, CA), respectively, were added to the culture medium during synchronization, and cytoplasmic and nuclear extracts were prepared as described above and were immunoblotted with antibodies to various phospho-specific signaling antibodies. None of the inhibitors produced any apoptosis or Schwann cell death under these conditions.

To examine if the addition of exogenous Heregulin-β1 can induce Lck/Ser-158 phosphorylation, human Schwann cells were serum-starved for 48 hours and then incubated with 10 nM P1-Heregulin for 30 minutes. Subsequently, total protein lysates were prepared and the phosphorylation state of Erk1/2, PKC-alpha-betaII/βII and Lck-Ser158 were examined using phospho-specific antibodies.

Kinase Activity Assays

MAP kinase activity was determined by the capacity of p44/42 (Erk1/2) to phosphorylate Elk-1 in vitro using p44/42 activity assay kit according to manufacturer's instructions (Cell Signaling Technology, Beverly, Mass.). Briefly, phosphorylated p44/42 was immunoprecipitated from M. leprae-infected and non-infected human Schwann cell lysates with an immobilized monoclonal phospho-antibody to p44/42 Map kinase (Cell Signaling Technology). The resulting immunoprecipitate containing active MAP kinase was incubated with an Elk-1 fusion protein in the presence of 50 μM ATP and kinase buffer (25 mM Tris pH 7.5, 5 mM β-glycerolphosphate, 2 mM DTT, 0.1 mM Na₃VO₄, 10 mM MgCl₂). Immunoprecipitated active Erk1/2 was then allowed to phosphorylate Elk-1. Phosphorylation of Elk-1 at Ser 383, a major phosphorylation site of Erk1/2, was measured by western blotting using a phospho-Elk-1 (Ser 383) antibody (Cell Signaling Technology).

Using a similar approach, the capacity of Lck to phosphorylate Erk2 was assessed using anti-phospho Lck (Ser 158) antibody (Biosource) to immunoprecipitate the phosphorylated endogenous Lck from the lysates of M. leprae-infected and control human Schwann cells and recombinant inactive Erk2 (Cell Signaling Technology). In vitro phosphorylation of Erk2 was assessed using anti-phospho-Erk1/2 (Thr202/Tyr204) antibody (Cell Signaling Technology) and/or phospho-Tyr antibody (B&D Biosciences). Isotype control rabbit antibodies (Sigma) were used in immunoprecipitations as negative controls.

Cell Cycle FACS Analysis and Transformation Assay

Human primary Schwann cells, that had been cultured for 15 and 30 days with and without M. leprae were trypsinized and adjusted to equal cell number. For the experiments with kinase inhibitors, synchronized infected and control cells were treated with pre-evaluated concentrations of various inhibitors as described previously elsewhere herein, and with different combinations of inhibitors before being processed for cell cycle FACS (Lemoine and Marriott, 2001, J. Biol. Chem. 276:31851-31857). To assess the effect of Lck inhibitor on heregulin-induced cell cycle progression, synchronized Schwann cells were treated with heregulin for 30 minutes in the presence and absence of Lck inhibitor. Cells were then washed with PBS, fixed with 85% ethanol overnight at 40° C. and suspended in 1 ml PBS containing 1% FBS, RNase A (250 μg/ml; Roche Molecular Biochemicals) and 100 μg/ml Propidium Iodide (Sigma) for 30 minutes at 37° C. Stained cells were washed twice with PBS and subjected to FACS analysis on a Becton Dickinson FACScan cytometer (Ruhl et al., 1999, J. Biol. Chem. 274: 34361-34368). The cell cycle profile was analyzed with CellQuest software (Becton & Dickinson).

Transformation Assay

Transformation assays were performed according to an established protocol (Lee et al., 2000, Mol. Cell. Biol. 20:672-683). Briefly, M. leprae-infected and non-infected-Schwann cells and SK-BR-3 breast cancer cells were trypsinized and the cells were seeded onto 100-mm-diameter poly-L-lysine-coated dishes. The culture media were changed twice weekly for 21 days. Cells were washed twice with PBS, fixed with 10% acetic acid for 10 minutes and then stained with 0.4% crystal violet (Sigma) in 10% ethanol for 10 minutes. The dishes were washed, inverted and dried at room temperature. Plaque formation was evaluated by colony counting under an inverted microscopy.

Transcription Factor Array

To identify potential transcription factors associated with the regulation of human Schwann cell cycle during M. leprae infection, the TranSignal Protein/DNA Array (Panomics Inc., CA) was used. Nuclear extracts from control and infected Schwann cells were prepared according to manufacturer's instructions. Nuclear extracts were incubated with a set of biotin-labeled DNA binding oligonucleotides (TranSignal Probe Mix, Panomics, Inc.) to allow the formation of DNA/protein complexes, which were then hybridized to the TranSignal Arrays spotted with different human consensus-binding sequences, each corresponding to a specific transcription factor. Binding was detected by incubating the arrays with Streptavidin-conjugated HRP, and normalization and analysis were performed with a densitometer (Alpha Innotech Corporation) using the Alpha Imager 2200/5.5 software.

Electrophoretic Mobility Shift Assays (EMSA)

Biotinylated and unlabeled (cold) double-stranded oligonucleotides containing the consensus binding site of E2F-1 (5′-ATTTAAGTTTCGCGCCCTTTCTCAA-3′), were purchased (Panomics, Inc., CA). Nuclear extracts of Schwann cells were prepared as described above. Equal amounts (5 μg) of infected and control Schwann cell nuclear extracts were mixed with 2 ng of biotin-labeled probe in 10 μl of binding buffer containing 2 μg of poly(dI-dC) and incubated at 20° C. for 30 minutes. For competition experiments, 10-fold of unlabeled (cold) oligonucleotide was included in the reaction mixture. Protein-DNA complexes were separated on non-denaturing 4% polyacrylamide gels and transferred to Biodyne B membranes (Pal Gelman Laboratories). Bound proteins were detected using HRP-conjugated Streptavidin with enhanced chemiluminescence (Pierce).

Transient Transfection Experiments

For transient expression of cyclin D1 dominant negative mutant the pFLEX-D1 and the pFLEX-T156A constructs (Diehl and Sherr, 1997, Mol. Cell. Biol. 17:7362-7374) were introduced into semi-confluent (about 60%) 60-mm-diameter dishes of M. leprae-infected and non-infected Schwann cells by the calcium phosphate method according to manufacturer's instructions (Promega). For Erk1 inhibition, pcDNA1Neo-HA-T192A Erk1 dominant negative mutant was used (Pages et al., 1993, Proc. Natl. Acad. Sci. USA 90:8319-8323). For reporter expression the pCMV•SPORT-βgal vector (Invitrogen Life Technologies) was used. To determine transfection efficiency, transfected cells were counted on a light microscope over 10 contiguous fields and the average percentage ±STDEV of the β-galactosidase positive cells was assessed. Transfections were terminated at 48 hours and the cells were assayed for transfection efficiencies (β-galactosidase staining) and processed for cell-cycle FACS analysis as described above.

The results relating to the instant example are now discussed.

Intracellular M. leprae Induce Continuous Proliferation of Human Schwann Cells

To recapitulate the fate of human non-myelinating Schwann cells in response to long-term intracellular residence of M. leprae in vivo, Schwann cells were isolated from peripheral nerves from different human donors, and each isolation was further purified to homogeneity by FACS using MAb against neurotrophin receptor p75 as a marker for non-myelinating Schwann cells (FIG. 1A). Cultured cells (passage #3) were also positive for S-100, p75 and GAP43. The latter two markers are expressed by mature non-myelinating Schwann cells but not by myelinating Schwann cells (Curtis et al., 1992, J. Cell. Biol. 116:1455-1464; Bentley and Lee, 2000, J. Neurosci. 20:7706-7715), (FIG. 1B). Since these Schwann cells efficiently engulfed M. leprae (FIGS. 1C and 1D), they are also functionally similar to non-myelinating phenotypes (Rambukkana et al., 2002, Science 296:927-931). In all experiments, cells from early passages (passage number 3 or 4) were used, as they closely mimic the in vivo-like human Schwann cell environment.

Intracellular bacteria were detected in about greater than 95% of Schwann cells (ranging from about 5 to 40 bacteria per cells) by MAb specific for phenolic glycolipid (PGL-1) of M. leprae cell wall. To study the Schwann cell response to long-term intracellular residence of M. leprae, these infected cultures were further incubated for 3, 7, 15 and 30 days (40 days in some cases). Despite the significant Schwann cell growth during these incubation periods, infected cells retained numerous intracellular M. leprae after 15 and 30 days (FIGS. 1C and 1D).

Strikingly, human Schwann cells that harbored M. leprae showed no apoptosis or cytopathic effects as revealed by nuclear labeling and electron microscopy (FIG. 1D). Both TUNNEL assays and immunoblotting of lysates of infected cells with antibodies to specific apoptotic markers, Caspase-3, Caspase-9 and poly (ADP-ribose) polymerase (PARP), showed no difference from controls even after 30 and 40 days of infection, whereas Schwann cells treated with Camptothecin induced the expression of these apoptotic markers within 48 hours (FIG. 1E). In contrast, M. leprae infected Schwann cells exhibited a significant increase in 5-bromo-2-deoxyuridine (BrdU) positive cells and a proliferative S phase population as determined by FACS (FIG. 1F and FIG. 1G). A highly significant proliferation index (about 40%; p<0.001) was observed 30 days after infection (FIG. 1G). Since both confocal and electron microscopy of 30-day infected cells showed the localization of M. leprae inside the cytoplasm, but not on or outside the cells (FIG. 1D), it was concluded that Schwann cell proliferation is caused by intracellular M. leprae.

Genearrays to Identify Target Human Schwann Cell-Cycle Genes in Response to Intracellular M. leprae: Role of G1 Phase Regulators

Since cell proliferation results as a completion of cell cycle progression, the ability of intracellular M. leprae to modulate human Schwann cell cycle machinery was examined. Affymetrix human genechip arrays was used to identify key target cell cycle genes in human Schwann cells that are differentially regulated in response to intracellular M. leprae. RNA from infected and non-infected human Schwann cells were hybridized to human genechips, and differentially expressed genes were analyzed by using the Affymetrix software as described elsewhere herein.

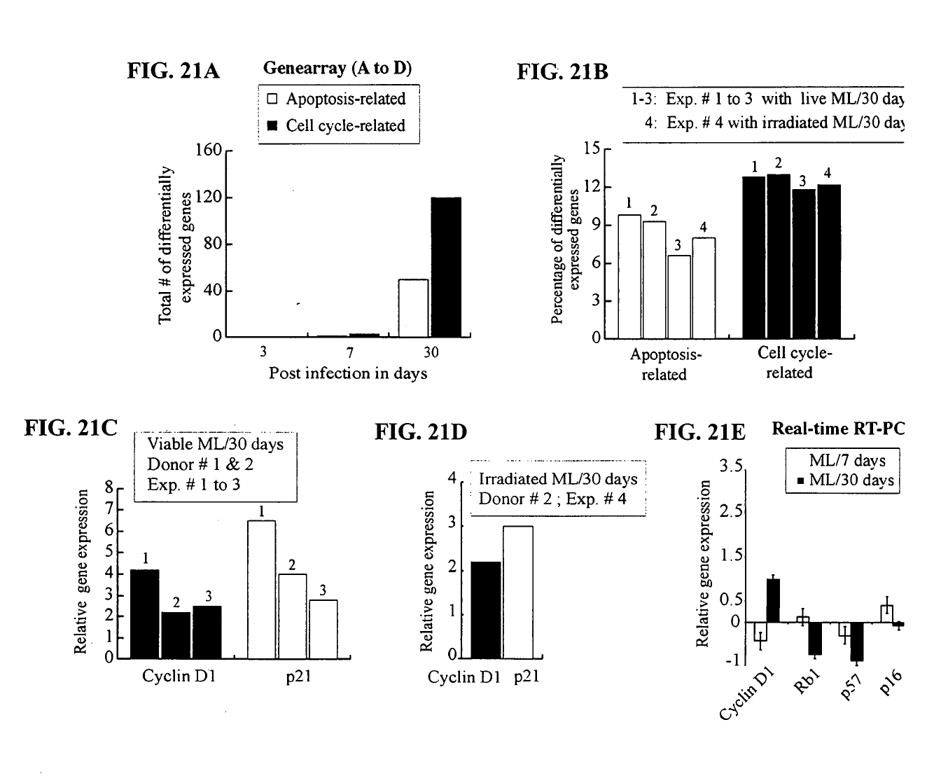

First, the differentially expressed cell cycle-related genes were compared at 3, 7 and 30 days after infection, and a broader gene expression pattern was observed in that day 30 post-infection population (FIG. 21A). These data indicate that intracellular M. leprae exert significant activation of cell cycle genes after long-term residence within Schwann cells. Therefore, the studies focused on the day 30 post-infection population, and genearray experiments at this time point were repeated three times (total 4 genearray experiments) with Schwann cells from two different human donors. Differentially expressed genes were clustered according to functional annotations. Selected human Schwann cell genes with known functional significance to cell proliferation and apoptosis are listed in the Table 1. All other differentially expressed apoptosis- and cell cycle-related genes are listed in Table 2. FIG. 21B shows the percentage of differentially expressed genes of these two categories in four different genechip experiments (Experiments 1-4); percentage was calculated on the basis of the total number of spotted genes onto the chip. As can be seen, the percentage of expressed genes in individual experiments (Experiments 1-4) was almost identical, demonstrating high reproducibility of the genearray experiments and the confirmation of specific gene expression by intracellular M. leprae (FIG. 21B; Table 1).

To determine the reliability of gene expression, the differentially expressed cell cycle genes were clustered with Data Mining Tool 3.0 Affymetrix software to satisfy the following three criteria: (i) relative gene expression by 2-fold, (ii) significant value of p<0.001 from baseline level, and (iii) expression of individual genes in all four experiments. According to these criteria, cyclin D1 and p21 were identified as the major cell cycle regulatory genes in all four genearray experiments. However, it is important to note that numerous other differentially expressed genes, although about less than 2-fold, were also statistically significant compared with baseline levels in all four genearray experiments (p value range from 0.01 to 0.0001; Table 1), and thus these data indicate the genes were likely to be effective functionally. Mean fold-increase of cyclin D1 and p21 genes in response to viable M. leprae (from three individual experiments with two different human donors) after 30 days of infection is shown in FIG. 21C. These data indicate the possible role of cyclin D1 and p21 as major regulators in Schwann cell cycle progression in response to intracellular M. leprae.

To further confirm genearray data, Real-Time Quantitative RT-PCR (TaqMan) analysis was performed. Infected and non-infected human Schwann cells were maintained for 7 and 30 days and total RNA was isolated to perform the Real-Time PCR using specific primer/probe combinations for key cell cycle regulators. This analysis confirmed the genearray data that the cyclin D1 and p21 genes were significantly upregulated by intracellular M. leprae after 30 days of infection. In addition, genes for G1 phase checkpoint regulator Rb1 were identified, and negative regulators p57 and p16 were down regulated at day 30 (FIG. 21E).

Role of M. leprae Cell Wall Components in Schwann Cell Cycle Gene Activation

To examine the requirement of viability of intracellular M. leprae in Schwann cell gene expression, human Schwann cells were infected with irradiated M. leprae for 30 days and maintained under similar conditions as viable M. leprae. Irradiated M. leprae were found inside 30 day-infected Schwann cells similar to live M. leprae as demonstrated in FIGS. 1C-1D. Strikingly, the gene expression was not affected by the viability of M. leprae, since almost the identical gene profile was obtained from Schwann cells infected with irradiated M. leprae (Table 1; FIGS. 1B-1D). Although irradiated M. leprae are metabolically inactive (Adams et al., 2000, Int. J. Lepr. Other Mycobact. Dis. 68:1-10), their cell wall components remain intact and possess biological activity; the capacity of irradiated M. leprae to invade non-myelinated Schwann cells and to induce demyelination in myelinated Schwann cells is as effective as viable M. leprae (Ng et al., 2000, Cell 103:511-524; Rambukkana et al., 2002, Science 296:927-931). After setting a cutoff value of 2-fold for gene expression, it was demonstrated that irradiated M. leprae had the ability to induce cyclin D1 and p21 genes in a similar manner as viable M. leprae (FIGS. 21C and 21D). These data suggest that Schwann cell gene activation by intracellular M. leprae is largely mediated by the components of the bacterial cell wall.

Intracellular M. leprae Induce Anti-Apoptotic Responses in Human Schwann Cells

Although high percentage of apoptotic-related genes was detected in response to viable and irradiated M. leprae (Table 1; FIGS. 21A and 21B; Table 2), infected Schwann cells showed no apoptosis or cell death (FIGS. 1D and 1E). This is, without wishing to be bound by any particular theory, likely due to the fact that most of these apoptotic genes, particularly those which play a direct role in cell death process, such as caspases, AIF, Fas, GAD45, GADD34 and Bax, are down regulated, and the up-regulated genes include some of the key anti-apoptotic genes, such as Bcl-2 and BCL-xL (Table 1). Thus, the overall effect of such gene expression may prevent Schwann cell apoptosis even after long-term bacterial residence. This was further confirmed at the protein level, since antibodies to caspases and PARP did not show any positive reaction with immunoblots of 30 day-infected Schwann cells (FIG. 1E).

Intracellular M. leprae Modulate Key G1 Phase Cell Cycle Proteins: Role of Cyclin D1

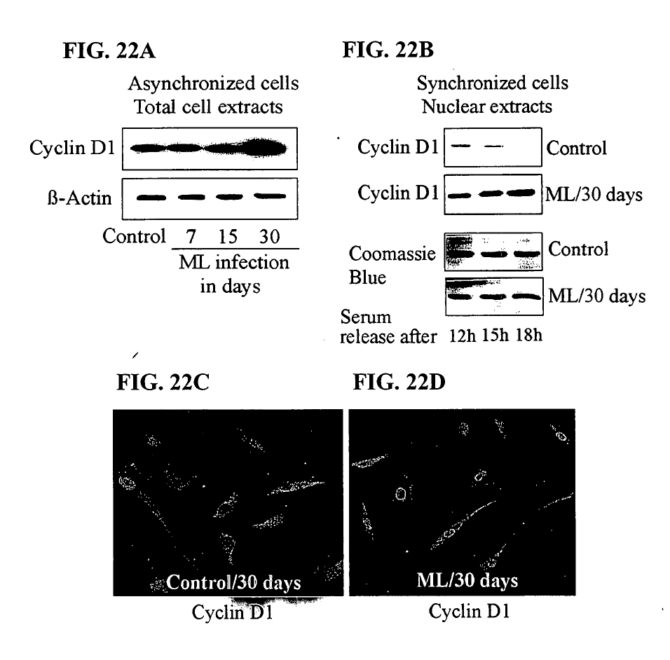

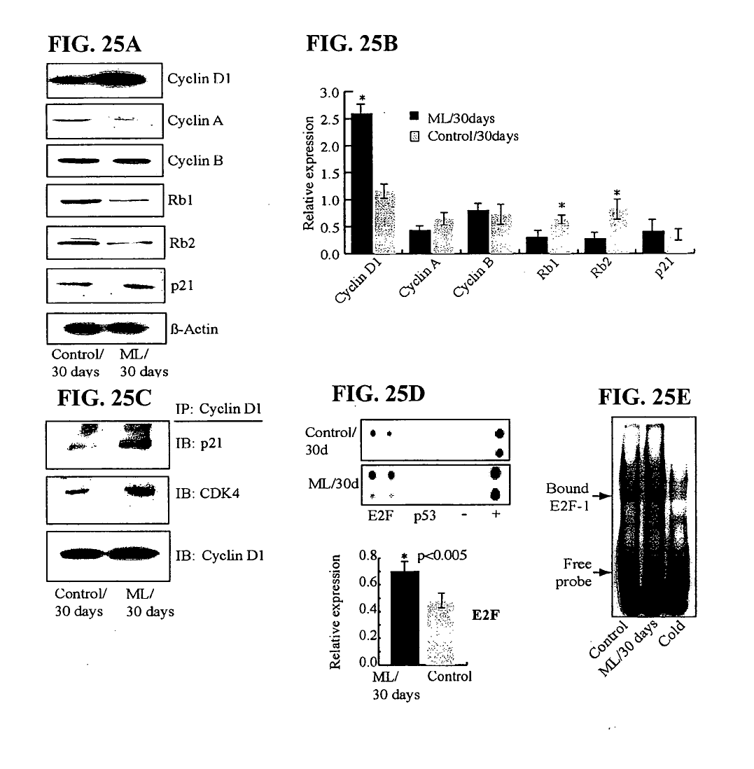

To examine whether the observed M. leprae-induced gene expression pattern corresponds to cell cycle protein profiles, total protein extracts from asynchronous Schwann cell cultures infected with M. leprae at 7, 15 and 30 days were analyzed by immunoblotting using specific antibodies to various cell cycle regulators. Significant up-regulation of cell cycle regulators at protein level was observed after 30 days of infection, and cyclin D1 protein showed the most significant increase as compared to other proteins (FIG. 22A; FIG. 25). On the other hand, the protein levels of Rb1 (p130) and Rb2 (p107) were considerably decreased and there was no change in Rb phosphorylation (FIG. 25), which usually occur in response to extracellular mitogens (Nevins, 1992 Science 258:424-429). On the other hand, the protein levels of Rb1 (p 130) and Rb2 (p107) were considerably decreased, whereas no major change was observed in cyclin A, cyclin B and p21, at day 30 (FIGS. 25A and 25B). However, increased p21 was detected only when lysates were immunoprecipitated with anti-cyclin D1 as discussed elsewhere herein (FIG. 25C).

To determine whether cyclin D1 assembles with p21 and CDK4 in M. leprae-infected Schwann cells, total Schwann cell lysates were immunoprecipitated with cyclin D1 antibody, and then immunoprecipitated proteins were immunoblotted using anti-CDK4 and anti-p21 antibodies. Both CDK4 and p21 were detected in infected Schwann cells in cyclin D1 precipitates in high proportions as compared to controls (FIG. 25C). The studies focused on cyclin D1 because of the critical role of cyclin D1 in G1/S phase transition in mouse Schwann cells (Kim et al., 2000, Neuron 26:405-416).

Next, the role of intracellular M. leprae to regulate nuclear accumulation of cyclin D1, which subsequently leads to cell cycle progression, was examined. After 30 days, both infected and control Schwann cell cultures were synchronized for 48 hours and then released into the cell cycle by the addition of serum, a well established method for studying the kinetics of cell cycle progression (Pardee, 1989, Science 246:603-608). Nuclear extracts from synchronized Schwann cell cultures were prepared before and after addition of serum at various time points and analyzed by immunoblotting using cyclin D1 antibody. A significant increase in nuclear cyclin D1 was observed only in infected cells upon serum addition, particularly after 12 to 18 hours (FIG. 22B), the time points corresponding to mid- to late-G1 phase (Kim et al., 2000, Neuron 26:405-416). immunofluorescence experiments under similar conditions further showed that the number of cells with nuclear labeling of cyclin D1 was dramatically increased in infected Schwann cells 15 hours after serum release (FIGS. 22C and 22D). Also, the S-phase population of infected cells was increased 15 hours and 18 hours after serum release. Without wishing to be bound by any particular theory, a possible explanation for these results is that intracellular M. leprae continuously activate signaling in synchronized Schwann cells and thereby promote increased accumulation of nuclear cyclin D1 and cell cycle progression upon serum release.

Intracellular M. leprae Accelerated G1 Phase Progression and Increased the Total Number of Schwann Cells

Given the ability of intracellular M. leprae to modulate the key cell cycle regulators, particularly the nuclear accumulation of cyclin D1, the role of M. leprae to accelerate cell cycle progression in infected Schwann cells was examined. Asynchronous Schwann cell cultures 15 and 30 days after infection were subjected to cell cycle FACS analysis. The cell cycle profile of Propidium Iodide-treated Schwann cells (analyzed with collects software as provided by the manufacturer) revealed that the total cell count in G1 phase of 30 day-infected Schwann cells was significantly reduced as compared to controls whereas infected cells particularly in G2 phase were dramatically increased (FIG. 7A). This suggests that intracellular M. leprae accelerated G1 phase progression with earlier S phase entry, and that significant number of cells have entered G2 phase. However, this accelerated cell cycle progression with reduced time allowed for G1 to S phase transition did not affect the size of infected cells, which were indistinguishable from uninfected cells. This is in contrast to mammalian cells infected with certain oncogenic viruses that reduce the size of cells as they accelerate the G1 progression and ultimately cause cellular transformation in primary cultures (Lemoine and Marriott, 2001, J. Biol. Chem. 276:31851-31857). Importantly, in contrast to M. leprae-infected cells, virally infected cells do not increase G2 phase population (Lemoine and Marriott, 2001, J. Biol. Chem. 276:31851-31857). This suggests that regulation of Schwann cell cycle by intracellular M. leprae is different from those oncogenic viruses.

To further delineate the direct role of cyclin D1 in M. leprae-induced cell cycle progression, 30 day-infected cell population and control primary human Schwann cells were transfected with a dominant negative cyclin D1 mutant (T156A with N-terminal FLAG) that has previously been shown to form catalytically inactive cytoplasmic complexes with CDK4 (Diehl and Sherr, 1997, Mol. Cell. Biol. 17:7362-7374). T156A expression is known to prevent nuclear accumulation of cyclin D1 and induce cell cycle arrest by blocking G1 phase progression (Diehl and Sherr, 1997, Mol. Cell. Biol. 17:7362-7374). The overexpression of the cyclin D1 dominant negative mutant in 30 day-infected Schwann cells reversed the effects of intracellular M. leprae on Schwann cell cycle progression (FIG. 7B and FIG. 26).

Transfection of both infected and control Schwann cells with T156A-FLAG or wild type D1-FLAG showed an equal expression pattern of T156A-FLAG or D1-FLAG as analyzed by anti-FLAG antibody (FIG. 26A). When infected Schwann cells transfected with T156A-FLAG were co-transfected with a reporter vector expressing β-galactosidase, 60% transfection efficiency was obtained as a mean value from four independent experiments (FIG. 26B). Labeling of M. leprae-infected/T156A transfected Schwann cells with anti-FLAG antibody 48 hours post-transfection demonstrated that the T156A mutant was detectably expressed exclusively in the cytoplasm of transfected Schwann cells (FIG. 26C).

G1 population was significantly increased with a concomitant decrease in S and G2 populations in 30 day-infected/T156A transfected Schwann cells as compared to 30 day-infected/non-transfected Schwann cells. On the other hand, carrier vector alone or a reporter β-galactosidase vector exhibited no effect on M. leprae-induced G1 phase shortening (FIG. 7B and FIG. 26), suggesting that functional blockade of cyclin D1 is specific, and is sufficient to abolish cell cycle progression stimulated by intracellular M. leprae.

Since cell division-cycle should result in cell propagation as the final outcome, it was next determined the total Schwann cell count during the course of infection. When equal numbers of cells were seeded and propagated with and without M. leprae under similar experimental conditions as above, it was observed that the total cell count of 15 day- and 30 day-infected cultures, but not 7 day-infected cultures, was significantly increased (p<0.0001) as compared to controls (FIG. 7C). It should be noted that although the total number of infected Schwann cells were increased by about 20-fold (mean value from 4 experiments) during incubation time, approximately greater than 90% Schwann cells at 30 days still carried numerous intracellular M. leprae. (FIG. 1D). Thus, without wishing to be bound to any particular theory, it is likely that the observed cell cycle gene regulation and progression are due to Schwann cell response to long-term residence of intracellular M. leprae.

Decreased Rb Expression and Increased E2F-DNA Binding Capacity in M. leprae-Infected Schwann Cells

A key physiologic substrate of the cyclin D-dependent kinases is the Rb tumor suppressor protein (Nevins, 1992, Science 258:424-429; Herrera et al., 1996, Mol. Cell. Biol. 16:2402-2407). Phosphorylation of Rb abrogates its activity as a transcriptional repressor of E2F transcription factors, which eventually modulate the proliferation-associated genes (Nevins, 1992). It was examined whether the decrease in total Rb (phosphorylation-independent) both at the gene and protein levels in infected cells facilitates E2F-DNA binding capacity. Using transcription factor arrays (Protein/DNA arrays) and electromobility gel shift assays (EMSA) with E2F-1 consensus oligonucleotide, an increased E2F-1 binding was detected in M. leprae-infected Schwann cells as compared to non-infected cells (FIGS. 25D and 25E). In contrast, infected cells showed no detectable binding activity with p53 under similar experimental conditions, suggesting that intracellular M. leprae do not activate p53-dependent checkpoint responses that lead to either cell cycle arrest or apoptosis (Vogelstein et al., 2000, Nature 408:307-310). On the other hand, elevated E2F1 level in infected Schwann cells suggests the release of E2F1 from Rb complex and its presence as free E2F. Since the expression of free E2F is indicative of cells proceeding through cell cycle (Nevins, 1992, Science 258:424-429), E2F could facilitate intracellular M. leprae to accelerate G1 progression.

Intracellular M. leprae do not Cause Cellular Transformation

Because intracellular M. leprae maintain a sustained up-regulation of cyclin D1 and cell proliferation, it was examined whether infected primary Schwann cells undergo transformation. A standard plaque-forming assay was performed (Lee et al., 2000, Mol. Cell. Biol. 20:672-683) with 30 day-infected Schwann cell and control cultures, and compared with a well-characterized human breast cancer cell line SK-BR-3 (ATCC) and human neuroblastoma cancer cell line SK-N-AS from neural tissues as positive controls for plaque formation (Lee et al., 2000, Mol. Cell. Biol. 20:672-683). Whereas both SK-BR-3 and SK-N-AS cells formed numerous plaques, plaque formation was not detected in 30 day-infected Schwann cells, which were identical to non-infected Schwann cells (FIG. 7D).

To further confirm these finding at molecular level, the expression of caveolin-1, a principal component of the caveolae membrane that is directly associated with transformation suppression activity was analyzed (Glenney and Soppet, 1992, Proc. Natl. Acad. Sci. USA 89:10517-10521). Caveolin-1 is specifically downregulated in transformed cells and the loss of caveolin-1 protein expression has been shown to be sufficient in mediating cell transformation (Galbiati et al., 1998, EMBO J. 17:6633-6648). No difference was detected in gene and protein expression pattern of caveolin-1 between infected and uninfected Schwann cells, whereas the expression of caveolin-1 protein was almost undetectable in SKBR-3 cancer cells and SV40-transformed human Schwann cell lines (FIG. 7E). These data provide further evidence that intracellular M. leprae induce cell proliferation without risk of tumorgenesis.

Signaling Pathways Induced by M. leprae from Inside Human Schwann Cells

Abundance of nuclear cyclin D1 and subsequent G1 phase progression of the cell cycle requires the participation of multiple signaling pathways, which include Ras-dependent effectors such as Erk1/2 and PI3-kinase that are normally activated by binding of growth factors and extracellular matrix (ECM) proteins to receptor tyrosine kinase and integrins, respectively (Roovers and Assoian, 2000, Bioessays 22:818-826; Chang et al., 2003, Int. J. Oncol. 22:469-480). To examine the ability of intracellular M. leprae to activate these signaling pathways in the absence of stimuli from extracellular mitogens, 30 day-infected Schwann cells were synchronized for 48 hours by removing the serum and growth factors, and total lysates were immunoblotted with antibodies to various phospho (p)-specific signaling molecules. A significant up-regulation of pErk1/2 and pGSK3β was detected in infected cultures as compared to controls under these conditions. However, it was difficult to detect the phosphorylation of Akt, β-Catenin, p38 MAPK, and JNK in infected cells after synchronization (FIG. 19A shows the data from two human donors). These findings suggest that phosphorylation of Erk1/2 and GSK-3β that regulate cyclin (Diehl et al., 1998, Genes Dev. 12:3499-3511; Amanatullah et al., 2001, Methods Enzymol. 333:116-127) are specifically induced by intracellular M. leprae in the absence of extracellular mitogenic responses.

Intracellular M. leprae Activate Erk1/2 Through MEK1/2- and Pi3K-Independent Signaling Pathway

Without wishing to be bound to any particular theory, if intracellular M. leprae use extracellular mitogen-activated SOS>Ras>Raf>MEK or PI3-k/Akt pathways to phosphorylate Erk1/2 and GSK-3β respectively (for example, by inducing the release of Schwann cell growth factors), pharmacological inhibition of SOS, MEK or PI3-k should block downstream phosphorylation of Erk1/2 and GSK-3β, respectively (Vlahos et al., 1994, J. Biol. Chem. 269:5241-5248; Favata et al., 1998, J. Biol. Chem. 273:18623-18632).

Surprisingly, despite the synchronization and the continuous presence of specific MEK1/2 inhibitor U0126 and PI3-kinase inhibitors LY294002, phosphorylation of Erk1/2 and GSK-3β was not inhibited in infected Schwann cells (FIG. 19B). pErk1/2 and pGSK-3β were also detected with individual inhibitors in infected cells. Moreover, treatment of synchronized Schwann cells with SOS-inhibitory peptide, that abolished the upstream Ras-dependent Erk1/2 signaling, also failed to inhibit the phosphorylation of Erk1/2 in infected Schwann cells (FIG. 19C). This further suggests that Erk1/2 activation is not mediated by upstream receptor-mediated SOS-dependent signaling. The possibility of donor variation was excluded, since Schwann cells purified from 3 different donors provided almost identical results.

Because of the pivotal role of Erk1/2 signaling in cell cycle progression, the studies focused on Erk1/2. Erk1/2 (p44/p42) kinase activity in infected Schwann cells was determined by analyzing the capacity of activated cellular Erk1/2 to phosphorylate transcription factor Elk-1, a major substrate of Erk1/2 (Whitmarsh et al., 1995, Science 269:403-407). It was observed that active Erk1/2 immunoprecipitated from infected synchronized Schwann cells produced significantly higher phosphorylation of Elk-1 as compared to much weaker activity in non-infected Schwann cells (FIG. 19D; left lanes), confirming that the increased dual phosphorylation of Erk1/2 in infected cells (FIG. 19A) reflected elevated Erk1/2 activity.

Strikingly, high level of Erk1/2 (p44/p42) kinase activity was detected in infected cells as compared to controls in the presence of MEK and Pi3-K inhibitors (FIG. 19D; right lanes). This confirms that the M. leprae-induced dual phosphorylation of Erk1 and Erk2 (FIG. 19B; right lanes) indeed reflected increased kinase activity (FIG. 19D; right lanes). It should be noted that although dual phosphorylation of Erk1 and Erk2 was observed in controls (FIG. 19B; left lanes), kinase activity in these cells was almost undetectable (FIG. 19D; left lanes), suggesting that infected Schwann cells are capable of executing the functional effects of Erk1/2 phosphorylation upon translocation to the nucleus.

Since the phosphorylation state of Erk1/2 has been shown to link directly to cyclin D1 nuclear accumulation (Lavoie et al., 1996, J. Biol. Chem. 271:20608-20616), the effect of MEK1/2-independent Erk1/2 signaling on the status of cyclin D1 in infected cells was analyzed. Consistent with up-regulated Erk1/2 phosphorylation, cyclin D1 expression in nuclear extracts of synchronized infected Schwann cells was considerably increased in the presence of MEK and pI3-k inhibitors (FIG. 19E).

Because nuclear accumulation of cyclin D1 is indicative of S-phase entry, the direct role of Erk1/2 in M. leprae-induced S-phase population of Schwann cells was examined. Human Schwann cells that have been infected with M. leprae for 30 days were transiently transfected with a p44mapk dominant negative mutant T192A (Pages et al., 1993, Proc. Natl. Acad. Sci. USA 90:8319-8323) and the cell cycle kinetics were analyzed by FACS as described elsewhere herein. Infected Schwann cells transfected with T192A showed a significant reduction in S-phase population as compared to cells transfected with an empty vector (p<0.0001) (FIG. 19F). In addition, infected Schwann cells transfected with the p44mapk dominant negative mutant exhibited a marked increase in sub-G1 phase population (p<0.0001) (FIG. 19F). This suggests that inhibition of Erk1/2 signaling resulted in increased apoptotic response and blockade of G0-G1 transition in human Schwann cells, a finding that has been previously demonstrated to occur in response to Erk1/2 inhibition by different dominant negative mutants in different cell systems (Brognard and Dennis, 2002, Cell Death Differ. 9:893-904; Roberts et al., 2002, Mol. Cell. Biol. 22:7226-7241).

MEK/Pi3K-Independent Erk1/2 Phosphorylation in Human Schwann Cells is Mediated by PKCα/βII

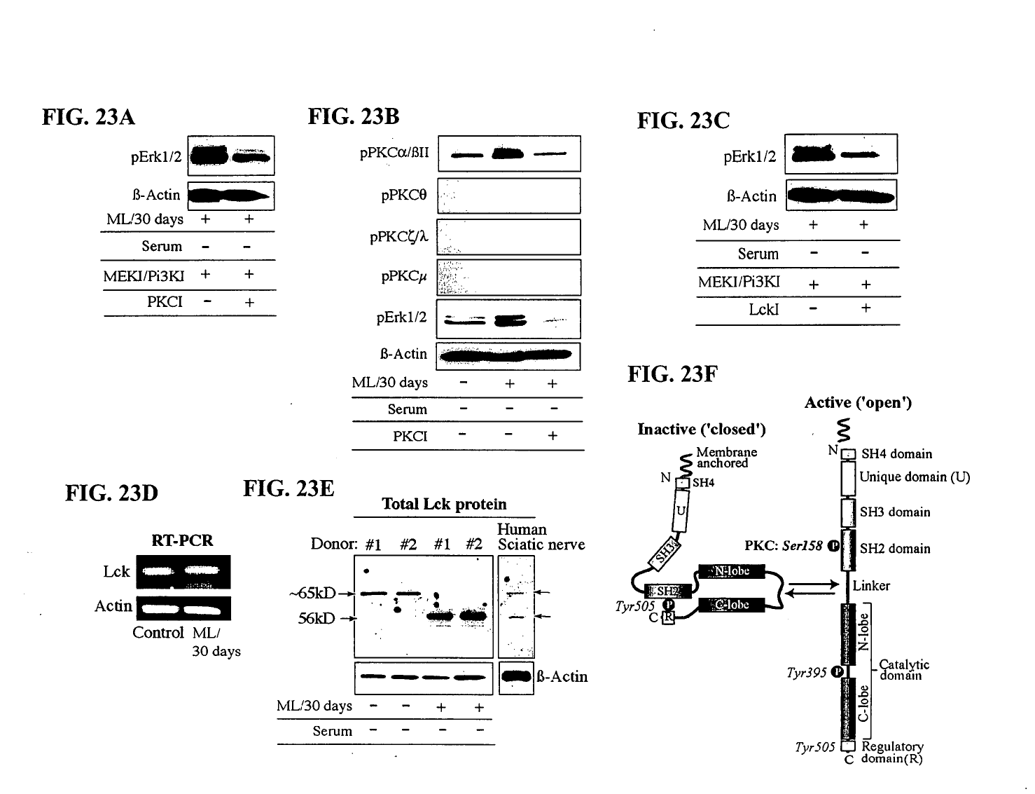

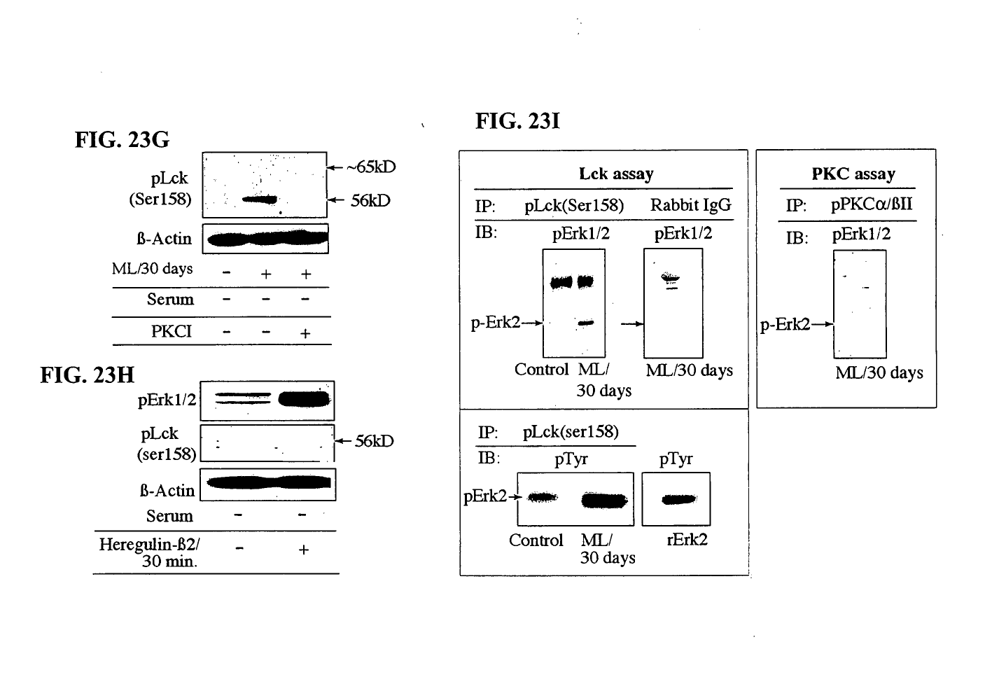

To elucidate the signaling mechanisms by which intracellular M. leprae induce MEK/Pi3K-independent Erk1/2 activation in the absence of extracellular mitogenic signals, 30 day-infected Schwann cells were serum starved for 48 hours with known inhibitors of major signaling pathways together with MEK1/2 and Pi3K inhibitors. It was observed that specific PKC (pan-PKC) inhibitor bisindolylmaleimide-I (20 nM) significantly blocked M. leprae-induced Erk1/2 phosphorylation (FIG. 23A). Treated cells showed no apoptosis or cell death as compared to untreated cells. These data suggest the involvement of PKC in regulating M. leprae-induced Erk1/2 activation in a MEK/Pi3kinase-independent pathway.

The PKC family comprises of about twelve different isoforms that are phospholipid-dependent serine/threonine kinases, and are broadly classified by their activation characteristics. Different isoforms of PKCs can be activated by calcium, various phospholipids, diacylglycerol (DAG) generated from phospholipase C (PLC) or PLD, and fatty acids generated from PLA2 (Mellor and Parker, 1998, Biochem. J. 332:281-92). In mammalian cells, PKC isoforms are divided into three groups: (i) classical PKCs (α, βI, βII, γ), (ii) novel PKCs (δ, ε, η, θ, μ), and (iii) atypical PKCs (ζ, ι), which, depending on the type of isoform, can be activated either by DAG, calcium, or both, or by unknown stimuli (Saijo et al., 2003, Ann. New York Acad. Sci. 987:125-134).

Although PKC is known to play a role in oligodendrocyte proliferation and differentiation (Bhat et al., 1992, J. Neurosci. Res. 32:340-349; Stariha and Kim, 2001, Microsc. Res. Tech. 52:680-688), much less is known about its function in Schwann cells. A panel of specific antibodies directed against the phosphorylated PKC isoforms was used to identify the putative phospho-PKCs in human Schwann cells. It was observed that phospho-PKCα/βII activity was present in lysates of synchronized human Schwann cells, and intracellular M. leprae significantly increased this activity (FIG. 23B). Specificity of M. leprae-induced phospho-PKCα/βII activity was evident as pan-PKC inhibitor markedly blocked the phosphorylation (FIG. 23B).

Interestingly, PKC inhibitor also dramatically down-regulated M. leprae-induced phosphorylation of Erk1/2. Since other phosphorylated PKC isoforms (PKCθ, PKCζ/λ, PKCμ, PKC∂) were undetectable in lysates of human Schwann cells (FIG. 23B), it is most likely that the phosphorylation of PKCα/β II isoform induced by intracellular M. leprae plays an important role in regulating MEK/Pi3K-independent activation of Erk1/2.

The ability of PKCα/βII to serve as a kinase that directly phosphorylate Erk1/2 was examined. In three independent PKCα/βII kinase assay experiments using the total lysates of 30 day-infected Schwann cells as a source of cellular phospho-PKCα/βII and recombinant Erk2 (inactive) as the substrate, phosphorylation of Erk2 (FIG. 23I; right panel) was not observed. Thus, while not wishing to be bound to any particular theory, it is likely that another signaling kinase(s) that serves as a PKCα/βII-activated intermediate(s) is responsible for the MEK/Pi3k-independent Erk1/2 phosphorylation.

Role of p56Lck in MEK/Pi3k-Independent Phosphorylation of Erk1/2 in Human Schwann Cells

Once PKC is active, it phosphorylates a broad range of substrate proteins. Among the kinase substrates of PKC, p56Lck, a member of a non-receptor Src family tyrosine kinases (Soula et al., 1993, J. Biol. Chem. 268:27420-27427), is of special interest, since Lck has been shown to express in both lymphoid tissues and neurons and the recombinant form of murine Lck has been demonstrated to directly phosphorylate Erk1/2 on tyrosine residues in vitro (Ettehadieh et al., 1992, Science 255:853-855). Although the role of Lck in neurons is unknown, its critical role in T-cell antigen receptor signaling, as well as T-cell development, proliferation and survival, has been well characterized (Veillette et al., 1988, Cell 55:301-308; Soula et al., 1993, J. Biol. Chem. 268:27420-27427).

Because Lck is a substrate for PKC, and PKC-inhibitor dramatically down regulated M. leprae-induced Erk1/2 phosphorylation (FIG. 23B), the ability of specific inhibition to Lck to block M. leprae-induced Erk1/2 was examined. When 30 day-infected cells were incubated with Lck inhibitor PP2 (10 μM) in the presence of MEK1/2 and Pi3K inhibitors for 48 hours, a significant reduction of M. leprae-induced phosphorylation of Erk1/2 was observed (FIG. 23C). These data indicates that Lck plays a role in MEK- and Pi3K-independent activation of Erk1/2 in human Schwann cells by M. leprae.

Because the presence of Lck in Schwann cells and peripheral nerves is unknown, the expression of Lck in human primary Schwann cells and human Sciatic nerves (from two different donors) at the gene and protein levels was analyzed. Whereas Lek gene expression in control and infected Schwann cells showed no difference using RT-PCR (FIG. 23D), a major difference in migration pattern of total Lck protein (non-phosphorylated) in SDS-PAGE was observed when immunoblots were reacted with specific antibody to total Lck (FIG. 23E). Total Lck appeared as a slow-migrating band at approximately 65-kDa in the lysates of control human Schwann cells that have been serum starved for 48 hours. On the other hand, in infected cells, grown under similar conditions as the control uninfected cells, Lck showed a prominent band at about 56-kDa (FIG. 23E). Both 56-kDa and ˜65-kDa Lck bands were detected in human Sciatic nerves, suggesting that Lck exists in two forms or configurations in peripheral nerves in situ (FIG. 23E).

The existence of different configurations of Lck upon cell activation has been shown in other systems, but not with respect to M. leprae infection. In T cells, the conformational changes in Lck proteins in inactive and active stages have been studied (Kabouridis, 2003, Biochem. J. 371:907-915; Salter and Kalia, 2004, Nature Rev. Neurosci. 5:317-328). In T-cells, an inactive form of Lck, which is in ‘tail-bite’ configuration and has low enzymatic activity, is found predominantly in lipid rafts and is anchored to the membrane and thus acquires slow migration in SDS-PAGE (Kabouridis, 2003, Biochem. J. 371:907-915). In the active form with much higher enzymatic activity, for instance, after treatment with phorbol esters and subsequent release from lipid rafts, Lck acquires an ‘open’ configuration (Kabouridis, 2003, Biochem. J. 371:907-915). Such configurational changes in the Lck molecule is known to occur by intramolecular interactions as well as by phosphorylation and dephosphorylation (Reynolds et al., 1992, Oncogene 7:1949-1955; Salter and Kalia, 2004, Nature Rev. Neurosci. 5:317-328) (FIG. 23F). The data disclosed herein demonstrate, without wishing to be bound by any particular theory, that the observed migration difference in total Lck in purified Schwann cells demonstrated by SDS-PAGE is likely due to similar configurational changes caused by the activation of the cells by intracellular M. leprae.

Phosphorylation of Lck-Ser-158 in Human Schwann Cells Directly Phosphorylate Erk1/2

Lck is regulated by phosphorylation on multiple residues, including Ser-158 and Ser-194 in the SH2 domain, Tyr-395 in the catalytic domain and Tyr-505 near the C-terminus (Reynolds et al., 1992, Oncogene 7:1949-1955) (FIG. 23F). In T-cell lines, triggering with PKC activator phorbol 12-myrystate 13-acetate (PMA) and anti-CD3 antibody induce distinct phosphorylation sites in the SH2 domain of Lck (Soula et al., 1993, J. Biol. Chem. 268:27420-27427). Whereas T-cell receptor/CD3 triggering results in phosphorylation of Tyr-192 and Ser-194, specific PKC-mediated phosphorylation has been shown at Ser-158 of the active form of Lck molecule (Soula et al., 1993, J. Biol. Chem. 268:27420-27427). Because of the critical involvement of PKC and Lck in M. leprae-induced Erk1/2 regulation (FIGS. 23A-23C), the phosphorylation status of Lck at Ser-158, a site phosphorylated specifically by PKC (Soula et al., 1993, J. Biol. Chem. 268:27420-27427), was analyzed in infected Schwann cells.

Using phospho-specific antibody to Lck/Ser-158, phosphorylation of Ser-158 was detected only in the lysates of M. leprae-infected Schwann cells (FIG. 23G). Unlike the similar expression pattern of total Lck protein detected in infected and control Schwann cells, phospho-Lck/Ser-158 was associated with the 56-kDa protein in infected cells, but not with the approximately 65-kDa protein, which is found only in control cells (FIG. 23E-23G).

Since this phosphorylation at Lck/Ser-158 was completely abolished by PKC inhibitor (10 μM) (FIG. 23G), it is most likely, without wishing to be bound to any particular theory, that activated PKCα/βII is involved in phosphorylation of Lck Ser/158 in infected Schwann cells. Interestingly, unlike intracellular M. leprae, heregulin-β1 (glial growth factor/GGF), a potent extracellular mitogen for Schwann cells that induces Erk1/2 activation via classical MEK-dependent pathway (Zanazzi et al., 2001, J. Cell. Biol. 152:1289-1299) (FIG. 24C), failed to phosphorylate Lck/Ser158 (FIG. 23H).

To assess the role of Lck/Ser-158 in direct phosphorylation of Erk1/2, an in vitro kinase assay was performed using an antibody to phospho-Lck/Ser158. Phospho-Lck/Ser158 was immunoprecipitated from lysates of human Schwann cells and then incubated with recombinant Erk2 (inactive) in the presence of ATP and probed with anti-phospho-Erk1/2 antibody. Immunoprecipitates from M. leprae-infected Schwann cells, but not from controls, directly phosphorylated Erk2 in vitro (FIG. 23I; upper left panel). These results were consistent with Schwann cells isolated from two different human donors. In addition, an in vitro phosphorylation kinase assay was performed using anti-phospho-Tyr antibody.

Since Erk2 can be autophosphorylated on tyrosine residues in the presence of ATP (Ettehadieh et al., 1992, Science 255:853-855), recombinant Erk2 was used as a positive control. Phospho-Lck/Ser158 immunoprecipitated from the lysates of infected Schwann cells showed two-fold increase in phosphorylation of Erk2 on tyrosine residues as compared to autophosphorylated Erk-2 (FIG. 23I; bottom panel). These data together suggest that phosphorylated Lck/Ser158 in human Schwann cells serves as a direct activator of Erk1/2 independent of MEK1/2 (FIG. 24E).

Lck/Ser-158-Mediated Erk1/2 Activation in Human Schwann Cells Induces Nuclear Cyclin D1 and Promotes G1/S-Phase Progression

The data disclosed herein demonstrate that although the specific inhibitors to MEK1/2 are known to block growth factor-induced Erk1/2 activation and subsequent nuclear cyclin D1 and cell cycle progression (Lavoie et al., 1996, J. Biol. Chem. 271:20608-20616), these inhibitors did not affect nuclear cyclin D1 and the net S-phase cell population induced by intracellular M. leprae (FIGS. 24A and 24B). However, addition of Lck inhibitor to the latter combination of inhibitors dramatically reduced the nuclear cyclin D1 and the net S-phase cell population (FIGS. 7A and 7B). Similar reduction was also observed when infected cells were incubated with either PKC inhibitor or Lck inhibitor alone (FIG. 24B). In contrast, Lck inhibitor did not affect the net S-phase Schwann cell population induced by exogenous heregulin-β1 (FIG. 24C), which is inconsistent with the failure to induce phosphorylated Lck/Ser-158 by heregulin-β1 (FIG. 23H). These results show that Lck/Ser-158-dependent Erk1/2 signaling induced by intracellular M. leprae promotes G1/S phase transition of human Schwann cells and this signaling pathway does not appear to operate in heregulin, or extracellular mitogen-induced, cell cycle progression.

A widely accepted concept of mammalian cell cycle research is that the cell cycle entry and G1 phase progression depends solely on signaling from extracellular cues such as mitogens/growth factors (Pardee, 1989, Science 246:603-608; Sherr, 1993, Cell 73:1059-1065; Sherr, 2000, Cancer Res. 60:3689-3695; Roovers and Assoian, 2000, Bioessays 22:818-826). Most of the established views related to the regulation of G1 to S phase transition in normal mammalian cells have been obtained from studying the cell response to extracellular mitogens, particularly on events that occur rapidly after binding of growth factors to their receptors. In this study, taking the advantage of the unique capacity of leprosy bacillus as an intracellular cue to induce human Schwann cell proliferation, it has been demonstrated, for the first time, that signaling triggered from intracellular M. leprae can regulate G1 phase progression and S phase entry in the absence of extracellular mitogenic responses.

Modulating the abundance of cyclins in response to various signaling is one means by which cells control their progression through the G1/S phase of the cell cycle and subsequent proliferation (Sherr, 1995, Trends Biochem. Sci. 20:187-190; Hunter and Pines, 1994, Cell 79:573-582). Regulation of cyclin D1 and G1 phase progression has been extensively studied by examining response of a cell to signaling from extracellular cues, such as the binding of growth factors to receptor tyrosine kinases (RTK) and binding of matrix proteins to integrins (Pardee, 1989, Science 246:603-608; Roovers and Assoian, 2000, Bioessays 22:818-826; Welsh et al., 2001, Nature Cell Biol. 3:950-957). These studies, mostly using fibroblasts, have established that Erk signaling, consisting of Ras, Raf-1, MEK1/2 and Erk1/2, controls the cyclin D1 expression. The data disclosed herein demonstrate that M. leprae residing inside human Schwann cells phosphorylated Erk1/2 independently of extracellular mitogen-induced SOS, Ras, Raf-1, MEK1/2 pathway. The results disclosed herein demonstrate that the novel signaling pathways encompass PKC-alpha-betaII/βII-dependent activation of Lck/Ser-158, which directly phosphorylated Erk1/2. Activation of Erk1/2 via a Lck/Ser-158 pathway induced nuclear accumulation of cyclin-D1, which subsequently lead to G1/S phase progression and continuous Schwann cell proliferation. In contrast, phosphorylation of Lck/Ser-158 did not participate in activation of Erk1/2 by exogenous heregulin-β1/neurgulin-1, a potent mitogen for Schwann cells. Also, pharmacological inhibition of Lck exerts no effect on heregulin-induced S-phase population.

Thus, without wishing to be bound to any particular theory, the signaling mechanisms by which intracellular M. leprae regulate the expression of cyclin D1 and G1/S phase progression in human Schwann cells is different from signaling induced by extracellular mitogens. Since the cell fate can be determined by the nature of signaling the cell received, it is likely that Erk1/2 activation through Lck/Ser-158-dependent and MEK-dependent pathways by intracellular and extracellular cues, respectively, could differentially translate the intracellular and extracellular signals into different cell behavior patterns. The results disclosed herein support such hypothesis, since sustained activation of Erk1/2 via Lck/Ser-158 did not cause transformation despite continuous cell proliferation, whereas sustained activation of Erk1/2 by growth factors via Ras, Raf, MEK-dependent pathway can lead to transformation (Porter and Vaillancourt, 1998, Oncogene 17:1343-1352). It was observed that intracellular M. leprae induce continuous Schwann cell proliferation without transformation, a characteristic feature that is also operative in neural progenitor cells and stem cells (Thomson et al., 1998, Science 282:1145-1147). Current studies are underway to investigate the fate of these infected proliferative Schwann cells and whether sustained activation of such signaling can turn Schwann cells into a highly immature cell stage.

Lck, a Src-family protein kinase that plays an important role in T-cell development, activation and proliferation, and in other hematopoietic cells (Niu and Marcantonio, 2003, Mol. Biol. Cell. 14:349-360), had not been previously shown to be expressed in Schwann cells or oligodendrocytes. It has been demonstrated herein, for the first time, that Lck is expressed in both primary human Schwann cells and in peripheral nerves in situ, and that 56-kDa Lck protein in primary Schwann cells is phosphorylated at Ser-158 in the SH2 domain in response to intracellular M. leprae.

In T-cells, phosphorylation of Lck at Ser-158 is mediated by PKC (Soula et al., 1993, J. Biol. Chem. 268:27420-27427). Although total murine Lck in recombinant form (not cellular Lck) has been shown to phosphorylate Erk1/2 in vitro (Ettehadieh et al., 1992, Science 255:853-855), the site/residues of Lck that is/are responsible for direct phosphorylation of Erk1/2 has not been identified. It has been demonstrated that in human Schwann cells phosphorylation of Lck at Ser-158 is mediated by the activation of PKC isoform PKC-alpha-betaII/βII. Further, MEK-independent Erk1/2 activation is mediated by Lck/Ser-158 via PKC-alpha-betaII/βII pathway, and activation of this signaling is sufficient to induce nuclear accumulation of cyclin D1 and the S-phase entry (FIGS. 24D and 24E). Thus, without wishing to be bound by any particular theory, it is likely that Lck plays an important role in Schwann cell proliferation, which is critical for peripheral nerve development and regeneration (Bunge, 1994, J. Neurol. 242:S19-S21).

Because M. leprae is an obligate intracellular pathogen, propagation of infected Schwann cells is critical for long-term intracellular pathogen survival and to establish productive infection within peripheral nerves, which eventually cause severe neuropathies (Job, 1989, Int. J. Leprosy 57:532-539; Rambukkana et al., 2002, Science 296:927-931). Intracellular bacterial survival can be measured by the viability of M. leprae harvested from long-term infected cells. It has been demonstrated that human Schwann cells infected with viable M. leprae maintained a significant viability after 30 days of infection. Since the propagation of the intracellular niche is critical for the maintenance of M. leprae viability, which is important for the dissemination of infection, disease progression and subsequent nerve damage in vivo, the data presented in this study can be directly linked to the disease pathogenesis thereby providing potential novel therapeutics for this disease.

Finally, the data disclosed herein reveal, for the first time, a surprising but fundamental feature of human cell cycle regulation at the signaling level. That is, the data disclosed herein surprisingly demonstrate the control of G1/S phase transition in response to signals from intracellular cues, which was previously unknown, may reflect a novel regulatory mechanism that controls mammalian cell proliferation. More particularly, it has been demonstrated herein that the activation of Erk1/2 signaling by a MEK-independent and Lck/Ser-158-dependent pathway plays a major role in this novel regulatory mechanism. These studies also reveal that D-type cyclins, in addition to their known role as growth factor sensors (Sherr, 1993, Cell 73:1059-1065; Sherr, 2000, Cancer Res. 60:3689-3695), can also function as sensors for signals from intracellular cues, and thus enable integration of these signals directly with the cell cycle machinery. Because glial cell proliferation is crucial for regeneration of injured nerves in both peripheral and central nervous systems, elucidation of the detailed mechanism of human Schwann cell cycle regulation by M. leprae can provide novel molecular insights into the development of, among other things, new tools for nerve regeneration.

In sum, the data disclosed herein demonstrate a novel mechanism for a M. leprae-induced reprogramming process at the signaling level (shown as a schematic in FIG. 20). Because intracellular M. leprae unusually induce Erk1/2 signaling in human Schwann cells independent of its usual activating partners, i.e., Ras, Raf and MEK (MEK is the only known direct cellular activator of Erk1/2 in response to exogenous growth factors), the data demonstrated that this MEK-independent Erk1/2 activation is mediated by a novel signaling pathway (termed “NSP”) depicted in FIG. 20. The data disclosed herein discloses the identification and characterization of two major components of the NSP, which are responsible for human Schwann cell proliferation in response to intracellular M. leprae.

Infected cells undergo continuous cell proliferation without cellular transformation, a feature characteristic of stem cells. The data disclosed herein connects, for the first time, the NSP deprogramming of Schwann cells by M. leprae to the reprogramming process at the signaling level.

As discussed previously elsewhere herein, cell proliferation is a result of the completion of cell cycle progression from G1, S, G2, to M phases. G1/S phase transition is critical for the initiation and continuation of cell cycle progression and the transition is normally regulated by signaling from extracellular mitogens, such as growth factors. Such receptor-mediated growth factor-induced activation of signaling is integrated to cell cycle machinery via cyclin D1, which regulates cell cycle progression to completion. One of the well-characterized signaling pathways that regulate cyclin D1 expression is Erk1/2 (p42/44 MAP kinase). Erk1/2 is usually phosphorylated by sequential activation of Ras, Raf and MEK pathway upon stimulation by extracellular mitogens (see FIG. 24D). Surprisingly, in human Schwann cells infected with M. leprae for 30 days, cyclin D1 is highly up-regulated and its expression, as well as subsequent G1/S phase transition by the cells, are regulated by Erk1/2 independent of Ras, Raf and MEK pathway. Interestingly, in mouse embryonic stem (ES) cells, which are highly proliferative, cyclin D1 expression and G1 phase cell cycle progression are also regulated independent of the Ras, Raf and MEK pathway. Thus, the data disclosed herein suggest, for the first time, that the novel MEK-independent signaling pathway exemplified by M. leprae-infected cells, is involved in regulation of ES cell cycle progression.

Erk1/2 participates in a wide range of cellular processes from cell proliferation, differentiation, development, to memory functions, and the route or degree of activation may be important for the outcome of a given cellular process. The signaling pathway that phosphorylates Erk1/2 by M. leprae from inside human Schwann cells was identified as MEK-independent and PKC alpha/beta II- and p56Lck (lymphoid cell kinase)-dependent Erk1/2 activation (see schematic depicted in FIG. 24E). p56Lck is a key player in this novel signaling pathway, since the Ser-158 residue of Lck directly phosphorylates Erk1/2 (the usual known cellular substrate for Erk1/2 is MEK).

Surprisingly, Lck/Ser 158-dependent Erk1/2 activation is not operated in response to extracellular mitogens, and thus does not seem to function in adult differentiated Schwann cells. On the other hand, Lck is critical for thymus and T cell development, and therefore, likely to be involved in embryonic development. Strikingly, recent studies of human ES cell “stemness” gene analysis reveal that Lck is one of the ES cell enriched genes in undifferentiated human ES cells (Sato et al., 2003, Dev. Biol. 260:404). These studies together with the data disclosed herein suggest that Lck/Ser-158 functions in the embryonic stage and that intracellular M. leprae (and/or M. leprae components) has the capacity to activate such signaling transducers otherwise involved in embryonic stage but not in differentiated cells. Indeed, during development, many signaling pathways appear, disappear, reappear and disappear, and certain signaling that is operative in the embryonic stage may not be functional in adult differentiated cells. In adult human Schwann cells, expression of Lck (particularly phosphorylated Lck/Ser-158) has not been identified before the present study.

Therefore, without wishing to be bound by any particular theory, M. leprae may activate a signaling pathway in differentiated cells that is normally operative in embryonic stage but not in the differentiated cells. Thus, the differentiated cells can be reprogrammed back to embryonic-like or immature stage using the disclosure provided herein. Likewise, the induction of Lck-mediated Erk1/2 activation by intracellular M. leprae, which leads to continuous cell proliferation, can be the signaling that initiates cellular reprogramming process. Therefore, an activator that directly targets Lck activation (sustained activation) may initiate the reprogramming process. In this regard, it is important to note that an earlier study has shown that the treatment of adult oligodendrocytes, the glial cell of the CNS, with PKC activators transiently reversed the mature oligodendrocyte cells into an immature stage (Avossa and Pfeiffer, 1993, J. Neurosci. Res. 34:113). However, Avossa and Pfeiffer demonstrated only brief reversible de-differentiation of oligodendrocytes to oligodendrocyte-precursor stage and not to neural stem cell stage. Oligodendrocyte-precursor stage should be able to be further reprogrammed or should go backwards/reverse to become neural stem cell stage, but this was not achieved previously. The data disclosed herein now demonstrate that since PKC is responsible for M. leprae-induced phosphorylation of Lck/Ser158 and subsequent activation of Erk1/2 (see schematic in FIG. 24E), it is possible that small molecule activators of LCK or PKC, or combination of both, can mimic reprogramming of adult cells to stem cell-like cells.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are to be construed to include all such embodiments and equivalent variations.

What is claimed:

1. A method of producing a reprogrammed embryonic stem-cell like cell (ES-like cell), said method comprising contacting a differentiated adult cell with a Mycobacterium leprae bacterium, or a component thereof, thereby producing said reprogrammed ES-like cell.

2. The method of claim 1, wherein said bacterium is viable or non-viable.

3. The method of claim 1, wherein said adult differentiated cell is a eukaryotic cell.

4. The method of claim 3, wherein said eukaryotic cell is a mammalian cell.

5. The method of claim 4, wherein said mammalian cell is selected from the group consisting of a Schwann cell, a keratinocyte, a beta-islet cell, a hepatocyte, and a heart muscle cell.

6. The method of claim 5, wherein said cell is a Schwann cell.

7. The method of claim 1, wherein said differentiated cell is incubated with said bacterium, or said component thereof, for at least 15 to about 30 days.

8. The method of claim 1, wherein said component is at least one component selected from the group consisting of a PGL-1, a whole cell wall fraction, a cell wall protein, a cell wall lipid, a cell wall carbohydrate, a protein released from a viable bacterium, and a protein secreted from a viable bacterium.

9. A cell produced by the method of claim 1.

10. The method of claim 1, said method further comprising converting said reprogrammed cell to a stem cell-like cell, wherein said conversion comprises contacting said reprogrammed cell with a progenitor medium, thereby converting said reprogrammed cell to a stem cell-like cell.

11. A cell produced by the method of claim 10.

12. An isolated ES-like cell, wherein said cell is produced by contacting an adult differentiated cell with a Mycobacterium leprae bacterium, or a component thereof.

13. The isolated ES-like cell of claim 12, wherein said bacterium is selected from the group consisting of a viable bacterium and an irradiated bacterium.

14. A method of producing a re-differentiated cell, said method comprising
(a) contacting a differentiated adult cell with a Mycobacterium leprae bacterium, or a component thereof, thereby producing a reprogrammed ES-like cell;
(b) incubating said reprogrammed ES-like cell in a progenitor cell medium, thereby producing a stem cell-like cell and;
(c) re-differentiating said stem cell-like cell into a differentiated cell of the same cell type as said differentiated adult cell, thereby producing a re-differentiated cell.

15. The method of claim 14, wherein said differentiated adult cell is an adult human Schwann cell, and wherein said re-differentiated cell is a neural-like cell.

16. The method of claim 15, further wherein said neural-like cell is selected from the group consisting of a neuron-like cell and an oligodendrocyte-like cell.

17. A re-differentiated cell produced using the method of claim 14.

18. The method of claim 14, wherein said stem cell-like cell of step (b) is grown to produce at least two stem cell-like cells prior to the re-differentiation of (c).

19. A method of producing a trans-differentiated cell, said method comprising
(a) contacting a differentiated adult cell with a Mycobacterium leprae bacterium, or a component thereof, thereby producing a reprogrammed ES-like cell;
(b) incubating said reprogrammed ES-like cell in a progenitor cell medium, thereby producing a stem cell-like cell and;
(c) trans-differentiating said stem cell-like cell into a differentiated cell of a different cell lineage than said differentiated adult cell,
thereby producing a trans-differentiated cell.

20. A trans-differentiated cell produced using the method of claim 19.

21. The method of claim 19, wherein said stem cell-like cell of step (b) is grown to produce at least two stem cell-like cells prior to the trans-differentiation of (c).

22. A method of producing a neurosphere, said method comprising contacting a differentiated adult Schwann cell with a Mycobacterium leprae bacterium, or a component thereof, thereby reprogramming said Schwann cell to produce an ES-like cell, and incubating said reprogrammed ES-like cell in a progenitor medium, thereby producing a neurosphere.

23. An isolated neurosphere produced by the method of claim 22.

24. A method of producing a neuron-like cell, said method comprising contacting a differentiated adult Schwann cell with a Mycobacterium leprae bacterium, or a component thereof, thereby reprogramming said Schwann cell into an ES-like cell, incubating said reprogrammed ES-like cell in a progenitor medium to produce a neurosphere, and incubating said neurosphere in a neuronal differentiation medium, thereby producing a neuron-like cell.

25. A neuron-like cell produced using the method of claim 24.

26. A method of producing an oligodendrocyte-like cell, said method comprising contacting a differentiated adult Schwann cell with a Mycobacterium leprae bacterium, or a component thereof, thereby reprogramming said Schwann cell into an ES-like cell, incubating said reprogrammed ES-like cell in a progenitor medium to produce a neurosphere, and incubating said neurosphere in an oligodendrocyte differentiation medium, thereby producing an oligodendrocyte-like cell.

27. An oligodendrocyte-like cell produced using the method of claim 26.

28. A method of affecting the passage of a cell through the cell cycle, said method comprising contacting a cell with a Mycobacterium leprae bacterium, or a component thereof, thereby affecting the passage of said cell through the cell cycle.

29. A method of increasing the level of cyclin D1 expression in a cell, said method comprising contacting a cell with a Mycobacterium leprae bacterium, or component thereof, thereby increasing the level of cyclin D1 expression in said cell.

30. A method of inducing MEK/Pi3K-independent phosphorylation of Erk1/2 in a cell, said method comprising contacting a cell with a Mycobacterium leprae bacterium, or component thereof, thereby inducing MEK/Pi3K-independent phosphorylation of Erk1/2 in said cell.

31. The method of claim 30, said method comprising phosphorylating the serine 158 amino acid residue of Lck.

32. A method of inducing MEK/Pi3K-independent phosphorylation of GSK3β in a cell, said method comprising contacting a cell with a Mycobacterium leprae bacterium, or component thereof, thereby inducing MEK/Pi3K-independent phosphorylation of GSK3β in said cell.

33. The method of claim 32, said method further comprising phosphorylating the serine 158 amino acid residue of Lck.

34. A method of identifying a component a Mycobacterium leprae bacterium that reprograms a differentiated cell, said method comprising contacting a differentiated adult cell with a component of a Mycobacterium leprae bacterium, and comparing the level of a marker of an undifferentiated state in said cell contacted with said component with the level of said marker in an otherwise identical cell not contacted with said component, wherein a higher level said marker in said cell contacted with said component compared with said level of said marker in said cell not contacted with said component is an indication that said component reprogrammed said cell, thereby identifying a component a Mycobacterium leprae bacterium that reprograms a differentiated cell.

35. The method of claim 34, wherein said marker is selected from the group consisting of the level of cyclin D1 expression in a cell, the level of p21 expression in a cell, the level of phosphorylation of Lck at serine residue number 158, the level of proliferation without transformation, the level of MEK/Pi3K-independent Erk1/2 phosphorylation, the level of MEK/Pi3K-independent GSK3β phosphorylation, expression of an embryonic-stage marker protein, and expression of an embryonic-stage gene.

36. A component identified by the method of claim 34.

37. A method of identifying a component of a Mycobacterium leprae bacterium that affects progression of a cell through the cell cycle, said method comprising contacting a cell with a component of a Mycobacterium leprae bacterium, and comparing the progression of said cell through the cell cycle with the progression through the cell cycle by an otherwise identical cell not contacted with said component, wherein a faster progression through said cell cycle by said cell contacted with said component compared with said progression through said cell cycle by said cell not contacted with said component is an indication that said component affects progression of a cell through the cell cycle, thereby identifying a component of a Mycobacterium leprae bacterium that affects progression of a cell through the cell cycle.

38. A component identified by the method of claim 37.

39. A method of identifying a component of a Mycobacterium leprae bacterium that increases cell proliferation, said method comprising contacting a cell with a component of a Mycobacterium leprae bacterium, and comparing the level of proliferation of said cell with the level of proliferation of an otherwise identical cell not contacted with said component, wherein a greater level of proliferation of said cell contacted with said component compared with said level of proliferation of said otherwise identical cell not contacted with said component is an indication that said component increases cell proliferation, thereby identifying a component of a Mycobacterium leprae bacterium that increases cell proliferation.

40. A component identified by the method of claim 39.

41. A method of treating a neurological disease in an animal in need thereof, said method comprising administering to said animal an effective amount of an ES-like cell produced by the method of claim 1, thereby treating a neurological disease in said animal.

42. The method of claim 41, wherein said neurological disease is selected from the group consisting of Alzheimer's disease, Parkinson's disease, central nervous system injury, and multiple sclerosis.

43. The method of claim 42, wherein the central nervous system injury is selected from the group consisting of spinal cord injury, stroke, ischemia, and brain injury.

44. A method of treating a neurological disease in an animal in need thereof, said method comprising administering to said animal an effective amount of a neuron-like cell produced by the method of claim 24, thereby treating a neurological disease in said animal.

45. A method of treating a neurological disease in an animal in need thereof, said method comprising administering to said animal an effective amount of a oligodendrocyte-like cell produced by the method of claim 26, thereby treating a neurological disease in said animal.

46. A kit for reprogramming an adult differentiated cell, said kit comprising an irradiated Mycobacterium leprae bacterium, or a component thereof, and said kit further comprising an applicator and an instructional material for use thereof.

47. The kit of claim 46, said kit further comprising a progenitor medium.

48. The kit of claim 46, said kit further comprising a differentiation medium.

49. The kit of claim 46, wherein said component is a cell wall obtained from a Mycobacterium leprae bacterium.
